SEQUENCE LISTING (164 sequences)

SEQ ID NO: 1 - protein, 15 aa, Artificial Sequence
  synthetic
SHLVEALYLVCGERG

SEQ ID NO: 2 - protein, 15 aa, Artificial Sequence
  synthetic
SHLVEALYLVCGEEG

SEQ ID NO: 3 - protein, 16 aa, Artificial Sequence
  synthetic
SHLVEELYVLVCGEEG

SEQ ID NO: 4 - protein, 15 aa, Artificial Sequence
  synthetic
GQVELGGGNAVEVLK

SEQ ID NO: 5 - protein, 15 aa, Artificial Sequence
  synthetic
LQVELGGGPGAGSLQ

SEQ ID NO: 6 - protein, 18 aa, Artificial Sequence
  synthetic
GSLQPLALEGSLQKRGIV

SEQ ID NO: 7 - protein, 8 aa, Artificial Sequence
  synthetic
WGPDPAAA

SEQ ID NO: 8 - protein, 15 aa, Artificial Sequence
  synthetic
SHLVEALVLVCGERG

SEQ ID NO: 9 - protein, 10 aa, Artificial Sequence
  synthetic
VMNILLQYVV

SEQ ID NO: 10 - protein, 18 aa, Artificial Sequence
  synthetic
TAGTTVYGAFDPLLAVAD

SEQ ID NO: 11 - protein, 22 aa, Artificial Sequence
  synthetic
VNFFRMVISNPAATHQDIDFLI

SEQ ID NO: 12 - protein, 9 aa, Artificial Sequence
  synthetic
VIVMLTPLV

SEQ ID NO: 13 - protein, 20 aa, Artificial Sequence
  synthetic
SFYLKNVQTQETRTLTQFHF

SEQ ID NO: 14 - protein, 13 aa, Artificial Sequence
  synthetic
QHLQKDYRAYYTF

SEQ ID NO: 15 - protein, 13 aa, Artificial Sequence
  synthetic
YTFLNFMSNVGDP

SEQ ID NO: 16 - protein, 13 aa, Artificial Sequence
  synthetic
RVLNIDLLWSVPI

SEQ ID NO: 17 - protein, 13 aa, Artificial Sequence
  synthetic
DWIHIDTTPFAGL

SEQ ID NO: 18 - protein, 14 aa, Artificial Sequence
  synthetic
WSKMDQLAKELTAE

SEQ ID NO: 19 - protein, 9 aa, Artificial Sequence
  synthetic
VAANIVLTV

SEQ ID NO: 20 - protein, 20 aa, Artificial Sequence
  synthetic
MEFLERTYLVNDKAAKMHAF

SEQ ID NO: 21 - protein, 20 aa, Artificial Sequence
  synthetic
YLVNDKAAKMHAFTLESVEL

SEQ ID NO: 22 - protein, 20 aa, Artificial Sequence
  synthetic
SLWLSSKPPSKRLTFGWHRA

SEQ ID NO: 23 - protein, 20 aa, Artificial Sequence
  synthetic
PPSKRLTFGWHRAEILGALL

SEQ ID NO: 24 - protein, 20 aa, Artificial Sequence
  synthetic
FIFSILVLASTITILKDFSI

SEQ ID NO: 25 - protein, 20 aa, Artificial Sequence
  synthetic
LKDFSILLMEGVPKSLNYSG

SEQ ID NO: 26 - protein, 20 aa, Artificial Sequence
  synthetic
SLNYSGVKELILAVDGVLSV

SEQ ID NO: 27 - protein, 20 aa, Artificial Sequence
  synthetic
FGWHRAEILGALLSILCIWV

SEQ ID NO: 28 - protein, 20 aa, Artificial Sequence
  synthetic
TMNQVILSAHVATAASRDSQ

SEQ ID NO: 29 - protein, 20 aa, Artificial Sequence
  synthetic
KFGADARALMLQGVDLLADA

SEQ ID NO: 30 - protein, 20 aa, Artificial Sequence
  synthetic
NPVEIRRGVMLAVDAVIAEL

SEQ ID NO: 31 - protein, 21 aa, Artificial Sequence
  synthetic
QSIVPALEIANAHRKPLVIIA

SEQ ID NO: 32 - protein, 20 aa, Artificial Sequence
  synthetic
LVLNRLKVGLQVVAVKAPGF

SEQ ID NO: 33 - protein, 20 aa, Artificial Sequence
  synthetic
IVLGGGCALLRCIPALDSLT

SEQ ID NO: 34 - protein, 20 aa, Artificial Sequence
  synthetic
VNMVEKGIIDPTKVVRTALL

SEQ ID NO: 35 - protein, 16 aa, Artificial Sequence
  synthetic
SPSLWEIEFAKQLASV

SEQ ID NO: 36 - protein, 13 aa, Artificial Sequence
  synthetic
QDFTNRINKLKNS

SEQ ID NO: 37 - protein, 13 aa, Artificial Sequence
  synthetic
  MOD_RES (6)..(6): x  is citruline
QDFTNXINKLKNS

SEQ ID NO: 38 - protein, 20 aa, Artificial Sequence
  synthetic
VVLLVATEGRVRVNSAYQDK

SEQ ID NO: 39 - protein, 20 aa, Artificial Sequence
  synthetic
  MOD_RES (10)..(10): x is citruline
VVLLVATEGXVRVNSAYQDK

SEQ ID NO: 40 - protein, 13 aa, Artificial Sequence
  synthetic
SAVRARSSVPGVR

SEQ ID NO: 41 - protein, 15 aa, Artificial Sequence
  synthetic
SAVRACITSSVPGVR

SEQ ID NO: 42 - protein, 13 aa, Artificial Sequence
  synthetic
QYMRADQAAGGLR

SEQ ID NO: 43 - protein, 15 aa, Artificial Sequence
  synthetic
QYMCITADQAAGGLR

SEQ ID NO: 44 - protein, 13 aa, Artificial Sequence
  synthetic
AGFKGEQGPKGEP

SEQ ID NO: 45 - protein, 15 aa, Artificial Sequence
  synthetic
AGFKGGGEQGPKGEP

SEQ ID NO: 46 - protein, 21 aa, Artificial Sequence
  synthetic
MEVGWYRPPFSRVVHLYRNGK

SEQ ID NO: 47 - protein, 13 aa, Artificial Sequence
  synthetic
HCLGKWLGHPDKF

SEQ ID NO: 48 - protein, 15 aa, Artificial Sequence
  synthetic
ENPVVHFFKNIVTPR

SEQ ID NO: 49 - protein, 20 aa, Artificial Sequence
  synthetic
KYLATASTMDHARHGFLPRH

SEQ ID NO: 50 - protein, 19 aa, Artificial Sequence
  synthetic
LSRFSWGAEGQRPGFGYGG

SEQ ID NO: 51 - protein, 25 aa, Artificial Sequence
  synthetic
AQGTLSKIFKLGGRDSRSGSPMARR

SEQ ID NO: 52 - protein, 9 aa, Artificial Sequence
  synthetic
YLSGADLNL

SEQ ID NO: 53 - protein, 13 aa, Artificial Sequence
  synthetic
LWWVNNQSLPVSP

SEQ ID NO: 54 - protein, 17 aa, Artificial Sequence
  synthetic
QLQPFPQPELPYPQPQP

SEQ ID NO: 55 - protein, 10 aa, Artificial Sequence
  synthetic
QLIPCMDVVL

SEQ ID NO: 56 - protein, 15 aa, Artificial Sequence
  synthetic
YLQLQPFPQPQLPYP

SEQ ID NO: 57 - protein, 15 aa, Artificial Sequence
  synthetic
PFPQPQLPYPQPQLP

SEQ ID NO: 58 - protein, 15 aa, Artificial Sequence
  synthetic
QLPYPQPQLPYPQPQ

SEQ ID NO: 59 - protein, 15 aa, Artificial Sequence
  synthetic
QPQLPYPQPQLPYPQ

SEQ ID NO: 60 - protein, 15 aa, Artificial Sequence
  synthetic
YPQPQLPYPQPQPFR

SEQ ID NO: 61 - protein, 15 aa, Artificial Sequence
  synthetic
YPSGQGSFQPSQQNP

SEQ ID NO: 62 - protein, 15 aa, Artificial Sequence
  synthetic
GSFQPSQQNPQAQGS

SEQ ID NO: 63 - protein, 15 aa, Artificial Sequence
  synthetic
QAQGSVQPQQLPQFE

SEQ ID NO: 64 - protein, 12 aa, Artificial Sequence
  synthetic
QLQPFPQPELPY

SEQ ID NO: 65 - protein, 11 aa, Artificial Sequence
  synthetic
PQPELPYPQPE

SEQ ID NO: 66 - protein, 12 aa, Artificial Sequence
  synthetic
QQPFPQPEQPFP

SEQ ID NO: 67 - protein, 12 aa, Artificial Sequence
  synthetic
FPQPEQPFPWQP

SEQ ID NO: 68 - protein, 12 aa, Artificial Sequence
  synthetic
QGIIQPEQPAQL

SEQ ID NO: 69 - protein, 14 aa, Artificial Sequence
  synthetic
SGEGSFQPSQENPQ

SEQ ID NO: 70 - protein, 12 aa, Artificial Sequence
  synthetic
FPEQPEQPYPEQ

SEQ ID NO: 71 - protein, 15 aa, Artificial Sequence
  synthetic
KVSFFCKNKEKKCSY

SEQ ID NO: 72 - protein, 15 aa, Artificial Sequence
  synthetic
VPVKKATVVYQGERV

SEQ ID NO: 73 - protein, 22 aa, Artificial Sequence
  synthetic
SCKLVPVKKATVVYQGERVKIQ

SEQ ID NO: 74 - protein, 19 aa, Artificial Sequence
  synthetic
MISPVLILFSSFLCHVIAG

SEQ ID NO: 75 - protein, 17 aa, Artificial Sequence
  synthetic
QATQKITYRISGVGIDQ

SEQ ID NO: 76 - protein, 17 aa, Artificial Sequence
  synthetic
PFGIFVVDKNTGDINIT

SEQ ID NO: 77 - protein, 17 aa, Artificial Sequence
  synthetic
HLNSKIAFKIVSQEPAG

SEQ ID NO: 78 - protein, 17 aa, Artificial Sequence
  synthetic
GTPMFLLSRNTGEVRTL

SEQ ID NO: 79 - protein, 17 aa, Artificial Sequence
  synthetic
QCECNIKVKDVNDNFPM

SEQ ID NO: 80 - protein, 17 aa, Artificial Sequence
  synthetic
SVKLSIAVKNKAEFHQS

SEQ ID NO: 81 - protein, 17 aa, Artificial Sequence
  synthetic
NVREGIAFRPASKTFTV

SEQ ID NO: 82 - protein, 17 aa, Artificial Sequence
  synthetic
IYVNVEPTFQRTLHKTK

SEQ ID NO: 83 - protein, 20 aa, Artificial Sequence
  synthetic
GEIRTMNNFLDREIYVNVEP

SEQ ID NO: 84 - protein, 19 aa, Artificial Sequence
  synthetic
MNNFLDREIYNVEPTFQRT

SEQ ID NO: 85 - protein, 20 aa, Artificial Sequence
  synthetic
DREIYVNVEPTFQRTLHKTK

SEQ ID NO: 86 - protein, 20 aa, Artificial Sequence
  synthetic
TLTLLPLLANNRERRGIALD

SEQ ID NO: 87 - protein, 19 aa, Artificial Sequence
  synthetic
NRERRGIALDGKIKHEDTL

SEQ ID NO: 88 - protein, 20 aa, Artificial Sequence
  synthetic
LLANNRERRGIALDGKIKHE

SEQ ID NO: 89 - protein, 20 aa, Artificial Sequence
  synthetic
ASSTIIKEGIDRTVLGILVS

SEQ ID NO: 90 - protein, 19 aa, Artificial Sequence
  synthetic
YQIKVKLTVSGFGELTSSE

SEQ ID NO: 91 - protein, 19 aa, Artificial Sequence
  synthetic
MAASGKTSKSEPNHVIFKK

SEQ ID NO: 92 - protein, 20 aa, Artificial Sequence
  synthetic
QVQPVDGVVLVDPDLVKGKK

SEQ ID NO: 93 - protein, 20 aa, Artificial Sequence
  synthetic
VYVTLTCAFRYGQEDVDVIG

SEQ ID NO: 94 - protein, 20 aa, Artificial Sequence
  synthetic
LTFRRDLYFSRVQVYPPVGA

SEQ ID NO: 95 - protein, 20 aa, Artificial Sequence
  synthetic
PFLLTFPDYLPCSVMLQPAP

SEQ ID NO: 96 - protein, 20 aa, Artificial Sequence
  synthetic
QDSGKSCGVDFEVKAFATDS

SEQ ID NO: 97 - protein, 20 aa, Artificial Sequence
  synthetic
TDAEEDKIPKKSSVRYLIRS

SEQ ID NO: 98 - protein, 20 aa, Artificial Sequence
  synthetic
FMSDKPLHLAVSLNREIYFH

SEQ ID NO: 99 - protein, 20 aa, Artificial Sequence
  synthetic
GEPIPVTVTVTNNTEKTVKK

SEQ ID NO: 100 - protein, 20 aa, Artificial Sequence
  synthetic
IKACVEQVANVVLYSSDYYV

SEQ ID NO: 101 - protein, 20 aa, Artificial Sequence
  synthetic
GKIKHEDTNLASSTIIKEGI

SEQ ID NO: 102 - protein, 13 aa, Artificial Sequence
  synthetic
GELTSSEVATEVP

SEQ ID NO: 103 - protein, 11 aa, Artificial Sequence
  synthetic
LTSSEVATEVP

SEQ ID NO: 104 - protein, 38 aa, Artificial Sequence
  synthetic
FKDYSSVVRPVEDHRQVVEVTVGLQLIQLINVDEVNQI

SEQ ID NO: 105 - protein, 17 aa, Artificial Sequence
  synthetic
LGTWTYDGSVVAINPES

SEQ ID NO: 106 - protein, 41 aa, Artificial Sequence
  synthetic
VKKIHIPSEKIWRPDLVLYNNADGDFAIVKFTKVLLQYTGH

SEQ ID NO: 107 - protein, 16 aa, Artificial Sequence
  synthetic
IHIPSEKIWRPDLVLY

SEQ ID NO: 108 - protein, 16 aa, Artificial Sequence
  synthetic
LGTWTYDGVVAINPES

SEQ ID NO: 109 - protein, 18 aa, Artificial Sequence
  synthetic
DPTYLDITYHFVMQRLPL

SEQ ID NO: 110 - protein, 18 aa, Artificial Sequence
  synthetic
DTPYLDITYHFVMQRLPL

SEQ ID NO: 111 - protein, 13 aa, Artificial Sequence
  synthetic
VIVELIPSTSSAV

SEQ ID NO: 112 - protein, 23 aa, Artificial Sequence
  synthetic
KSYCEIIVTHFPFDEQNCSMKLG

SEQ ID NO: 113 - protein, 20 aa, Artificial Sequence
  synthetic
PRWNGEKLYQEARKIVGAMV

SEQ ID NO: 114 - protein, 32 aa, Artificial Sequence
  synthetic
DLGWGVVGTHAAPAHGQALGAVGHWLVLLWQL

SEQ ID NO: 115 - DNA, 23 bp, Artificial Sequence
  synthetic
ctactgaagtatacgtaaagtgg

SEQ ID NO: 116 - DNA, 23 bp, Artificial Sequence
  synthetic
cttgtttagagctccatcaaagg

SEQ ID NO: 117 - DNA, 23 bp, Artificial Sequence
  synthetic
atcgagctaaaatatcgtgttgg

SEQ ID NO: 118 - DNA, 23 bp, Artificial Sequence
  synthetic
ttcctttccaggatcagctcagc

SEQ ID NO: 119 - DNA, 24 bp, Artificial Sequence
  synthetic
ttgattcaaaggagtacctatccc

SEQ ID NO: 120 - DNA, 645 bp, Artificial Sequence
  synthetic
atggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctg

SEQ ID NO: 121 - protein, 215 aa, Artificial Sequence
  synthetic
MDDQRDLISNHEQLPILGNRPREPERCSRGALYTGVSVLVALLLAGQATTAYFLYQQQGRLDKLTITSQNLQLESLRMKLPKSAKPVSQMRMATPLLMRPMSMDNMLLGPVKNVTKYGNMTQDHVMHLLTRSGPLEYPQLKGTFPENLKHLKNSMDGVNWKIFESWMKQWLLFEMSKNSLEEKKPTEAPPKEPLDMEDLSSGLGVTRQELGQVTL

SEQ ID NO: 122 - DNA, 126 bp, Artificial Sequence
  synthetic
tgtggcatctcctcagcagagagcctgaagatatctcaagctgtccatgcagcacatgcagaaatcaatgaagcaggcagagaggtggtagggtcagcagaggctggagtggatgctgcaagctga

SEQ ID NO: 123 - protein, 41 aa, Artificial Sequence
  synthetic
CGISSAESLKISQAVHAAHAEINEAGREVVGSAEAGVDAAS

SEQ ID NO: 124 - DNA, 345 bp, Artificial Sequence
  synthetic
tgcatgttggtgctgttgcctgatgaagtctcaggccttgagcagcttgagagtataatcaactttgaaaaactgactgaatggaccagttctaacgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgccagctga

SEQ ID NO: 125 - protein, 114 aa, Artificial Sequence
  synthetic
CMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAAS

SEQ ID NO: 126 - DNA, 81 bp, Artificial Sequence
  synthetic
cagcacctttgtggttcccacctggtggaggctctctacctggtgtgtggggagcgtggcttcttctacacacccatgtaa

SEQ ID NO: 127 - protein, 26 aa, Artificial Sequence
  synthetic
QHLCGSHLVEALYLVCGERGFFYTPM

SEQ ID NO: 128 - DNA, 81 bp, Artificial Sequence; identical to SEQ ID NO: 126
  synthetic

SEQ ID NO: 129 - protein, 26 aa, Artificial Sequence
  synthetic
QHLCGSHLVEALYLVCGEEGFFYTPM

SEQ ID NO: 130 - DNA, 256 bp, Artificial Sequence
  synthetic
cacacaaatgtctgcttctggtttgtacctcctagtttgcgcactctggaagacaatgaagagagaatgagccgcctctcaaaggtggcgccagtgattaaagccagaatgatggagtatgggaccacaatggtcagctaccaacccttaggggacaaggtcaacttcttccgcatggtcatctcaaaccctgcagcaactcaccaagacattgacttccttattgaagaaatcgaacgcctcggacaagatttgt

SEQ ID NO: 131 - protein, 85 aa, Artificial Sequence
  synthetic
HTNVCFWFVPPSLRTLEDNEERMSRLSKVAPVIKARMMEYGTTMVSYQPLGDKVNFFRMVISNPAATHQDIDFLIEEIERLGQDL

SEQ ID NO: 132 - DNA, 75 bp, Artificial Sequence
  synthetic
actaacatgttcacctatgagatcgcccctgtatttgtgctgctagaatatgttacactaaagaaaatgagataa

SEQ ID NO: 133 - protein, 24 aa, Artificial Sequence
  synthetic
TNMFTYEIAPVFVLLEYVTLKKMR

SEQ ID NO: 134 - DNA, 87 bp, Artificial Sequence
  synthetic
gaggcctttgaacacactccaggagtccacatggccagcttgagtgtgtacctgaagaccaacgtcttcctcttcctgtttgcctaa

SEQ ID NO: 135 - protein, 28 aa, Artificial Sequence
  synthetic
EAFEHTPGVHMASLSVYLKTNVFLFLFA

SEQ ID NO: 136 - DNA, 123 bp, Artificial Sequence
  synthetic
gaggtggaggaccctcaggtggcccagctggagctgggcggcggccctggcgccggcgacctgcagaccctggccctgtggagcagaatggaccagctggccaaggagctgaccgccgagtga

SEQ ID NO: 137 - protein, 40 aa, Artificial Sequence
  synthetic
EVEDPQVAQLELGGGPGAGDLQTLALWSRMDQLAKELTAE

SEQ ID NO: 138 - DNA, 435 bp, Artificial Sequence
  synthetic
atggatgaccagcgcgaccttatctccaacaatgagcaactgcccatgctgggccggcgccctggggccccggagagcaagtgcagccgcggagccctgtacacaggcttttccatcctggtgactctgctcctcgctggccaggccaccaccgcctacttcctgtaccagcagcagggccggctggacaaactgacagtcacctcccagaacctgcagctggagaacctgcgcatgaagcttcccaagcctcccaagcctgtgagcaagatgcgcatggccaccccgctgctgatgcaggcgctgcccatgggagccctgccccaggggcccatgcagaatgccaccaagtatggcaacatgacagaggaccatgtgatgcacctgctccagagtcactggaactggaggacccgtcttctgggctgggtgtga

SEQ ID NO: 139 - protein, 144 aa, Artificial Sequence
  synthetic
MDDQRDLISNNEQLPMLGRRPGAPESKCSRGALYTGFSILVTLLLAGQATTAYFLYQQQGRLDKLTVTSQNLQLENLRMKLPKPPKPVSKMRMATPLLMQALPMGALPQGPMQNATKYGNMTEDHVMHLLQSHWNWRTRLLGWV

SEQ ID NO: 140 - DNA, 78 bp, Artificial Sequence
  synthetic
caacacctgtgcggctcacacctggtggaagctctctacctagtgtgcggggaacgaggcttcttctacacacccaag

SEQ ID NO: 141 - protein, 26 aa, Artificial Sequence
  synthetic
QHLCGSHLVEALYLVCGERGFFYTPK

SEQ ID NO: 142 - DNA, 78 bp, Artificial Sequence
  synthetic
caacacctgtgcggctcacacctggtggaagaactctacctagtgtgcggggaagaaggcttcttctacacacccaag

SEQ ID NO: 143 - protein, 26 aa, Artificial Sequence
  synthetic
QHLCGSHLVEELYLVCGEEGFFYTPK

SEQ ID NO: 144 - DNA, 78 bp, Artificial Sequence
  synthetic
ggccctggtgcaggcagcctgcagcccttggccctggaggggtccctgcagaagcgtggcattgtggaacaatgctgt

SEQ ID NO: 145 - protein, 26 aa, Artificial Sequence
  synthetic
GPGAGSLQPLALEGSLQKRGIVEQCC

SEQ ID NO: 146 - DNA, 48 bp, Artificial Sequence
  synthetic
ctgctggccctctggggacctgacccagccgcagcctttgtgaaccaa

SEQ ID NO: 147 - protein, 16 aa, Artificial Sequence
  synthetic
LLALWGPDPAAAFVNQ

SEQ ID NO: 148 - DNA, 63 bp, Artificial Sequence
  synthetic
gagagcggctgcaccgtcatcgtcatgctgaccccgctggtggaggatggtgtcaagcagtgt

SEQ ID NO: 149 - protein, 21 aa, Artificial Sequence
  synthetic
ESGCTVIVMLTPLVEDGVKQC

SEQ ID NO: 150 - DNA, 87 bp, Artificial Sequence
  synthetic
tctcagcagccctacctgcaactgcagccctttccacagcctgagctgccctatcctcagcctcagcctagctttccacctcagcag

SEQ ID NO: 151 - protein, 29 aa, Artificial Sequence
  synthetic
SQQPYLQLQPFPQPELPYPQPQPSFPPQQ

SEQ ID NO: 152 - DNA, 63 bp, Artificial Sequence
  synthetic
gaggagcagtacaacagcacctacagagtggtgagcgtgctgaccgtgctgcaccaggactgg

SEQ ID NO: 153 - protein, 21 aa, Artificial Sequence
  synthetic
EEQYNSTYRVVSVLTVLHQDW

SEQ ID NO: 154 - DNA, 7862 bp, Artificial Sequence
  synthetic
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccatggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtgtggcatctcctcagcagagagcctgaagatatctcaagctgtccatgcagcacatgcagaaatcaatgaagcaggcagagaggtggtagggtcagcagaggctggagtggatgctgcaagctgataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 155 - DNA, 8085 bp, Artificial Sequence
  synthetic
caggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccatggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtgtggcatctcctcagcagagagcctgaagatatctcaagctgtccatgcagcacatgcagaaatcaatgaagcaggcagagaggtggtagggtcagcagaggctggagtggatgctgcaagctgataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaattcgagctcgctagctaacccctatcacaattagcattaacgatcccctatcacaattagcattaaaccggtcccctatcacaattagcattaatcaccccctatcacaattagcattaacccggggtaaaacccatggaattcagttctcacgataacccatggaattcagttctcaacgcgtaacccatggaattcagttctcatcacaacccatggaattcagttctcaggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcacaaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcc

SEQ ID NO: 156 - DNA, 9213 bp, Artificial Sequence
  synthetic
aaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcgaattcctgcagcccggtgcatgactaagctagctcagttagcctcccccatctcccctagaggatccccctgttccacctcttgaaggctatgtaggccacaaggcccacaaccacagcagccaggatggagcaatagacagggatgaggttgtcggtggtgcctcgggtcaccacgggctgggagctgcccatcactgtggtcaccacacctgccaccgtgctggctatgaggtcttgttctggaggtgcctcaggctcctgggtgctgggggctgtgctgtccgagccctctgggggtgtggaccgtgtaatccaacggccagggatctcctcgcactcggcgtcggcccagcgtgtgcactcgcggagctggcgctcggtgtcctcgcacacggtgcagggcaggcacgggtccacgtggttggcctcgtcggaatacgtgccgtcggggcactcctcgcacacggtgttctgcttgtcctggcaggagaacacgaggcccgagcccgcctcgcacacgcggcacgcctcgcagcgcccagtcgtctcatcctggtagtagccgtaggcgcagcggcacacggcgtcgtcggcctccacgcacggcgccgacatgctctggagccccacgcactcggtgcacggcttgcacggctcggtcgcgctcaccacgtcggagaacgtcacgctgtccaggcagggctcacacacggtctggttggctccacaaggctgggccacaccctcgcccaggttgcaggctttgcagcactcaccgctgtgtgtgtacaggcctgtggggcatgcctccttggcacctccaagggacacccccagaagcagcaacagcagcaggcgcggcccgtccatggcgcggccggtggcacctgcccccatcgcccgcctcccgcggcagcgctcgacttccagctcggtccgctttgcggactgatggggctgcgctgcgctgcgctccagcgccccccctgcccgccggagctggccgcggcccgaattccgcggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggtcgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaagcggccgcgtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctagagcctcttccgcgtcttcgccttcccgggtcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcac

SEQ ID NO: 157 - DNA, 9109 bp, Artificial Sequence
  synthetic
aaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcgaattcctgcagcccggtgcatgactaagctagcagttcagttccggatcttcatggtcatgtaggcctcgatgtagttgatgaagatgtcgaactcgctcatggccttgtagatgcccttttcctgcagcttgttgaaggcgtttttgacctgttccacggccttgctcttgttctcgcagggcagaaatctgtggcaccgcctcagccgcagccgcagggttttcaggttctcgcccaggctgttcacgtgggccttgatgtcggggtcctggttctcggcctggggcatcacttcttccaggtagaactggatcatctcgctcagggcctggcagcccaggtagcccttgaaatcttccagcaggctctctttcagcagcaggttgtccagctggtccttcatctggaagaatgttttcactctgctgaaggcgtccctcaggtcccgcagcatgttgggcaggttgccggggaagtgggtgcagctgttctcgctctgggtgccctggccaggagaggctctgacgccggtcagcagcaccaggcagcacagcagggcggagctgtgcatagtcggtccgctttgcggactgatggggctgcgctgcgctgcgctccagcgccccccctgcccgccggagctggccgcggcccgaattccgcggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccatggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtgcatgttggtgctgttgcctgatgaagtctcaggccttgagcagcttgagagtataatcaactttgaaaaactgactgaatggaccagttctaacgttatggaagagaggaagatcaaagtgtacttacctcgcatgaagatggaggaaaaatacaacctcacatctgtcttaatggctatgggcattactgacgtgtttagctcttcagccaatctgtctggcatctcctcagcagagagcctgaagatatctcaagctgtccatgcagcacatgcagaaatcaatgaagcaggcagagaggtggtagggtcagcagaggctggagtggatgctgccacctgataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctagagcctcttccgcgtcttcgccttcccgggtcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcac

SEQ ID NO: 158 - DNA, 9577 bp, Artificial Sequence
  synthetic
aaatttcacaaataaagcatttttttcactgcattctagttgtggtttgtccaaactcatcaatgtatcttatcatgtctggctctagctatcccgcccctaactccgcccagttccgcccattctccgccccatggctgactaattttttttatttatgcagaggccgaggccgcctcggcctctgagctattccagaagtagtgaggaggcttttttggaggcctaggcttttgcgtcgagacgtacccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcccaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctggagctgcaagcttggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccggggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtggcgcccgaacagggacctgaaagcgaaagggaaaccagagctctctcgacgcaggactcggcttgctgaagcgcgcacggcaagaggcgaggggcggcgactggtgagtacgccaaaaattttgactagcggaggctagaaggagagagatgggtgcgagagcgtcagtattaagcgggggagaattagatcgcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaccaccgcacagcaagcggccgctgatcttcagacctggaggaggagatatgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcctcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagattggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatctcgacggtatcggttaacttttaaaagaaaaggggggattggggggtacagtgcaggggaaagaatagtagacataatagcaacagacatacaaactaaagaattacaaaaacaaattacaaaaattcaaaattttatcgatcacgagactagcctcgagagatctgatcataatcagccataccacatttgtagaggttttacttgctttaaaaaacctcccacacctccccctgaacctgaaacataaaatgaatgcaattgttgttgttaacttgtttattgcagcttataatggttacaaataaggcaatagcatcacaaatttcacaaataaggcatttttttcactgcattctagttttggtttgtccaaactcatcaatgtatcttatcatgtctggatctcaaatccctcggaagctgcgcctgtcatcgaattcctgcagcccggtgcatgactaagctagcagtctaaggccaactcagaagagctttctcggttgtatctttcacactccttaggaaagtcatgggattcgtacccccagtccctctgctttccacatttgagggctcttccgacttgtcgccatcagtgggcttcttcttcgaaggtttcataatgtaagtatctactattgcgaggtggaactttctcacagagaccagaccattcacacactcgttataagctttcgtcaagtcttcattgtgtcttgaaatgacaaactcacggactgggggagctgactctaagaagaaaaggaagttccggtgggctggaggcatgtactctctcatttcctggaggaattctgcaggagattctttgccagcctcgtgttttattcccagaaggacatcaagactctggaagatgctgctctggcctgcactgccccctgaaaacatttttggggtgtcccagaccccctcatacagcagaccttctggcagcttggagctgcatttccagccagacagatatatgcggagaacgtggaaaaacgtgtctgggtccacaaagtcacgcatcctcttaaaaatttccttggctttctccagactggtagctatgtcgtgcagtgccttttccaatgctttcaggtcttgacgctctactgcactggatacagtggggattgctttgattgcaggagaagctgcgatttccaccaatagagagacgaggaagaagcccttgtcgcagtccccaccaggaaatgagaacagaatgtccatgttctcgtatgtcatgggcccattggggtcctttttcttccagtttgccaggacacagtctgcataagacagaataggaggcaggcccaacttctctgagagctcgcagtagggaacagcaatattgcggggcagcacctttcgaacatcgtcatcccctcggttccacacatacgccatggtgatgtaccccagggccaggtgtgccaggcgctgtaacctgtgtcctctcagtccgtccgtgctcagtgtgggcagcttttcaacttcttctcgaagctgcccgttctcaatcagcacaggcagatttctagccacaaggacccaggggctgtatgcgtcgggcagctccaccagtggatgtggtagagcaaagcccacatcttcatctatgtggtggtcttcaaggattcttctagaaccttctgtaggagatattttactgagtgccatagtcggtccgctttgcggactgatggggctgcgctgcgctgcgctccagcgccccccctgcccgccggagctggccgcggcccgaattccgcggaggctggatcggtcccggtgtcttctatggaggtcaaaacagcgtggatggcgtctccaggcgatctgacggttcactaaacgagctctgcttatataggcctcccaccgtacacgcctaccctcgagaagcttgatatcgaattcccacggggttggggttgcgccttttccaaggcagccctgggtttgcgcagggacgcggctgctctgggcgtggttccgggaaacgcagcggcgccgaccctgggtctcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcggggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccatggatgaccaacgcgacctcatctctaaccatgaacagttgcccatactgggcaaccgccctagagagccagaaaggtgcagccgtggagctctgtacaccggtgtctctgtcctggtggctctgctcttggctgggcaggccaccactgcttacttcctgtaccagcaacagggccgcctagacaagctgaccatcacctcccagaacctgcaactggagagccttcgcatgaagcttccgaaatctgccaaacctgtgagccagatgcggatggctactcccttgctgatgcgtccaatgtccatggataacatgctccttgggcctgtgaagaacgttaccaagtacggcaacatgacccaggaccatgtgatgcatctgctcacgaggtctggacccctggagtacccgcagctgaaggggaccttcccagagaatctgaagcatcttaagaactccatggatggcgtgaactggaagatcttcgagagctggatgaagcagtggctcttgtttgagatgagcaagaactccctggaggagaagaagcccaccgaggctccacctaaagagccactggacatggaagacctatcttctggcctgggagtgaccaggcaggaactgggtcaagtcaccctgtgtggcatctcctcagcagagagcctgaagatatctcaagctgtccatgcagcacatgcagaaatcaatgaagcaggcagagaggtggtagggtcagcagaggctggagtggatgctgcaagctgataagtcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctagagcctcttccgcgtcttcgccttcccgggtcgagctcggtacctttaagaccaatgacttacaaggcagctgtagatcttagccactttttaaaagaaaaggggggactggaagggctaattcactcccaacgaagacaagatctgctttttgcttgtactgggtctctctggttagaccagatctgagcctgggagctctctggctaactagggaacccactgcttaagcctcaataaagcttgccttgagtgcttcaagtagtgtgtgcccgtctgttgtgtgactctggtaactagagatccctcagacccttttagtcagtgtggaaaatctctagcagtagtagttcatgtcatcttattattcagtatttataacttgcaaagaaatgaatatcagagagtgagaggaacttgtttattgcagcttataatggttacaaataaagcaatagcatcac

SEQ ID NO: 159 - DNA, 537 bp, Artificial Sequence
  synthetic
atgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactga

SEQ ID NO: 160 - protein, 178 aa, Artificial Sequence
  synthetic
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 161 - DNA, 1212 bp, Artificial Sequence
  synthetic
atggcccatgccatggaaaacagctggaccatcagcaaagagtaccacatcgacgaggaagtgggcttcgccctgcctaatcctcaagagaacctgcctgacttctacaacgactggatgtttatcgccaaacatctgcccgacctgatcgagagcggccagctgagagaaagagtggaaaagctgaacatgctgagcatcgaccacctgaccgaccacaagtctcagagactggccagactggtgctgggctgtatcaccatggcctacgtgtggggaaaaggccatggcgacgtgcggaaagtgctgcccagaaatatcgccgtgccttactgccagctgtccaagaagctggaactgcctcctatcctggtgtacgccgattgcgtgctggccaactggaagaagaaggaccccaacaagcccctgacctacgagaacatggacgtgctgtttagcttccgcgacggcgattgcagcaagggattcttcctggtgtccctgctggtggaaatcgccgctgcctctgccatcaaagtgatccccaccgtgttcaaggccatgcagatgcaagagcgggacaccctgctgaaggccctgctggaaattgcctcctgcctggaaaaagccctccaggtgttccaccagatccacgaccacgtgaaccccaaggccttcttcagcgtgctgcggatctatctgtctggctggaagggcaatccccagctgtctgacggcctggtgtatgaaggcttctgggaagatcccaaagagttcgctggcggctctgccggacagtctagtgtgttccagtgcttcgatgtgctgctgggcatccagcaaacagccggcggaggacatgctgctcagtttctgcaagacatgcggcggtacatgcctccagctcaccggaactttctgtgcagcctggaaagcaaccccagcgtgcgggaattcgtgctgtctaaaggcgacgccggactgagagaagcctacgatgcctgtgtgaaggctctggtgtctctgcggagctaccacctccagatcgtgaccaagtacattctgatccccgccagccagcagcctaaagagaacaagaccagcgaggacccctccaagctggaagcaaaaggcacaggcggaaccgatctgatgaacttcctgaaaaccgtgcggtccaccaccgagaagtctctgctgaaagagggctga

SEQ ID NO: 162 - protein, 403 aa, Artificial Sequence [sequence omitted]

SEQ ID NO: 163 - DNA, 4346 bp, Artificial Sequence
  synthetic
atgggcgcgagaaactccgtcttgtcagggaagaaagcagatgaattagaaaaaattaggctacgacccggcggaaagaaaaagtacatgttgaagcatgtagtatgggcagcaaatgaattagatagatttggattagcagaaagcctgttggagaacaaagaaggatgtcaaaaaatactttcggtcttagctccattagtgccaacaggctcagaaaatttaaaaagcctttataatactgtctgcgtcatctggtgcattcacgcagaagagaaagtgaaacacactgaggaagcaaaacagatagtgcagagacacctagtggtggaaacaggaacagcagaaactatgccaaaaacaagtagaccaacagcaccatctagcggcagaggaggaaattacccagtacaacaaataggtggtaactatgtccacctgccattaagcccgagaacattaaatgcctgggtaaaattgatagaggaaaagaaatttggagcagaagtagtgccaggatttcaggcactgtcagaaggctgcaccccctatgacattaatcagatgttaaattgtgtgggagaccatcaagcggctatgcagattatcagagatattataaatgaggaggctgcagattgggacttgcagcacccacaaccagctccacaacaaggacagcttagggagccgtcaggatcagatattgcaggaacaactagttcagtagatgaacaaatccagtggatgtacagacaacagaaccccataccagtaggcaacatttacaggagatggatccaactggggttgcaaaaatgtgtcagaatgtataacccaacaaacattctagatgtaaaacaagggccaaaagagccatttcagagctatgtagacaggttctacaaaagcttaagagcagaacaaacagatgcagcagtaaagaattggatgactcaaacactgctgattcaaaatgctaacccagattgcaagctagtgctgaaggggctgggtgtgaatcccaccctagaagaaatgctgacggcttgtcaaggagtagggggaccaggacagaaggctagattaatggcagaagccctgaaagaggccctcgcaccagtgccaatcccttttgcagcagcccagaagaggggaccaagaaagccaattaagtgttggaattgtgggaaggagggacactctgcaaggcaatgcagagccccaagaagacagggatgctggaaatgtggaaaaatggaccatgttatggccaaatgcccagacagacaggcgggttttttaggccttggtccatggggaaagaagccccgcaatttccccatggctcaagtgcatcaggggctgacgccaactgctcccccagaggacccagctgtggatctgctaaagaactacatgcagttgggcaagcagcagagagaaagcagagagaagccttacaaggaggtgacagaggatttgctgcacctcaattctctctttggaggagaccagtagtcactgctcatattgaaggacagcctgtagaagtattattggatacaggggctgatgattctattgtaacaggaatagagttaggtccacattataccccaaaaatagtaggaggaataggaggttttattaatactaaagaatacaaaaatgtaaaaatagaagttttaggcaaaaggattaaagggacaatcatgacaggggacactccgattaacatttttggtaggaatttgctaacagctctggggatgtctctaaatcttcccatagctaaggtagagcctgtaaaagtcaccttaaagccaggaaaggttggaccaaaattgaagcagtggccattatcaaaagaaaagatagttgcattaagagaaatctgtgaaaagatggaaaaggatggtcagttggaggaagctcccccgaccaatccatacaacacccccacatttgccataaagaaaaaagataagaacaaatggagaatgctgatagattttagggaactaaatagggtcactcaggactttacagaagtccaattaggaataccacaccctgcaggactagcaaaaaggaaaaggattacagtactggatataggtgatgcatatttctccatacctctagatgaagaatttaggcagtacactgcctttactttaccatcagtaaataatgcagagccaggaaaacgatacatttataaggttctgcctcagggatggaaggggtcaccagccatcttccaatacactatgagacatgtgctagaacccttcaggaaggcaaatccagatgtgaccttagtccagtatatggatgacatcttaatagctagtgacaggacagacctggaacatgacagggtagttttacagctaaaggaactcttaaatagcatagggttctctaccccagaagagaaattccaaaaagatcccccatttcaatggatggggtacgaattgtggccgacaaaatggaagttgcaaaagatagagttgccacaaagagagacctggacagtgaatgatatacagaagttagtaggagtattaaattgggcagctcaaatttatccaggtataaaaaccaaacatctctgtaggttaattagaggaaaaatgactctaacagaggaagttcagtggactgagatggcagaagcagaatatgaggaaaataagataattctcagtcaggaacaagaaggatgttattaccaagaaggcaagccattagaagccacggtaataaagagtcaggacaatcagtggtcttataaaattcaccaagaagacaaaatactgaaagtaggaaaatttgcaaagataaagaatacacataccaatggagttagactattagcacatgtaatacagaaaataggaaaggaagcaatagtgatctggggacaggtcccaaaattccacttaccagttgagagggatgtatgggaacagtggtggacagactattggcaggtaacctggataccggagtgggattttatctcaacgccaccactagtaagattagtcttcaatctagtgaaggaccctatagagggagaagaaacctattatacagatggatcatgtaataaacagtcaaaagaagggaaagcaggatatatcacagataggggcaaagacaaagtaaaagtgttagaacagactactaatcaacaagcagaattagaagcatttctcatggcattgacagactcagggccaaagacaaatattatagtagattcacaatatgttatgggaataataacaggatgccctacagaatcagagagcaggctagttaaccaaataatagaagaaatgattaaaaagtcagaaatttatgtagcatgggtaccagcacacaaaggtataggaggaaaccaagaaatagaccacctagttagtcaggggattagacaagttctcttcttggaaaagatagagccagcacaagaagaacatgataaataccatagtaatgtaaaagaattggtattcaaatttggattacccagaatagtggccagacagatagtagacacctgtgataaatgtcatcagaaaggagaagctatacatgggcaggtaaattcagatctagggacttggcaaatggactgtacccatctagaaggaaaaatagtcatagttgcagtacatgtagctagtggattcatagaagcagaagtaattccacaagagacaggaagacagacagcactatttctgttaaaattggcaggcagatggcctattacacatctacacacagataatggtgctaactttgcctcgcaagaagtaaagatggttgcatggtgggcagggatagagcacacctttggggtaccatacaatccacagagtcagggagtagtggaagcaatgaatcaccacctgaaaaatcaaatagatagaatcagggaacaagcaaattcagtagaaaccatagtattaatggcagttcattgcatgaattttaaaagaaggggaggaataggggatatgactccagcagaaagattaattaacatgatcactacagaacaagaaatacaatttcaacaatcaaaaaactcaaaatttaaaaattttcgggtctattacagagaaggcagagatcaactgtggaagggacccggtgagctattgtggaaaggggaaggagcagtcatcttaaaggtagggacagacattaaggtagtacccagaagaaaggctaaaattatcaaagattatggaggaggaaaagaggtggatagcagttcccacatggaggataccggagaggctagagaggtggcatag

SEQ ID NO: 164 - protein, 506 aa, Artificial Sequence
  synthetic
MGARNSVLSGKKADELEKIRLRPGGKKKYMLKHVVWAANELDRFGLAESLLENKEGCQKILSVLAPLVPTGSENLKSLYNTVCVIWCIHAEEKVKHTEEAKQIVQRHLVVETGTAETMPKTSRPTAPSSGRGGNYPVQQIGGNYVHLPLSPRTLNAWVKLIEEKKFGAEVVPGFQALSEGCTPYDINQMLNCVGDHQAAMQIIRDIINEEAADWDLQHPQPAPQQGQLREPSGSDIAGTTSSVDEQIQWMYRQQNPIPVGNIYRRWIQLGLQKCVRMYNPTNILDVKQGPKEPFQSYVDRFYKSLRAEQTDAAVKNWMTQTLLIQNANPDCKLVLKGLGVNPTLEEMLTACQGVGGPGQKARLMAEALKEALAPVPIPFAAAQKRGPRKPIKCWNCGKEGHSARQCRAPRRQGCWKCGKMDHVMAKCPDRQAGFLGLGPWGKKPRNFPMAQVHQGLTPTAPPEDPAVDLLKNYMQLGKQQRESREKPYKEVTEDLLHLNSLFGGDQ

(19) United States
(12) Patent Application Publication
(10) Pub. No.: US 2021/0277354 A1
(43) Pub. Date: Sep. 9, 2021

(54) PRODUCTION OF ENGINEERED DENDRITIC CELLS AND USES THEREOF

(71) Applicants: FONDAZIONE TELETHON, Roma (IT); FONDAZIONE CENTRO SAN RAFFAELE, Milano (MI) (IT); OSPEDALE SAN RAFFAELE S.R.L., Milano (MI) (IT)

(72) Inventors: Andrea ANNONI, Milano (MI) (IT); Silvia Adriana GREGORI, Milano (MI) (IT)

(73) Assignees: FONDAZIONE TELETHON, Roma (IT); FONDAZIONE CENTRO SAN RAFFAELE, Milano (MI) (IT); OSPEDALE SAN RAFFAELE S.R.L., Milano (MI) (IT)

(21) Appl. No.: 17/253,301

(22) PCT Filed: Jun. 19, 2019
(86) PCT No.: PCT/EP2019/066284

§ 371 Date: Dec. 17, 2020

(30) Foreign Application Priority Data
Jun. 19, 2018 (EP) 18178595.7

(51) Int. Cl. C12N 5/0784; C12N 5/0783; A61K 35/15; C12N 15/85; A61K 45/06

(52) U.S. Cl. CPC C12N 5/064; C12N 5/0637; A61K 35/15; C12N 2510/00; A61K 45/06; C12N 2502/1121; C12N 15/85

(57) ABSTRACT

The present disclosure relates to a genetically modified dendritic cell or precursor thereof expressing at least one anti-gen-derived peptide and at least one immuno-modulatory molecule, its medical use and method of preparation. The invention also relates to an in vitro method to produce IL-10-producing CD49b+LAG-3+ Tr1 cells or antigen-specific FOXP3+ T cells and relative medical uses and pharmaceutical compositions.

### TECHNICAL FIELD

The present disclosure relates to a genetically modified dendritic cell or precursor thereof expressing at least one antigen-derived peptide and at least one immuno-modulatory molecule, its medical use and method of preparation. The invention also relates to an in vitro method to produce IL-10-producing CD49b⁺ D+LAG-3+Tr1 cells or antigen-specific FOXP3⁺ T cells and relative medical uses and pharmaceutical compositions.

### BACKGROUND ART

Identification of novel approaches designed to selectively control antigen(Ag)-specific pathogenic T cell responses and promote/restore tolerance in T-cell mediated diseases represents one of the ambitious goals for the management of autoimmune disease and organ transplantation in humans. On this line, a new version of vaccination, also called “inverse vaccination” or “tolerogenic vaccination”, aims at inducing or restoring an immunological state of unresponsiveness, which can be either towards foreign Ags (i.e. protein therapeutics, allergens, or transgenes) or autoAgs (1). The overall goal of tolerogenic strategies is to dampen the adverse response, through deletion/inhibition/deviation of Ag-specific Teff cells, and to support the induction and/or expansion of Ag-specific T regulatory cells (Tregs) either the forkhead box P3 (FOXP3)-expressing Tregs (FOXP3⁺ Tregs) (2) or the IL-10-producing T regulatory type 1 (Tr1) cells (3). A number of different approaches have been proposed as inverse vaccination:
i) non-Ag-specific immunotherapies with monoclonal antibodies targeting different cell populations (i.e. anti-CD3, anti-CD20, anti-CD52, CTLA-4Ig) or pro-inflammatory cytokines (i.e. anti-TNFα, anti-IL-1β), or with immunomodulatory compounds (i.e. Rapamycin, Mycophenolate Mofetil),
ii) Ag-specific immunotherapies with autoAgs or allergens.

As actors of tolerogenic strategies, regulatory cells have been proposed as cell therapy tools. Growing evidence indicates that different subsets of dendritic cells (DC), either naturally arising or experimentally induced, play a critical role in the maintenance of tissue homeostasis and in promoting tolerance (reviewed in (4-7)), thus acting as regulatory cells. The regulatory capacity of DC depends on their immature state, and can be induced by immunosuppressive mediators, genetic manipulation or signals from other immune cells. Tolerogenic DC (tolDC) present Ag and prime Ag-specific T cells and can also induce Ag-specific Tregs (8). A better understanding of the biology of tolDC and of the mechanisms regulating their induction, activity, and plasticity, together with the development of protocols suitable for the generation of tolDC in vitro, opened the possibility to translate their use as immunotherapy in immune-mediated diseases (8-12). DC represent the tolerogenic cells of choice to fulfill the goal of promoting/restoring Ag-specific tolerance, since they i) promote Ag-specific Tregs; ii) modulate Ag-specific pathogenic T cells; iii) generate a tolerogenic microenvironment enriched in anti-inflammatory mediators that sustains the maintenance of long-term Ag-specific unresponsiveness. The proof-of-principle clinical trials, so far completed, demonstrated the safety and feasibility of tolDC-based immunotherapy in preventing graft rejection after organ transplantation and in autoimmune diseases (10, 11, 13-15). However, stability of infused DC and the maintenance of their tolerogenic properties remain open issues for improving the safety and the efficacy of a successful DC-based cell therapy.

Optimal tolDC should present Ag in a not activated state or in a microenvironment enriched in anti-inflammatory cytokines or inhibitory molecules. To stabilize these conditions, the inventors propose the use of novel strategies based on state-of-the-art lentiviral vector (LV) technology that will ensure the generation of stable and efficacious tolerogenic DC. Lentiviral vectors (LVs) transduce human DC precursors (16) and induce strong and durable anti-tumor T cell responses (17). Moreover, LV-mediated DC transduction does not result in major changes in the state of DC activation (18), supporting the possibility to exploit LV-mediated stable and efficient Ag presentation to generate immunogenic or tolerogenic DC. Thus far, LVs has been used to genetically modify DC for immunogenic DC-based therapies. DC transduced with LV encoding for tumor-associated Ags generate tumor-specific CD8⁺ T cells (17). Priming of CD4⁺ T cells by LV-transduced DC occurs only if the LV-encoded Ag has access to an MHC class II presentation pathway. LV encoding for the invariant chain (Ii) fused with ovalbumin (OVA) (LV.IiOVA) in vivo injected transduced DC that acquired the ability to present encoded OVA in the contest of MHC class II and promote OVA-specific CD4⁺ T cells (19). Direct in vivo LV administration to transduce DC offers some advantages: it does not require cell manipulation, and the vector itself triggers acute inflammation providing an adjuvant effect; however, it cannot offer high specificity of cell targeting. Conversely, the in vitro LV-mediated DC transduction can significantly improve safety by minimizing off-target transduction and by the limited life span of transferred cells. Moreover, administering in vitro generated LV-transduced DC allows repetitive cell administrations. A plethora of agents have been employed to differentiate in vitro human tolDC (20). To define the optimal tolerogenic DC to be used in vivo, it was recently reported a comparative analysis of different subpopulations of in vitro differentiated tolDC examining their stability, cytokine production profile, and suppressive activity (20, 21). The results indicated that IL-10-modulated mature DC are the best-suited cells for tolerogenic DC-based therapies. The inventors' group contributed to the identification of IL-10 as key factor for promoting the differentiation of potent tolerogenic DC, and described a subset of cells, named DC-10, that can be induced in vitro from peripheral blood monocytes in the presence of IL-10 and are characterized by the ability to secrete high amounts of IL-10. DC-10 are mature myeloid cells expressing a set of immunomodulatory molecules including HLA-G, ILT3, and ILT4, which render them potent inducers of Ag-specific Tr1 cells in vitro (22, 23). DC-10 are stable cells since they maintain their tolerogenic activity even upon activation (24). Interestingly, stimulation of allergen-specific T cells with autologous DC-10 promotes their conversion into IL-10-producing suppressive T cells (25), indicating that DC-10 represent a good candidate to convert effector T cells into Tregs. The over-expression of IL-10 converted murine bone marrow derived DC in tolDC that upon in vivo transfer prevent allergic contact dermatitis (26). Alternative candidates to confer tolerogenic properties to DC is the induction of indoleamine 2,3-dioxygenase (IDO1), a tryptophan catabolizing enzyme, regulator of immunity in several pathological conditions. Expression of IDO have been promoted by several means in antigen-presenting cells, including plasmacytoid and myeloid DC ((27), WO 2013/040552, WO2018037108, and WO2017192786. Overall, these methods do not promote stable and long-lasting overexpression of IDO in treated cells. One of the major goals of the clinicians is to identified alternative treatments to prevent graft rejection after organ transplantation. The improvements of immunosuppressive therapy treatments used to prevent rejection after allogenic organ transplantation shows benefit in limiting acute rejection, however the side effects associated to the long-term immunosuppressive regimens (see approved drugs Table 1 below) represent one of the major causes of chronic graft failure. Standard immunosuppressive regimens are effective. However, they require long-term treatments, which are associated with a number of side effects, and the current life expectancy of transplanted-patients including is kindey transplanted patients still significantly short compared to that of the general population (van Sandwijk M S et al., Neth J Med. 2013). Immunosuppressive treatments are administered every day leading to an annual cost 14K$.

TABLE 1
Approved drugs

| | | | |
|---|---|---|---|
| Mycophenolate | Immunosuppressive | Inhibits inosine | Decreases B and T cell |
| mofetil | (Anti-proliferative) | monophosphate | proliferation |
| | | dehydrogenase | |
| Rapamycin | Immunosuppressive | Blocks cell cycle at | Decreases B and T cell |
| (Sirolimus) | (Anti-proliferative) | G1/S phase | proliferation, spears |
| | | | Tregs, and decreases |
| | | | antibody production |
| Everolimus | Immunosuppressive | Same as | Same as Rapamycin |
| (derivative of | (Anti-proliferative) | Rapamycin | (Sirolimus) |
| Sirolimus) | | (Sirolimus) | |
| Leflunomide | Immunosuppressive | Blocks | Decreases activated |
| | (Anti-proliferative) | dihydroorotate | lymphocyte proliferation |
| | | dehydrogenase, | and differentiation |
| | | limiting the | |
| | | production of | |
| | | uridine | |
| | | monophosphate | |
| | | (UMP) | |
| Azithioprine | Immunosuppressive | Blocks de novo | Blocks T cell activation |
| | (Anti-proliferative) | purine synthesis | |
| Methylprednisolone | Immunosuppressive | Causes | Decreases circulating T |
| | (Anti-proliferative | redistribution of T | cells and inflammatory |
| | and anti- | cells and blocks | cytokines (i.e., IL-6) |
| | inflammatory) | inflammatory | |
| | | pathways | |
| Tacrolimus (FK506) | Immunosuppressive | Causes decrease | Decreases cellular and |
| | (Anti-proliferative | in gene expression | humoral immunity |
| | and antibiotic) | | |
| Rituximab | Anti-CD20 | Antibody- | Depletes CD20⁺ B cells |
| | monoclonal antibody | dependent cellular | |
| | | cytotoxicity | |

Alternative therapies based on regulatory cell immunotherapy entered the clinical arena in the last decade, with the goal of tapering immunosuppression (28). Among them T regulatory cell (Treg)-based therapies. Thus far, up to 30 different clinical trials have been completed or are ongoing using polyclonal freshly isolated or in vitro expanded Tregs to prevent graft rejection (29). Ongoing clinical trials with Treg-based therapy demonstrated the safety of the approach and some clinical benefit. However, several open issues remain to be solved:
The potential of polyclonal in vitro expanded Tregs to mediate pan immunosuppression in vivo (30); for this reason, pre-clinical studies are ongoing to generate antigen-specific Tregs to limit this side effect;
The potential of infused Tregs to be destabilized in strong inflammatory conditions in vivo and adopt pathogenic effector T phenotype and functions, thereby possibly mediating graft rejection; The overall impact of long-lasting Tregs on hampering immunity against infections and malignancies (29).

An interesting alternative and complementary approach to Treg-based therapy is represented by the myeloid regulatory cell (MRC)-based therapies. MRC (i.e., Mreg and TolDC) exert immune regulatory effects through different mechanisms compared to Tregs, including depletion of Ag-specific effector T cells, promoting tissue repairing and regeneration process. Moreover, MRC induce Ag-specific Tregs in vivo in a physiological manner. Only few patients have been treated with MRCs (i.e., Mregs or TolDC). Thus far, published data on a small number of transplanted patients demonstrated the safety of the approach and showed that infusion of donor-derived Mregs in kidney-transplanted patients allows tapering of immunosuppressive regimen and induction of Tregs in vivo (31).

Therefore, there is still the need for cell therapy for the treatment of autoimmune diseases, inflammatory diseases, graft versus host diseases.

### SUMMARY OF THE INVENTION

Ag-presentation by immature DC is well known naturally occurring mechanism to induce peripheral immune tolerance (32) and the inventors propose to exploit immune-modulatory regulation to ensure Ag presentation by immature genetically modified DC.

In the present invention, it was surprisingly found that genetically modified dendritic cells or precursor thereof modified with a nucleic acid comprising the combination of i) a sequence encoding a chimeric protein consisting of a human invariant chain fused to at least one antigenic peptide or protein or an antigenic fragment thereof and ii) a sequence encoding at least one immuno-modulatory protein, is particularly advantageous for therapeutic applications.

The nucleic acid may also further comprise at least one miRNA target sequence.

MiRNAs are small non-coding RNAs, which negatively regulate the expression of specific target genes at post-transcriptional level (33). When miRNAs are partially complementary to the target messenger RNA (mRNA) sequences at 3′-untranslated regions (3′UTR), they reduce target mRNA stability and inhibit translation. Alternatively, when miRNAs are nearly perfectly complementary to their mRNA targets, they cleave the mRNA, triggering its wholesale destruction, therefore the lack of protein expression. MiRNAs have distinct expression profiles in different tissues and cell types, which differentially regulate transcriptional profiles of genes and cellular functions, thus providing a cell-specific and developmental stage-specific regulation of gene expression (34). MiRNAs play a crucial role in controlling many processes within the immune system including cell differentiation and homeostasis, cytokine responses, interactions with pathogens and tolerance induction. DC development, differentiation and function are regulated by a specific expression profile of miRNAs. In particular, miR-155 and miR-146a expression is associated with DC maturation both in human and mouse (35-38). Therefore, by the insertion of 2×miR155 and 2×miR146a target sequences (miR155T.mir146aT) in the 3′ UTR region of the LV cassette encoding for the invariant chain (Ii) fused with a selected portion of the desired Ag (LV.IiAg), the inventors achieve the repression of the transgene expression, hence Ag-presentation in LV-DC which enter in the activation program.

Methods provided herein are designed to induce a tolerogenic response to the LV-encoded Ag. The efficacy of LV-mediated gene transfer into DC and their precursors offers several clinically applicable opportunities to exploit functional plasticity of DC to design specific immunotherapies both for tolerance induction in autoimmunity and transplants.

According to an embodiment of the invention, LV-IL-10 engineered DC (DC^{IL-10}) may be useful in preventing graft rejection after organ transplantation.

LV-mediated gene transfer of IL-10 in DC (DC^{IL-10}) has the potential to overcome the major limitations of Treg-based therapies and to be more effective compared to other MRCs, as it will result in a drug product that will:
induce allo-specific immunological non-responsiveness in effector T cells;
promote a self-reinforcing peripheral regulation, with the induction of allo-specific Tregs in vivo in a physiological manner;
have a limited life span in vivo (up to 14 days), overall limiting the long-lasting impact on immunity against infections and malignancies;
promote stable over-expression of IL-10 ensuring the generation of a local microenvironment enriched in IL-10, which modulates T cells, myeloid cells, and innate cells, sustaining long-term tolerance.

The present invention provides methods for inducing tolerance or suppressing an immune response to an antigen by regulatory immune cells, wherein immune cells are genetically modified by newly developed tolerogenic vectors, preferably LV encoding Ag-derived peptides or antigenic peptides, such as epitopes, that allow the expression of Ag-derived peptides or antigenic peptides and pro-tolerogenic molecules. In some embodiments, the tolerogenic cell is delivered to an individual and presentation of the Ag induces tolerance and/or suppresses immune response to the Ag. In some embodiments, the tolerogenic cells are used to promote Ag-specific Tregs in vitro, suitable for cell-based approaches.

The present invention provides a method for inducing tolerance to an Ag in an individual, the method comprising the generation of engineered immune cells with vectors, preferably lentiviral vectors (LV), to confer the expression of Ag-derived peptides (epitopes) and pro-tolerogenic molecules. The inventors have developed several LV-based gene transfer tools that allow coordinated expression of two transgenes (bidirectional (bd)LV (39, 40), WO2004094642 incorporated by reference) and/or targeted transgene expression to a specific cell subset by exploiting post-transcriptional regulation mediated by endogenous miRNA (miRNA regulated LV (41, 42) WO2010125471 incorporated by reference). Moreover, the inventors generated LV encoding for the invariant chain (Ii) fused to an Ag under the control of the Phosphoglycerate kinase 1 (PGK) ubiquitous promoter (PGK.Ii-Ag) (43), which ensures stable presentation of the encoded Ag in the context of MHC class I as an endogenous Ag, but also allows Ag processing and presentation in the contest of MHC class II as an exogenous Ag, leading to both CD4⁺ and CD8⁺ T cell stimulation.

The present invention is advantageous in that
vector-mediated transduction of DC precursors or DC allows stable expression of encoded peptides, which renders resulting DC more effective in presenting Ag to T cells;
The inclusion of miRNA target sequences allows negative post-transcriptional regulation of the encoded Ag, limiting Ag presentation at immature stage by DC-Ag.miRNA and preventing Ag presentation in an inflammatory microenvironment;
Stable over-expression of IDO or IL-10 mediated by vector(s) ensures Ag-presentation in a microenvironment enriched in IDO or IL-10;
human DC precursors are stably transduced with vectors, in particular LVs;
a population of engineered DC with multiple specificity may be used;
Different engineered DC may be combined to maximize the tolerogenic activity;
DC-IL-10/Ag and DC-Ag.miRNA-T (or DC-Ag.miRNA, i.e. containing a miRNA target sequence) promote differentiation of Ag-specific Tr1 cells in vivo;
Engineered DC are short-term living cells allowing multiple DC injections;
High versatile generation of Ii-Ag constructs to drive specific Ag expression.

According to the present invention, a strong inhibition of T effector cells and/or a strong activation of T regulatory cells is produced, as exemplified with three different approaches.

According to a preferred embodiment of the LVs described herein:
the promoter may be ubiquitous (such as PGK)
the vector may be bidirectional when the approach is DC-IL-10/Ag or DC-IDO/Ag.

According to a preferred embodiment, the clinical protocol based on the use of the tolerogenic DC of the present invention would provide that:
the modified autologous/allogenic DC are administered to the patient through one to multiple infusions to re-establish/induce a stable tolerance to the specific antigen;
the autologous/allogenic DC are modified through the transduction with single or a mixture of LVs coding for different fragments of the antigen (according to known antigen libraries) and/or different pro-tolerogenic molecules to re-establish/induce a tolerogenic response that covers multiple-specificity.

According to another preferred embodiment the DC-IL-10/Ag could be contemporaneously used in vitro to generate T regulatory type 1 cells (Tr1), according to the protocol described in WO2007131575 (incorporated by reference), that are specific for the antigen. Such antigen-specific Tr1 cells could be purified in vitro according to the protocol described in WO2013192215 (incorporated by reference) and then infused in the patient in combination with the infusion of the modified tolerogenic DC of the present invention in order to maximize the tolerogenic response toward the antigen.

Then the present invention provides a genetically modified dendritic cell or a precursor cell thereof modified with a nucleic acid construct said construct comprising:
a nucleic acid sequence a) encoding a chimeric protein consisting of a human invariant chain fused to at least one antigenic peptide or protein or an antigenic fragment thereof, said sequence a) being operatively linked to a first promoter and optionally to a first transcription regulatory sequence and
a nucleic acid sequence b) encoding at least one immuno-modulatory protein, said sequence b) being optionally operatively linked to a second promoter and optionally linked to a second transcription regulatory sequence.

The precursor cell is a precursor cell of a dendritic cell and is also genetically modified.

Then the genetically modified dendritic cell or precursor thereof constitutively expresses at least one antigen-derived peptide (or antigenic peptide or protein or an antigenic fragment thereof) and at least one immuno-modulatory molecule. Such modified cell presents at least one molecule on the cell surface or intracellularly or produces and/or secretes at least one molecule. The modification may be introduced by transduction, transformation, or electroporation.

The first promoter and the second promoter may be the same or different.

Promoters include promoters of the family of phosphoglycerated kinases 1 (PGK), Cytomegalovirus (CMV), spleen focus forming virus (SSPV), human elongation factor 1a (EF1α), myeloid related protein 8 (MRP8), myeloid-specific promoter (MSP), CAG promoter composed of CMV immediate early enhancer linked to chicken β-actin promoter, synthetic myeloid-specific promoted (146gp61), mouse mammary tumor virus (MMTV), CD11b, protein-tyrosine kinase (c-Fes), Cytochrome B-245 Beta Chain (CYBB), and Receptor Tyrosine Kinase (TEK).

The first transcription regulatory sequence and second transcription regulatory sequence may be the same or different.

The antigenic peptide or protein or an antigenic fragment thereof also refers to antigenic peptide or antigenic protein variants.

The nucleic acid may also comprise a sequence coding for the immunodominant peptide and its variable flanking regions, each of said flanking regions consisting of 5 to 10 amino acids.

Preferably said sequence a) further comprises at its 3′ end at least one miRNA target sequence.

Preferably said nucleic acid construct further comprises a sequence encoding Vpx.

Preferably said nucleic acid construct further comprises a sequence encoding a marker.

Preferably a selectable marker, preferably the marker is GFP, ΔNGFR, ΔCD19

Preferably the human invariant chain is Iip33, Iip41, Iip35 or Iip43.

Preferably said antigenic peptide or protein or antigenic fragment thereof is derived from an auto-antigen and/or a non-harmful antigen and/or an allergen.

Preferably said antigenic peptide or protein or antigenic fragment thereof is selected from the group of immunodominant peptides as described in Table 2 or variants thereof. The variants are antigenic variants.

In a preferred embodiment said immuno-modulatory protein is selected from the group consisting of: IL-10, indoleamine 2,3-dioxygenase (IDO), PDL-1, PDL-2, ILT-3, ILT-4, HO-1, ICOS-L Gal9, HVME, HLA-G, HLA-E, IL-35, TGF-b, CTLA-4Ig, PGE2, TNFRs, Arg1, preferably IL-10, indoleamine 2,3-dioxygenase (IDO) or a mixture thereof.

In a preferred embodiment the at least one miRNA target sequence is selected from the group targeting: miR-15a, miR-16-1, miR-17, miR-18a, miR-19a, miR-20a, miR-19b-l, miR-21, miR-29a, miR-29b, miR-29c, miR-30b, miR-31, miR-34a, miR-92a-l, miR-106a, miR-125a, miR-125b, miR-126, miR-142-3p, miR-146a, miR-150, miR-155, miR-181a, miR-223 and miR-424, preferably miR155, miR146a or a mixture thereof, preferably said miRNA target sequence is repeated. Preferably the miR155 target sequence is repeated twice and the miR146a target sequence is repeated twice.

Preferably the genetically modified dendritic cell or a precursor cell thereof is a cell that displays at least one of the following properties: modulates CD4⁺ and CD8⁺ T cell responses; modulates antigen-specific CD4⁺ and CD8⁺ T cell proliferation in vitro and/or in vivo; favors the generation of regulatory DC; favors the expansion of antigen-specific Tr1 and/or FOXP3⁺ Treg cells, is tolerogenic, presents antigen in the context of both MHC class I and class II.

Preferably said nucleic acid construct is inserted into a vector, preferably a lentiviral vector, more preferably a mono- or bi-directional vector.

In a preferred embodiment the genetically modified dendritic cell or a precursor cell thereof according to the invention is for medical use, preferably for use for the prevention and/or treatment of a condition selected from the group consisting of: graft versus host disease, organ rejection, autoimmune disease, allergic disease, inflammatory or auto-inflammatory disease, immune response induced by gene therapy.

Preferably the autoimmune disease is selected from the group consisting of: type 1 diabetes mellitus, autoimmune enteropathy, rheumatoid arthritis, systemic lupus erythematosus, multiple sclerosis, autoimmune myositis, psoriasis, Addison's disease, Grave's disease, Sjogren's syndrome, Hashimoto's thyroiditis, myasthenia gravis, vasculitis, pernicious anemia, celiac disease, autoimmune hepatitis, alopecia areata, pemphigus vulgaris, vitiligo, aplastic anemia, autoimmune uveitis, Alopecia Areata, Amyotrophic Lateral Sclerosis (Lou Gehrig's), Ankylosing Spondylitis, Anti-GBM Nephritis, Antiphospholipid Syndrome, Osteoarthritis, Autoimmune Active Chronic Hepatitis, Autoimmune Inner Ear Disease (AIED), Balo Disease, Behcet's Disease, Berger's Disease, Bullous Pemphigoid, Cardiomyopathy, Chronic Fatigue Immune Dysfunction Syndrome, Churg Strauss Syndrome, Cicatricial Pemphigoid, Cold Agglutinin Disease, Colitis Cranial Arteritis, Crest Syndrome, Crohn's Disease, Dego's Disease, Dermatomyositis & JDM, Devic Disease, Eczema, Essential Mixed Cryoglobulinemia, Eoscinophilic Fascitis, Fibromyalgia—Fibromyositis, Fibrosing Alveolitis, Giant Cell Arteritis, Glomerulonephritis, Goodpasture's Disease, Guillain-Barre Syndrome, Hashimoto's Thyroiditis, Hepatitis, Hughes Syndrome, Idiopathic Pulmonary Fibrosis, Idiopathic Thrombocytopenic Purpura, Irritable Bowel Syndrome, Kawasaki's Disease, Lichen Planus, Lupoid Hepatitis, Lupus/SLE, Lyme Disease, Meniere's Disease, Mixed Connective Tissue Disease, Myositis: Juvenile Myositis (JM), Juvenile dermatomyositis (JDM), and Juvenile Polymyositis (JPM), Osteoporosis, Pars Planitis, Pemphigus Vulgaris, Polyglandular Autoimmune Syndromes, Polymyalgia Rheumatica, Polymyositis, Primary Biliary Cirrhosis, Primary Sclerosis Cholangitis, Psoriasis, Raynaud's Syndrome, Reiter's Syndrome, Rheumatic Fever, Rheumatoid Arthritis, Scleritis, Scleroderma, Sticky Blood Syndrome, Still's Disease, Stiff Man Syndrome, Sydenham's Chorea, Takayasus Arteritis, Temporal Arteritis, Ulcerative Colitis, Uveitis, Vasculitis, Wegener's Granulomatosis and Wilson's Syndrome, preferably the autoimmune disease is vasculitis such as catastrophic anti-phospholipid syndrome (also named Asherson's syndrome), Giant Cell Arteritis and anti-ANCA vasculitis or myasthemia gravis, refractory celiac disease, autoimmune uveitis such as Behcet's Disease, pemphigus vulgaris, giant cell myocarditis, Graves' disease, Addison's disease and granulomatosis with polyangiitis.

Preferably the allergic disease is asthma, atopic allergy or atopic dermatitis.

Preferably the inflammatory or autoinflammatory disease is a chronic inflammatory disease, preferably the chronic inflammatory disease is selected from the group consisting of: inflammatory bowel disease, Chron's disease, ulcerative colitis, celiac disease.

In a preferred embodiment the genetically modified dendritic cell or precursor cell thereof of the invention is for use for the prevention of immune responses against protein replacement therapy, preferably for the treatment of a lysosomal storage disorders or hemophilia.

The present invention also provides a nucleic acid construct comprising:
a nucleic acid sequence a) encoding a chimeric protein consisting of a human invariant chain fused to at least one antigenic peptide or protein or an antigenic fragment thereof, said sequence a) being operatively linked to a first promoter and optionally to a first transcription regulatory sequence and
a nucleic acid sequence b) encoding at least one immuno-modulatory protein, said sequence b) being optionally operatively linked to a second promoter and optionally linked to a second transcription regulatory sequence.

The first promoter and the second promoter may be the same or different as indicated above. The first transcription regulatory sequence and the second transcription regulatory sequence may be the same or different as indicated above.

Preferably the human invariant chain is Iip33, Iip41, Iip35 or Iip43.

The present invention also provides a vector comprising the nucleic acid construct as defined above, preferably said vector is a lentiviral vector, preferably said vector is a mono- or bi-directional vector, preferably the vector is produced using an enveloped viral particle expressing Vpx and/or the vector is produced using a packaging cell wherein said packaging cell is genetically engineered to decrease expression of CD47.

Preferably the vector is an expression vector.

The present invention also provides an in vitro method to produce the genetically modified dendritic cell or a precursor cell thereof as defined above comprising the steps of:
a. Isolating PBMCs from a subject;
b. Isolating CD14⁺ cells from said isolated PBMCs;
c. Incubating said isolated CD14⁺ cells with an effective amount of Vpx;
d. Transducing said isolated CD14⁺ cells with the vector of the invention.

Preferably step d. is performed in the presence of an effective amount of at least one agent, preferably the agent is IL-4 or Granulocyte-macrophage colony-stimulating factor (GM-CSF) or IL-10, preferably the amount of IL-4, of GM-CSF and of IL-10 is between 1 and 1000 ng.

Preferably the PBMCs are isolated from peripheral blood or from leukapheresis.

Still preferably the vector is a lentiviral vector, preferably the amount of said lentiviral vector is between 1 to 100 MOI.

Preferably the effective amount of Vpx is added at day 0 of culture and for about 1 hour to 8 hours, preferably about 6 hours to 8 hours.

The present invention also provides a genetically modified dendritic cell or a precursor cell thereof obtainable by the method as described above.

The present invention also provides an in vitro method to produce IL-10-producing CD49b⁺ LAG-3⁺ Tr1 cells comprising the steps of:
a) isolating PBMCs from a blood sample of a subject;
b) exposing said isolated PBMCs in appropriate culture conditions with an effective amount of a genetically modified dendritic cell or a precursor cell thereof as defined above.

Preferably the ratio PBMC:genetically modified dendritic cell or precursor thereof is between 5:1 and 10:1.

The present invention also provides an IL-10-producing CD49b⁺ LAG-3⁺ Tr1 cell obtainable by the method as defined above, preferably for medical use.

Preferably said IL-10-producing CD49b⁺ LAG-3⁺ Tr1 cells will be infused at different concentration range between 1×10⁴ to 20×10⁷, preferably from 3×10⁵ to 20×10⁶ cells.

The present invention also provides an in vitro method to produce antigen-specific FOXP3⁺ T cells comprising the steps of:
a) isolating PBMCs from a blood sample of a subject;
b) exposing said isolated PBMCs in appropriate culture conditions with an effective amount of a genetically modified dendritic cell or precursor cell thereof as defined above.

Preferably the genetically modified dendritic cell or precursor cell thereof expresses at least indoleamine 2,3-dioxygenase (IDO).

The present invention also provides the antigen-specific FOXP3⁺ T cell obtainable according to the method as described above, preferably for medical use.

Preferably said antigen-specific FOXP3⁺ T cells will be infused at different concentration range between 1×10⁴ to 20×10⁷, preferably between 3×10⁵ to 20×10⁶ cells.

The present invention also provides a pharmaceutical composition comprising the genetically modified cell of the invention or the IL-10-producing CD49b⁺ LAG-3⁺ Tr1 cell as defined above or the antigen-specific FOXP3⁺ T cell as defined above or any combination thereof and a pharmaceutically acceptable carrier.

Preferably the composition further comprises a therapeutic agent.

Said therapeutic agent may be any agent known by the skilled person to treat at least one condition of the invention such as but not limited to an immunosuppressant agent, a steroid, rapamycin, mycophenolate mofetil, rituximab, methotrexate, fludarabine, an anti-inflammatory agent, an anti-allergy agent.

The additional therapeutic agents include, but are not limited to, immunosuppressive agents (e.g., antibodies against other lymphocyte surface markers (e.g., CD40, alpha-4 integrin) or against cytokines), other fusion proteins (e.g., CTLA-4-Ig (Orencia®), TNFR-Ig (Enbrel®)), TNF-a blockers such as Enbrel, Remicade, Cimzia and Humira, cyclophosphamide (CTX) (i.e. Endoxan®, Cytoxan®, Neosar®, Procytox®, Revimmune™), methotrexate (MTX) (i.e. Rheumatrex®, Trexall®), belimumab (i.e. Benlysta®), or other immunosuppressive drugs (e.g., cyclosporin A, FK506-like compounds, rapamycin compounds, or steroids), anti-proliferatives, cytotoxic agents, or other compounds that may assist in immunosuppression.

In some embodiments, the additional therapeutic agent functions to inhibit or reduce T cell activation and cytokine production through a separate pathway. In one such embodiment, the additional therapeutic agent is a CTLA-4 fusion protein, such as CTLA-4 Ig (abatacept). CTLA-4 Ig fusion proteins compete with the co-stimulatory receptor, CD28, on T cells for binding to CD80/CD86 (B7-1/B7-2) on antigen presenting cells, and thus function to inhibit T cell activation. In some embodiments, the additional therapeutic agent is a CTLA-4-Ig fusion protein known as belatacept.

Belatacept contains two amino acid substuitutions (L104E and A29Y) that markedly increase its avidity to CD86 in vivo. In another embodiment, the additional therapeutic agent is Maxy-4.

In another embodiment, the second therapeutic is a second agent that induces IDO expression. Second therapeutics that induce IDO expression are described in Johnson, et al, Immunotherapy, 1(4):645-661 (2009), and U.S. Pat. Nos. 6,395,876 and 6,451,840. In one embodiment, the second therapeutic that induces IDO expression is a nanoparticle loaded with an expression vector that encodes an IDOI or ID02 polypeptide.

In another embodiment, the second therapeutic agent preferentially treats chronic transplant rejection or GvHD, whereby the treatment regimen effectively targets both acute and chronic transplant rejection or GvHD. In another embodiment the second therapeutic is a TNF-α blocker. In another embodiment, the second therapeutic agent increases the amount of adenosine in the serum, see, for example, WO 08/147482. In some embodiments, the second therapeutic is CD73-Ig, recombinant CD73, or another agent (e.g. a cytokine or monoclonal antibody or small molecule) that increases the expression of CD73, see for example WO 04/084933. In another embodiment the second therapeutic agent is Interferon-beta.

In some embodiments, the compositions are used in combination or succession with compounds that increase Treg activity or production.

Exemplary Treg enhancing agents include but are not limited to glucocorticoid fluticasone, salmeteroal, antibodies to IL-12, ΓENγ, and IL-4; vitamin D3, and dexamethasone, and combinations thereof. Antibodies to other proinflammatory molecules can also be used in combination or alternation with the disclosed compositions. For example, antibodies can bind to IL-6, IL-23, IL-22 or IL-21.

As used herein the term “rapamycin compound” includes the neutral tricyclic compound rapamycin, rapamycin derivatives, rapamycin analogs, and other macrolide compounds which are thought to have the same mechanism of action as rapamycin (e.g., inhibition of cytokine function). The language “rapamycin compounds” includes compounds with structural similarity to rapamycin, e.g., compounds with a similar macrocyclic structure, which have been modified to enhance their therapeutic effectiveness. Exemplary Rapamycin compounds are known in the art.

The language “FK506-like compounds” includes FK506, and FK506 derivatives and analogs, e.g., compounds with structural similarity to FK506, e.g., compounds with a similar macrocyclic structure which have been modified to enhance their therapeutic effectiveness. Examples of FK506-like compounds are known in the art. Preferably, the language “rapamycin compound” as used herein does not include FK506-like compounds.

Other suitable therapeutics include, but are not limited to, anti-inflammatory agents. The anti-inflammatory agent can be non-steroidal, steroidal, or a combination thereof. One embodiment provides oral compositions containing about 1% (w/w) to about 5% (w/w), typically about 2.5% (w/w) or an anti-inflammatory agent. Representative examples of non-steroidal anti-inflammatory agents include, without limitation, oxicams, such as piroxicam, isoxicam, tenoxicam, sudoxicam; salicylates, such as aspirin, disalcid, benorylate, trilisate, safapryn, solprin, diflunisal, and fendosal; acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; fenamates, such as mefenamic, meclofenamic, flufenamic, niflumic, and tolfenamic acids; propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; pyrazoles, such as phenylbutazone, oxyphenbutazone, feprazone, azapropazone, and trimethazone. Mixtures of these non-steroidal anti-inflammatory agents may also be employed.

Representative examples of steroidal anti-inflammatory drugs include, without limitation, corticosteroids such as hydrocortisone, hydroxyl-triamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, triamcinolone, and mixtures thereof.

The present invention also provides a genetically modified dendritic cell or a precursor cell thereof modified with a nucleic acid construct, said construct comprising a nucleic acid sequence encoding IL-10, said sequence being operatively linked to a promoter and optionally to a transcription regulatory sequence and/or optionally to a marker, preferably a selectable marker. The present invention also provides a genetically modified dendritic cell or a precursor cell thereof modified with a nucleic acid construct said construct comprising:
a nucleic acid sequence a) encoding a chimeric protein consisting of a human invariant chain fused to at least one antigenic peptide or protein or an antigenic fragment thereof, said sequence a) being operatively linked to a first promoter and optionally to a first transcription regulatory sequence and
a nucleic acid sequence encoding at least one miRNA target sequence being optionally operatively linked to a second promoter and optionally linked to a second transcription regulatory sequence.

The first promoter and the second promoter may be the same or different as indicated above.

The first transcription regulatory sequence and the second transcription regulatory sequence may be the same or different as indicated above.

Preferably the human invariant chain is Iip33, Iip41, Iip35 or Iip43.

Preferably the genetically modified dendritic cell or precursor cell thereof as above defined is for use in organ and/or bone marrow transplant and/or for the prevention and/or treatment of graft versus host disease or for use in the prevention and/or treatment of a condition selected from the group consisting of: autoimmune disease, allergic disease, inflammatory disease, immune response induced by gene therapy.

Still preferably the genetically modified cell is obtained by transduction with a single vector or a mixture of vectors (for instance lentiviral vectors) coding for different fragments of the antigen (according to known antigen libraries).

In a preferred embodiment the genetically modified dendritic cell or precursor cell thereof is used for the prevention of immune responses against autoantigens, preferably for the treatment of autommune and autoinflammatory diseases.

In a preferred embodiment the genetically modified cell is used for the prevention of immune responses after allogeneic transplantation, preferably for the treatment of organ transplantation.

The skilled in the art will also realize that for nucleic acids encoding proteins or peptides, mutations that results in conservative amino acid substitutions may be made in a nucleic acid to provide functionally equivalent variants, or homologs of a protein or peptide. In some aspects the disclosure embraces sequence alterations that result in conservative amino acid substitution of a nucleic acid.

The present invention will be illustrated by means of non-limiting examples in reference to the following figures.

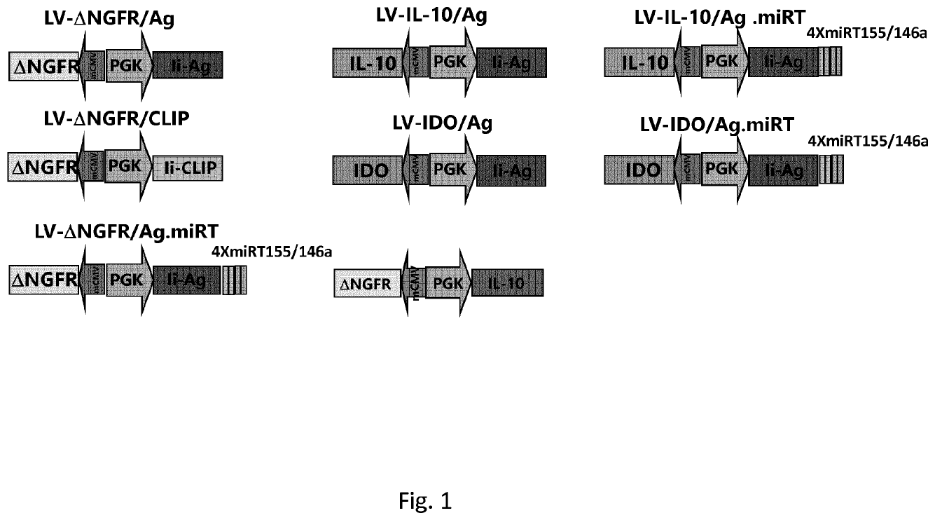

FIG. 1. Lentiviral vector design. Bi-directional LV constructs designed for transduction of DC precursors. CLIP=Class II associated Invariant Chain Peptide; Ii=invariant chain; PGK=phosphoglycerate kinase.

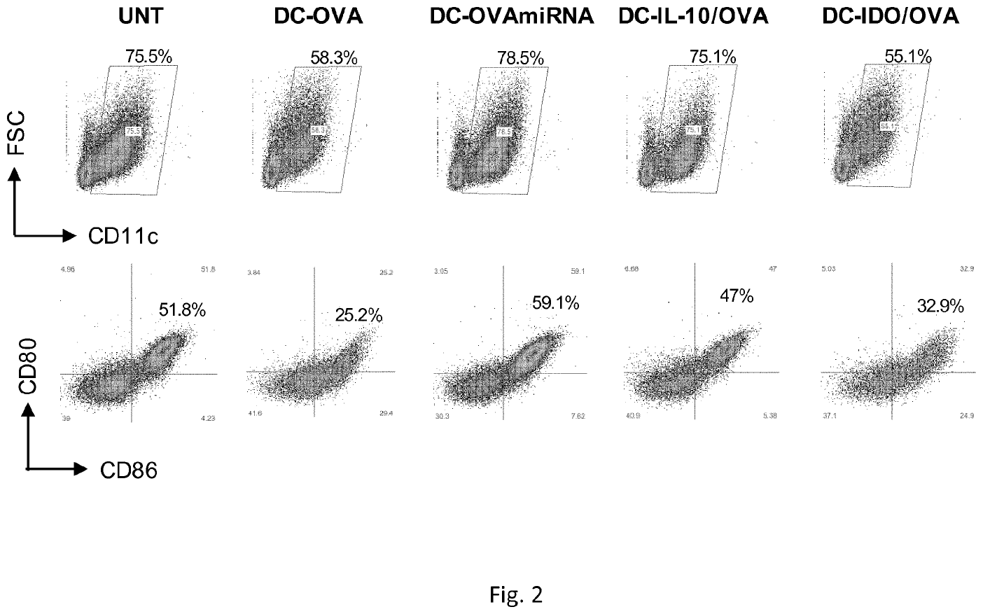

FIG. 2. Generation of LV.DC by LV.IiOVA-mediated gene transfer into bone marrow-derived DC. Bone marrow (BM) cells were differentiated into DC in the presence of GM-CSF and transduced with the indicated LVs on day 2. As control, un-transduced DC (UNT) were used. The expression of CD11c, CD80 and CD86 was analyzed at day 8 of differentiation by FACS. Percentage of positive cells are indicated.

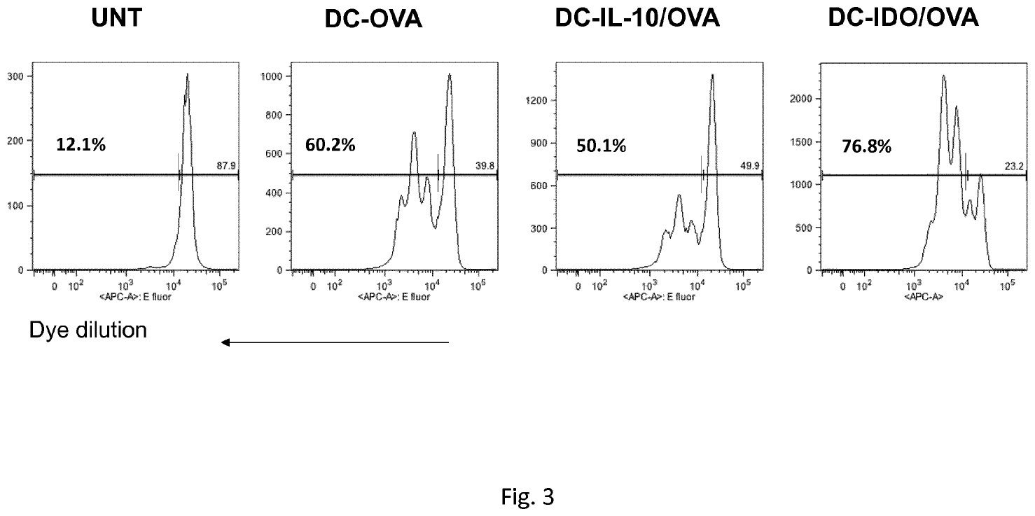

FIG. 3. DC-IL-10/OVA display low stimulatory activity. Bone marrow (BM) cells were differentiated into DC with GM-CSF and transduced with LV-IiOVA, LV-IL-10/OVA, LV-IDO/OVA on day 2. As control, un-transduced DC (UNT) were generated. eFluor-labelled OTII CD4⁺ T cells were stimulated with indicated DC and proliferation was measured by dye dilution after 3 days.

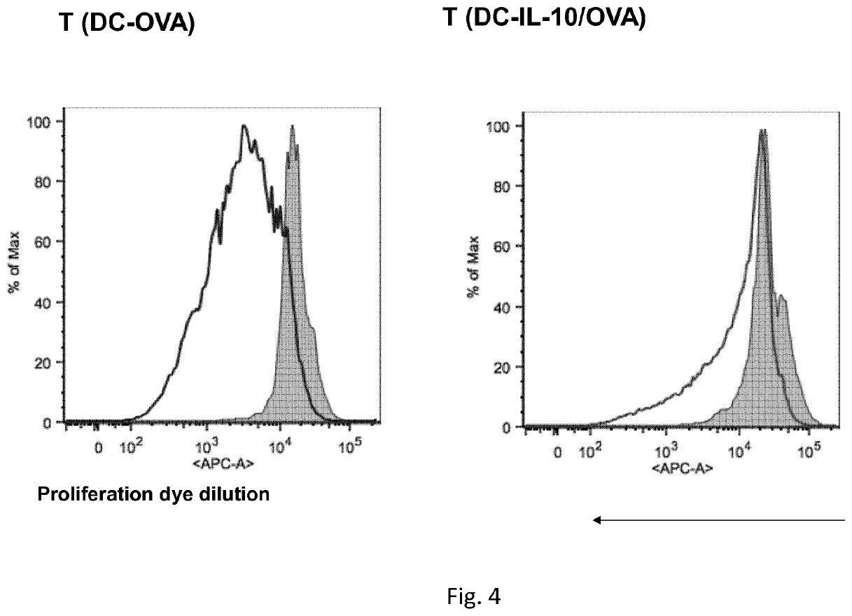

FIG. 4. DC-IL-10/OVA promote antigen-specific hypo-responsiveness. Bone marrow (BM) cells were differentiated into DC with GM-CSF and transduced with LV-IL-10/OVA on day 2. As control, DC-OVA were generated. OTII CD4⁺ T cells were stimulated with LV-DC for 7 days. After culture, cells generated with DC-OVA [T(DC-OVA] and with DC-IL-10/OVA [T(DC-IL-10/OVA] were eFluor-labelled and stimulated with DC-OVA and proliferation was measured by dye dilution after 4 days.

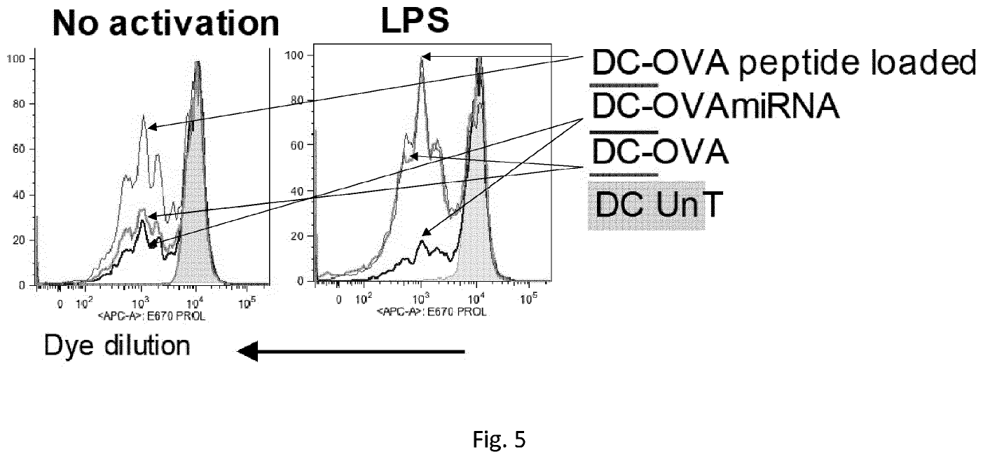

FIG. 5. Activation-dependent up-regulation of miR155 and miR146a limits OVA expression and presentation by DC-OVAmiRNA. Bone marrow (BM) cells were differentiated into DC with GM-CSF and transduced with LV.OVA.miRNA or LV-IiOVA on day 2. DC were left inactivated or activated with LPS for 24 hrs. As control, DC pulsed with OVA peptide and un-transduced DC (DC UNT) were used. eFluor-labeled OTII CD4⁺ T cells were stimulated with the indicated DC, either LPS activated or not. Proliferation was measured by dye dilution after 3 days.

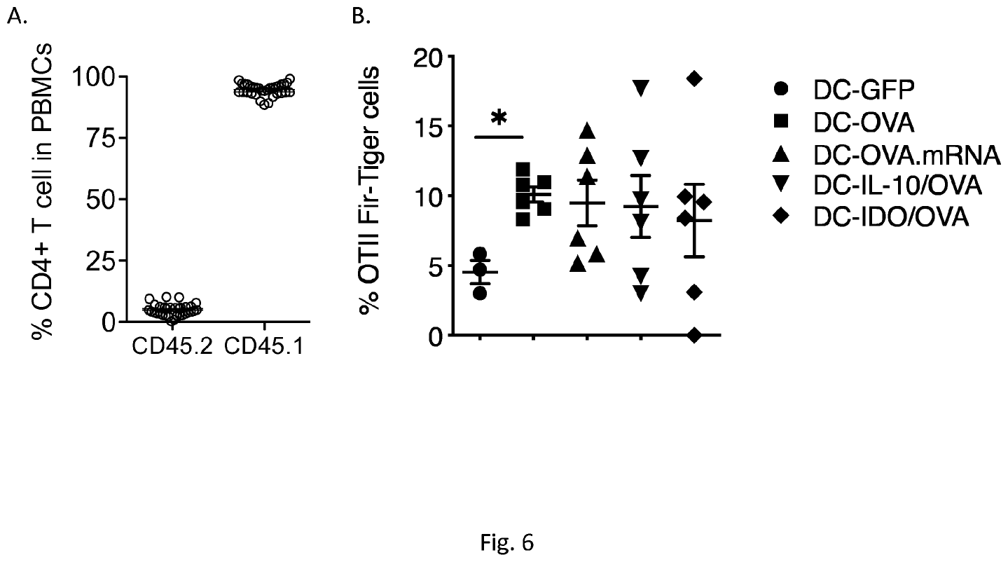

FIG. 6. Administration of LV-DC promotes the expansion of OVA-specific T cells. Chimeric mice obtained by injecting bone marrow cells from CD45.1 (95%) and OT-II/Fir-Tiger (5%) (A) received four injections of DC (DC-IL-10/OVA, DC-IDO/OVA, DC-OVA.miRNA), and DC-OVA or DC-GFP as control. Five weeks after the last DC injection mice were sacrificed and the percentages of CD45.2 OTII firTiger CD4 T cells were determined in the spleen (B) by FACS. *p<0.05 Mann-Whitney U test.

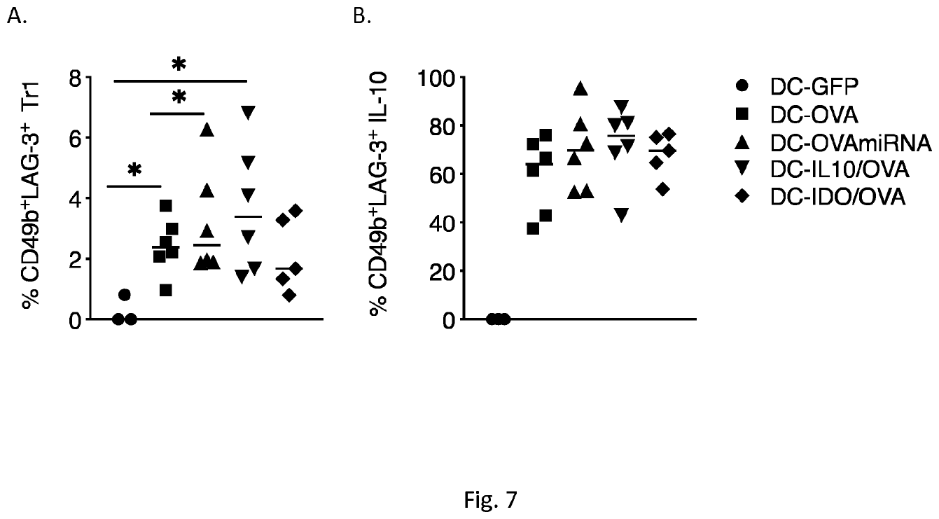

FIG. 7. Induction of IL-10-producing Tr1 cells by DC-IL-10/OVA and DC-OVAmiRNA. Chimeric mice obtained by injecting bone marrow cells from CD45.1 (95%) and OT-II/Fir-Tiger (5%) received four injections of the indicated LV-DCs. Five weeks after the last DC injection, mice were sacrificed and the percentages of CD49b⁺ LAG-3⁺ Tr1 (A), IL10-producing Tr1 cells (GFP⁺) (B) in the spleen were determined by FACS. *<0.05 Mann-Whitney U test.

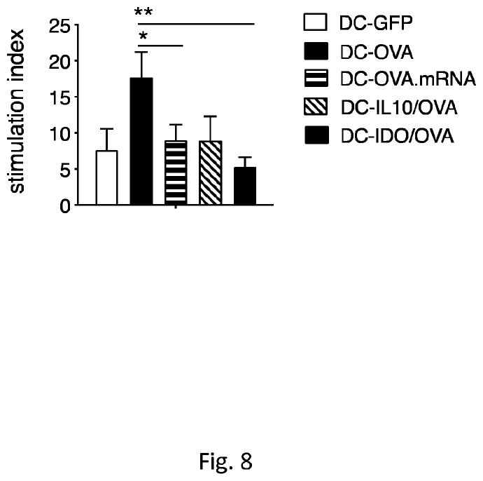

FIG. 8. Hypo-proliferative responsiveness of T cells in vivo activated by LV-DCs. Chimeric mice obtained by injecting bone marrow cells from CD45.1 (95%) and OT-II/Fir-Tiger (5%) received four injections of LV-DCs. Five weeks after the last LV-DC injection, mice were sacrificed and CD4⁺ T cells purified from the spleen were stained with efluor670 and re-stimulated with DC-OVA (T:DC ratio 10:1). Proliferation was measured by dye dilution after 4 days. Data are reported as stimulation index [(% of divided T cell DC-OVA)/(% of divided T cell untreated-DC)]. **<0.005, *<0.05 Mann-Whitney U test.

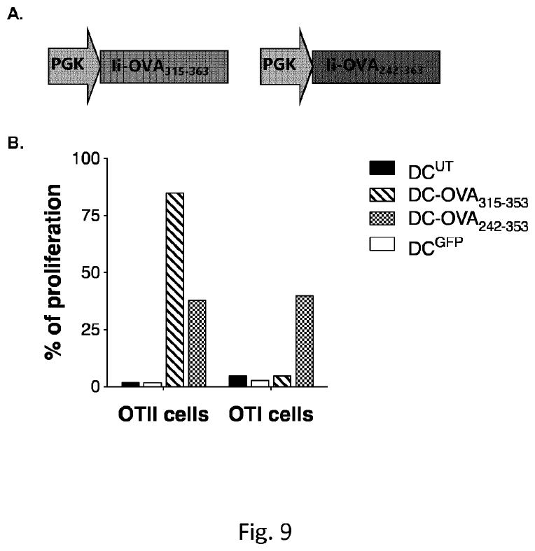

FIG. 9. Generation of LV construct that allow OVA-specific CD4⁺ and CD8⁺ T cell proliferation. Bone marrow (BM) cells were differentiated into DC with GM-CSF and transduced on day 2 with LV encoding for IiOVA₃₁₅₋₃₆₃ containing epitope recognized by OTII CD4⁺ T cells, and for IiOVA₂₄₂₋₃₆₃ containing epitopes recognized by OTII CD4⁺ and OTII CD8⁺ T cells (A). As control DC^{GFP} and un-transduced DC (DC^{UT}). eFluor-labeled OTII CD4⁺ or OTI CD8⁺ T cells were stimulated with the indicated DC. Proliferation was measured by dye dilution after 3 days (B).

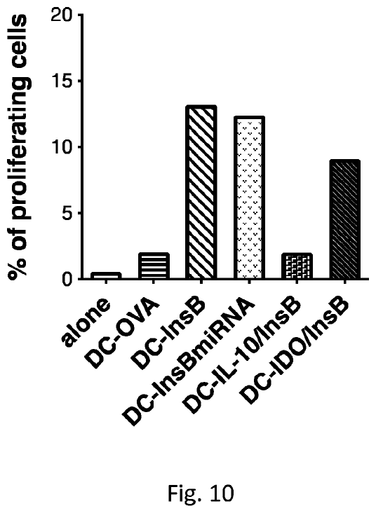

FIG. 10. DC-IL-10/InsB promote hypo-responsiveness in CD4. T cells isolated from diabetic NOD mice. Bone marrow (BM) isolated from NOD mice were differentiated into DC in the presence of GM-CSF and transduced on day 2 with LV-IiInsB₄₋₂₉, LV-IiInsB₄₋₂₉-miRNA, LV-IL-10/InsB₄₋₂₉, and LV-IDO/InsB₄₋₂₉. As control, DC-OVA were generated. eFour-labelled splenic CD4⁺ T cells isolated from diabetic NOD mice were stimulated with the indicated LV-DCs. Proliferation was measured by dye dilution after 3 days of co-culture. % of proliferating cells are depicted.

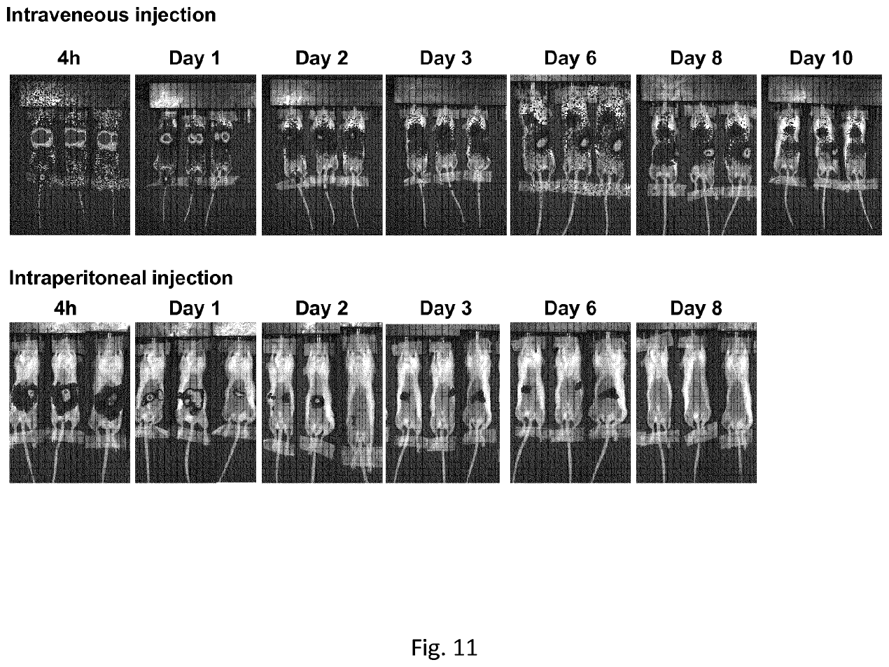

FIG. 11. In vivo localization and life-span of LV-DC. Bone marrow (BM) isolated from Balb/c mice were differentiated into DC in the presence of GM-CSF and transduced on day 2 with LV encoding for luciferase. Balb/c recipient mice were injected with LV-DC (5×10⁶) intravenously or intraperitoneally. Biodistribution and LV-DC survival was monitored by bioluminescence imaging (BLI) at the indicated time points.

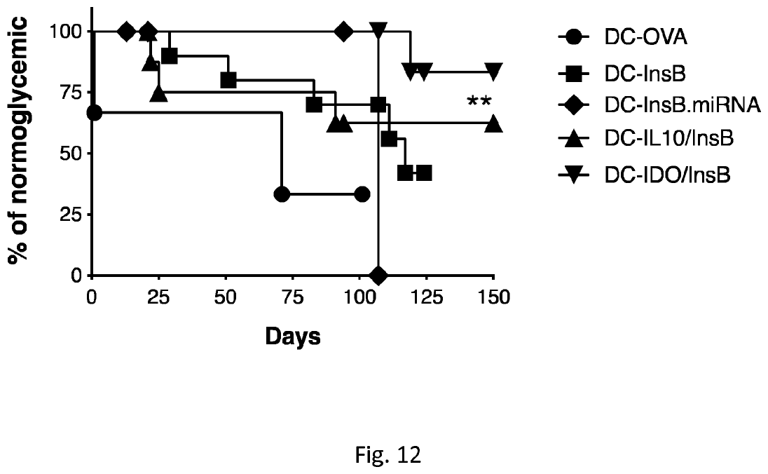

FIG. 12. Autologous LV-DC-cell therapy to protect NOD mice from T1D onset. Bone marrow (BM) isolated from NOD mice were differentiated into DC in the presence of GM-CSF and transduced on day 2 with LV-IiOVA, LV-IiInsB₄₋₂₉, LV-IiInsB₄₋₂₉-miRNA, LV-IL-10/InsB₄₋₂₉, and LV-IDO/InsB₄₋₂₉ to generate DC-OVA, DC-InsB, DC-InsB.miRNA, DC-IL-10/InsB, DC-IDO/InsB. Ten weeks old NOD female mice received three weekly i.v. injections of DC-OVA (n=3), DC-InsB (n=10), DC-InsB.miRNA (n=6), DC-IL-10/InsB (n=9), DC-IDO/InsB (n=11) and blood glucose level was monitored three times a week to evaluate T1 D development. **p<0.005 Log-Rank (Mantel-Cox) test.

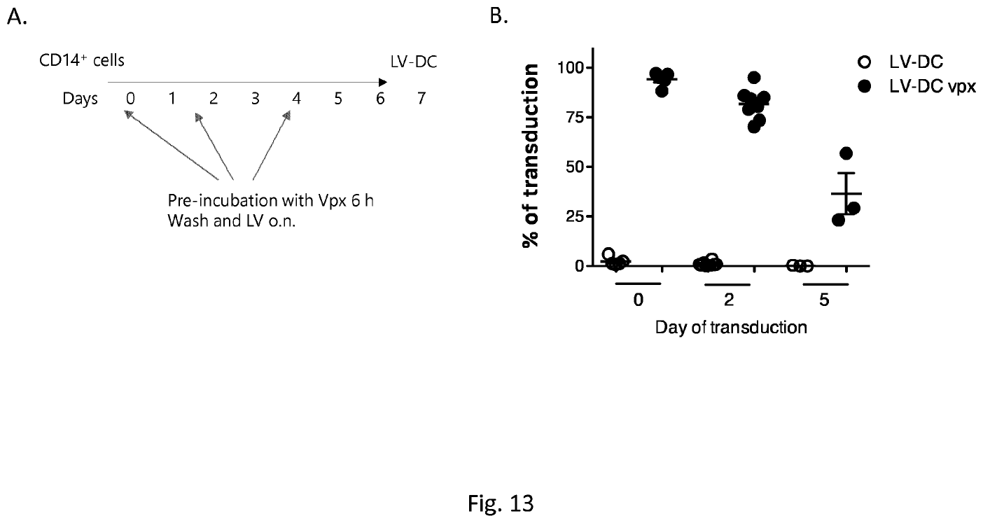

FIG. 13. Development of protocol to efficiently transduce human DC with bidirirectional LV. CD14⁺ cells isolated from peripheral blood of healthy subjects (n=8) were pre-treated with Vpx-VLP for 6-8 hours and then transduced with LV-ΔNGFR/GFP (LV-DC vpx) at day 0, day 2 and day 5 during DC differentiation. As control, DC transduced with LV-ΔNFGR/GFP (LV-DC) were differentiated from the same donors A. Protocol of LV-mediated transduction of monocyte-derived DC. B. Transduction efficiency was quantified based on ΔNGFR expression on differentiated DC.

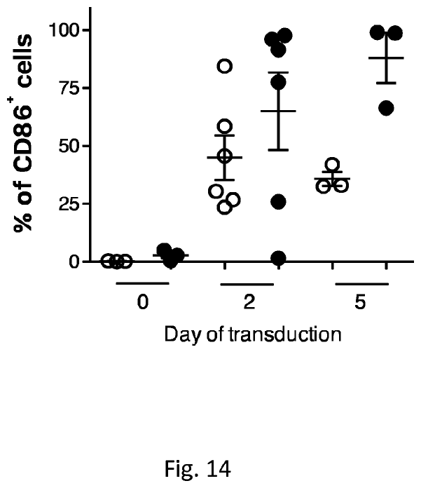

FIG. 14. Pre-treatment with Vpx and LV-mediated transduction do not activate human LV-DC. CD14⁺ cells isolated from peripheral blood of healthy subjects (n=6) were pre-treated with Vpx-VLP for 6-8 hours and then transduced with LV-ΔNGFR/GFP (LV-DC vpx) at day 0, day 2 and day 5 during DC differentiation. As control, DC transduced with LV-ΔNFGR/GFP (LV-DC) were differentiated from the same donors Activation of LV-DC was monitored by expression of CD86. LV-DC transduced in the absence (white symbols) or in the presence of VPX (black symbols).

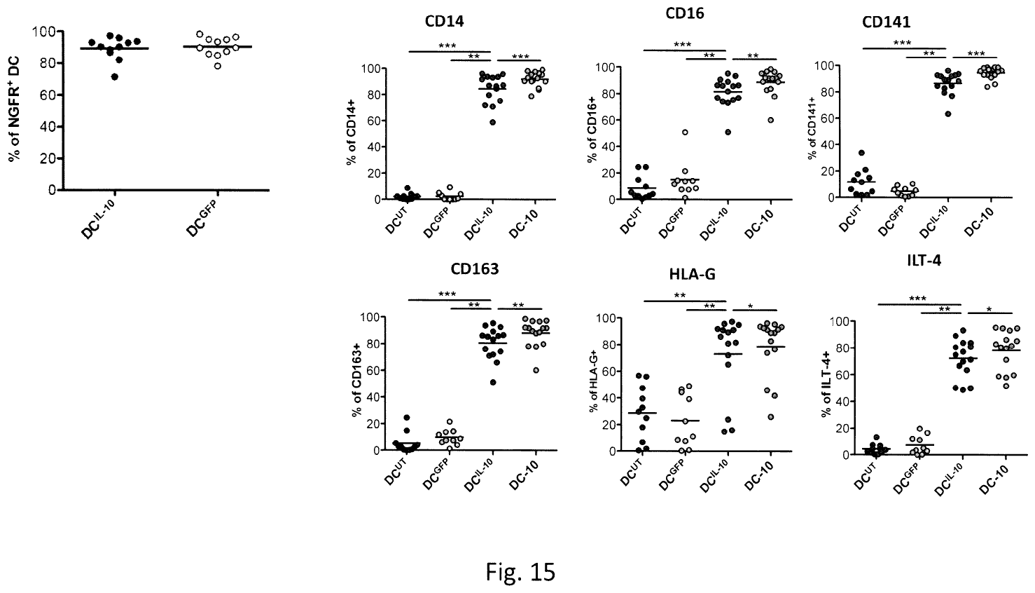

FIG. 15. DC^{IL-10} are phenotipically similar to DC-10. CD14⁺ cells isolated from peripheral blood of healthy subjects (n=11) were treated with Vpx-VLP for 6-8 hours and then transduced with LV-ΔNGFR/GFP (DC^{GFP}) or LV-ΔNGFR/IL-10 (DC^{IL-10}) at day 0 during DC differentiation. As control, DC un-transduced (DC^{UT}) and DC-10 differentiated from the same donors in the presence of GM-SCF/IL-4 and IL-10 were used. A. Transduction efficiency was quantified based on ΔNGFR expression on differentiated DC. B. The expression of the indicated surface markers was assessed by FACS. * P<0.05, **<0.01, Wilcoxon signed rank test.

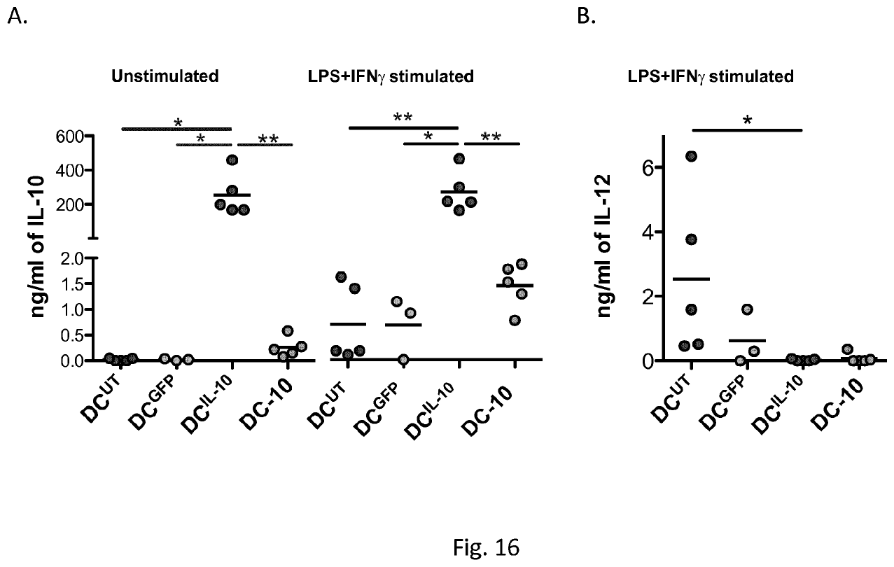

FIG. 16. DC^{IL-10} secreted high levels of IL-10 in the absence of IL-12. CD14⁺ cells isolated from peripheral blood of healthy subjects (n=5) were treated with Vpx-VLP for 6-8 hours and then transduced with LV-ΔNGFR/GFP (DC^{GFP}) or LV-ΔNGFR/IL-10 (DC^{IL-10}) at day 0 during DC differentiation. As control, DC un-transduced (DC^{UT}) and DC-10 differentiated from the same donors in the presence of GM-SCF/IL-4 and IL-10 were used. Resulting cells were left inactivated or activated with LPS/IFNg for 48 hours. Levels of IL-10 (A) and IL-12 (B) were measured in culture supernatants by ELISA (n=5). * P<0.05, **<0.01, Wilcoxon signed rank test.

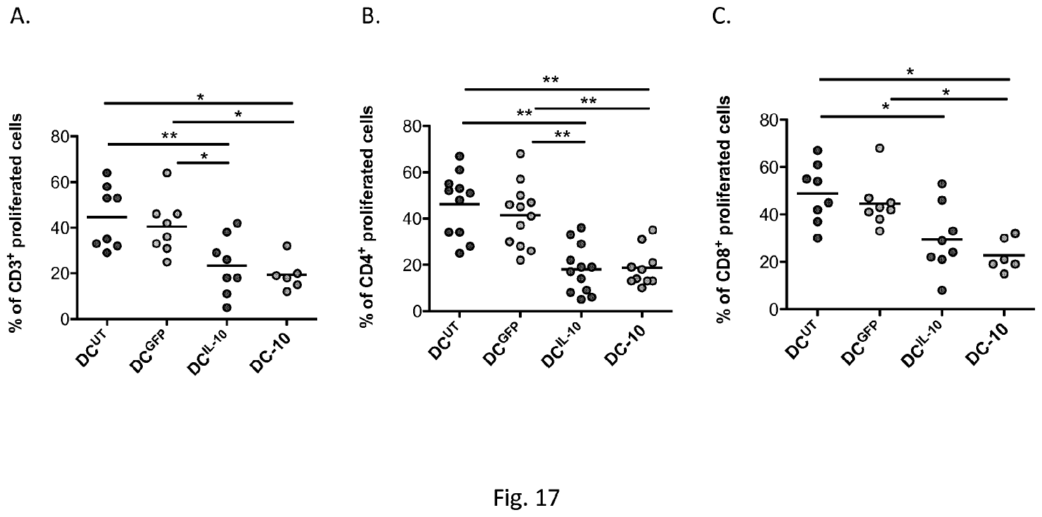

FIG. 17. DC^{IL-10} induce low proliferative response in allogeneic CD3⁺ T cells. CD14⁺ cells isolated from peripheral blood of healthy subjects (n=11) were treated with Vpx-VLP for 6-8 hours and then transduced with LV-ΔNGFR/GFP (DC^{GFP}) or LV-ΔNGFR/IL-10 (DC^{IL-10}) at day 0 during DC differentiation. As control, DC un-transduced (DC^{UT}) and DC-10 differentiated from the same donors in the presence of GM-SCF/IL-4 and IL-10 were used. Allogeneic CD3⁺ T cells were eFlour labelled and stimulated with the indicated DC for 5 days. The percentage of proliferated cells was calculated based on proliferation dye dilution. Proliferation of total CD3⁺ (A), CD3⁺ CD4⁺ (B), and CD3⁺ CD8⁺ T (C) cells are presented. * P<0.05, **<0.01, Wilcoxon signed rank test.

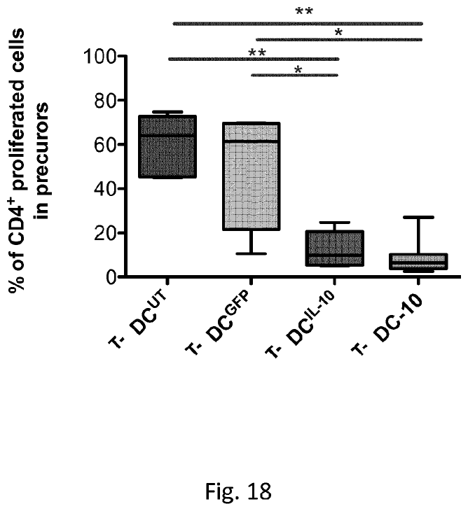

FIG. 18. DC^{IL-10} promote allo-specific anergic CD4⁺ T cells, CD14⁺ cells isolated from peripheral blood of healthy subjects (n=7) were treated with Vpx-VLP for 6-8 hours and then transduced with LV-ΔNGFR/GFP (DC^{GFP}) or LV-ΔNGFR/IL-10 (DC^{IL-10}) at day 0 during DC differentiation. As control, DC un-transduced (DC^{UT}) and DC-10 differentiated from the same donors in the presence of GM-SCF/IL-4 and IL-10 as control. Allogeneic CD4⁺ T cells were stimulated with the indicated DC for 10 days. After 10 days, T cells were eFlour-labelled and re-stimulated with mature DC (mDC) syngeneic to DC used for priming. Percentages of proliferated cells was calculated based on proliferation dye dilution (n=7). * P<0.05, **<0.01, Mann Whitney test.

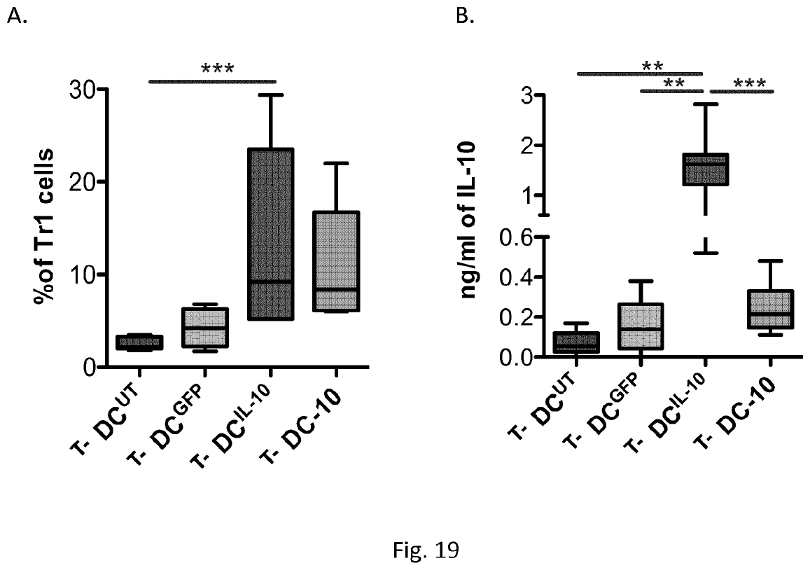

FIG. 19. DC^{IL-10} promote allo-Specific IL-10-producing Tr1 Cells. CD14⁺ cells isolated from peripheral blood of healthy subjects (n=7) were treated with Vpx-VLP for 6-8 hours and then transduced with LV-ΔNGFR/GFP (DC^{GFP}) or LV-ΔNGFR/IL-10 (DC^{IL-10}) at day 0 during DC differentiation. As control, DC un-transduced (DC^{UT}) and DC-10 differentiated from the same donors in the presence of GM-CSF/IL-4 and IL-were used. Allogeneic CD3⁺ T cells were stimulated with the indicated DC for 10 days. A. After 10 days, the percentage of Tr1 cells (CD49b⁺ LAG-3⁺) was evaluated by FACS staining (n=7) B. After 10 days, cells were re-stimulated with mature DC (mDC) syngeneic to DC used for priming and levels of IL-10 were evaluated after 48 hours by ELISA (n=7). * P<0.05, **<0.01, Mann Whitney test.

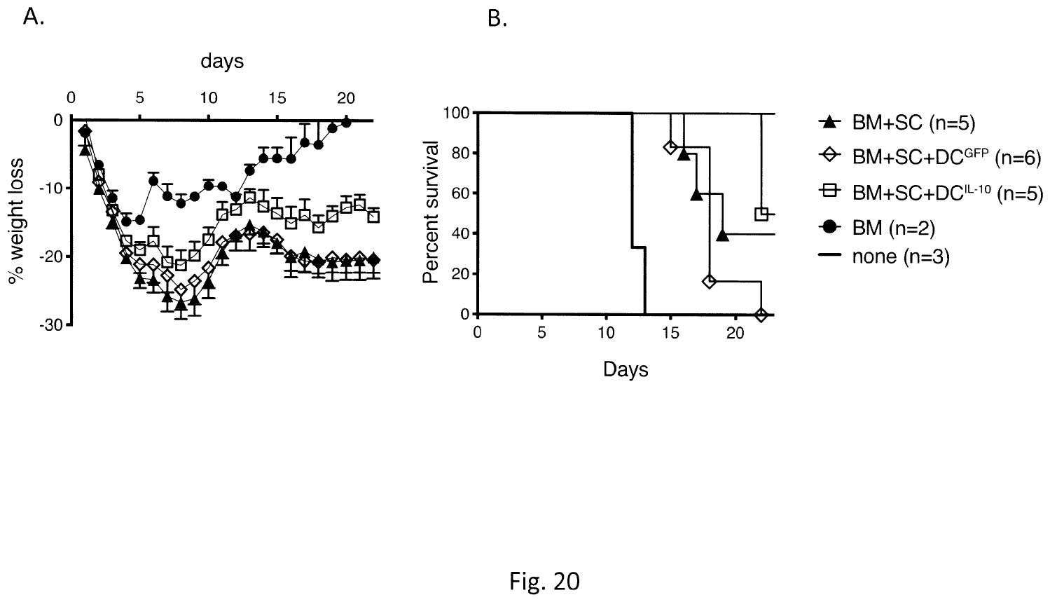

FIG. 20. Adoptive transfer of DC^{IL-10} delays graft-versus host disease. Balb/c bone marrow (BM) cells were differentiated into DC with GM-CSF and transduced on day 2 with LV-ΔNGFR/GFP (DC^{GFP}) or LV-ΔNGFR/IL-10 (DC^{IL-10}). Balb/c mice were lethally irradiated and intravenously injected with C57Bl/6 BM cells (10⁷) and splenocytes (5×10⁶). On day 2 mice were adoptively transferred with DC^{GFP} or DC^{IL-10} (2×10⁶), Wight loos (A) and survival of mice (B) were monitored.

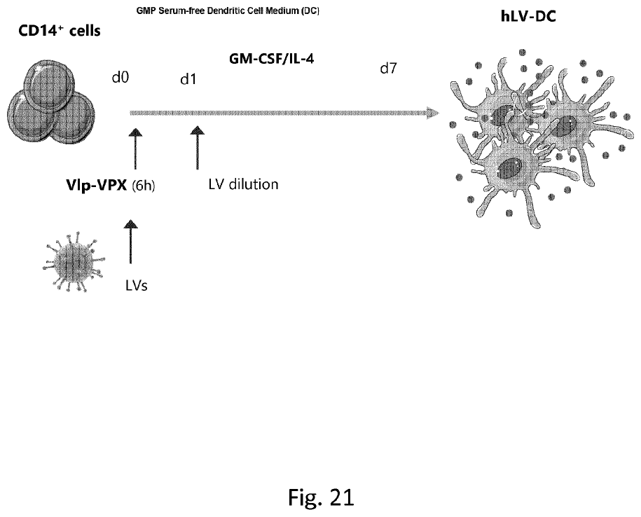

FIG. 21. Protocol to efficiently transduce human DC with bidirectional LVs encoding for a given antigen. CD14⁺ cells isolated from peripheral blood of healthy subjects are cultured in serum free medium and pre-treated with Vpx-VLP (2 ul/well) for 6-8 hours and then transduced with LVs at day 0 during human DC differentiation to obtain human (h)LV-DC. Half of the medium was replaced on day 1 (LV dilution). DC were differentiated in the presence of IL-4 (100 ng/ml) and GM-CSF (100 ng/ml).

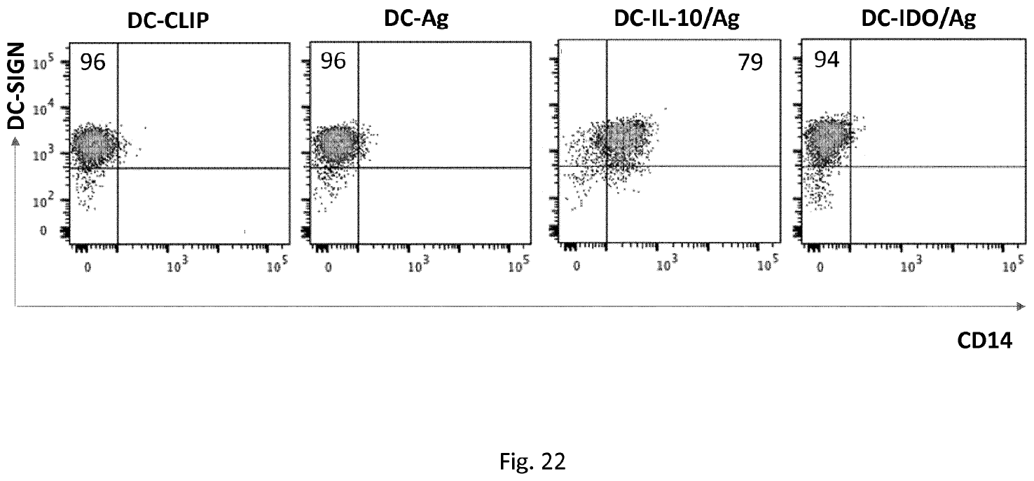

FIG. 22. DC-SIGN expression can be used to monitor LV-DC differentiation in vitro. CD14⁺ cells isolated from peripheral blood of healthy subjects HLA-DQ2.5 or HLA-DQ8 typed were treated with Vpx-VLP for 6-8 hours and then transduced with LV-ΔNGFR/Ag (DC-Ag), LV-IL-10/Ag (DC-IL-10/Ag) or LV-IDO/Ag (DC-IDO/Ag). As control, DC transduced with LV encoding for human CLIP (DC-CLIP) were differentiated in parallel. DC differentiation was monitored by the expression of DC-SIGN and CD14.

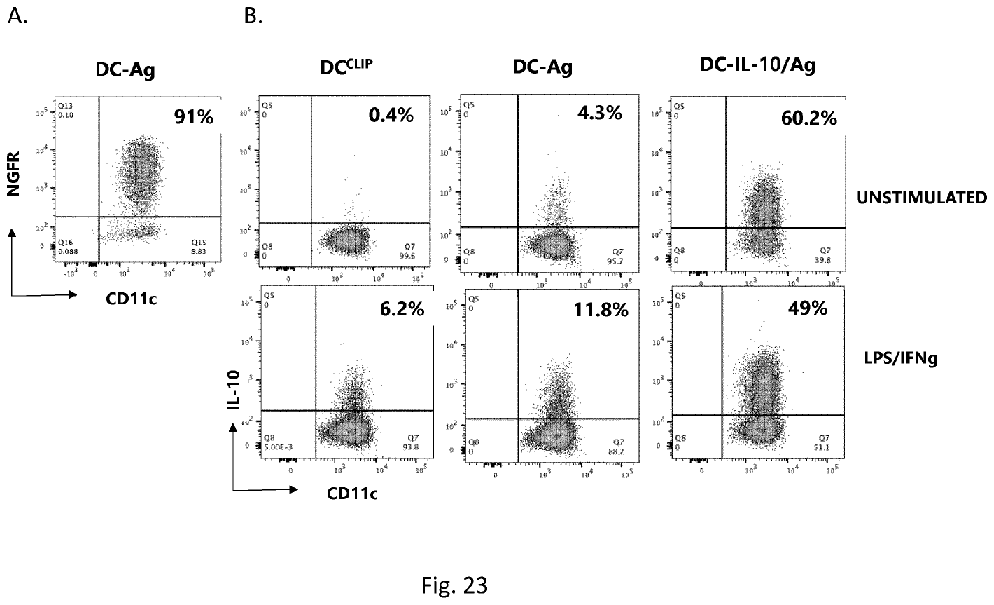

FIG. 23. Transduction efficiency of DC-IL-10/Ag. CD14⁺ cells isolated from peripheral blood of healthy subjects HLA-DQ2.5 or HLA-DQ8 typed were treated with Vpx-VLP for 6-8 hours and then transduced with LV-ΔNGFR/Ag (DC-Ag), LV-IL-10/Ag (DC-IL-10/Ag) at day 0 during DC differentiation. As control, DC transduce with LV-CLIP (DC^{CLIP}) differentiated from the same donors were used. A. Transduction efficiency of DC-Ag was quantified based on ΔNGFR expression. B. To monitor transduction efficiency of DC-IL-10/Ag, DC were left unstimulated or stimulated with LPS (200 ng/ml) and IFN-g (50 ng/ml) for 24 hours. At 6 hours brefeldin was added to cells—Expression of IL-10 was quantified by intracytoplasmic staining. Percentage of positive cells are indicated.

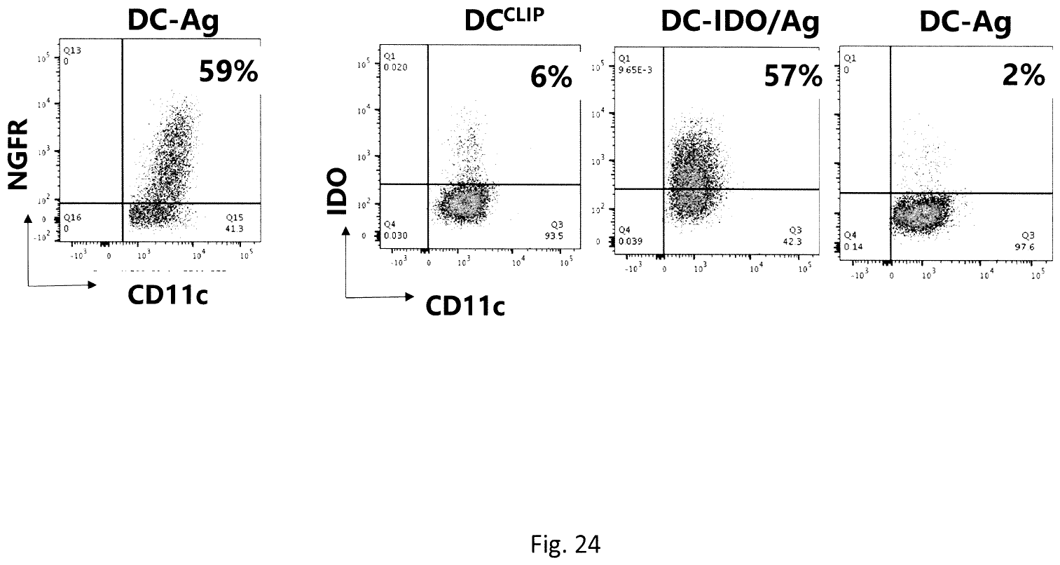

FIG. 24. Transduction efficiency of DC-IDO/Ag. CD14⁺ cells isolated from peripheral blood of healthy subjects HLA-DQ2.5 or HLA-DQ8 typed were treated with Vpx-VLP for 6-8 hours and then transduced with LV-DNFGR/Ag, LV-IDO/Ag (DC-IDO/Ag) at day 0 during DC differentiation. As control, DC transduce with LV-CLIP (DC^{CLIP}) differentiated from the same donors were used. A. Transduction efficiency of DC-Ag was quantified based on ΔNGFR expression. B. Transduction efficiency of DC-IDO/Ag was quantified based on intracytoplasmic IDO expression. Percentage of positive cells are indicated.

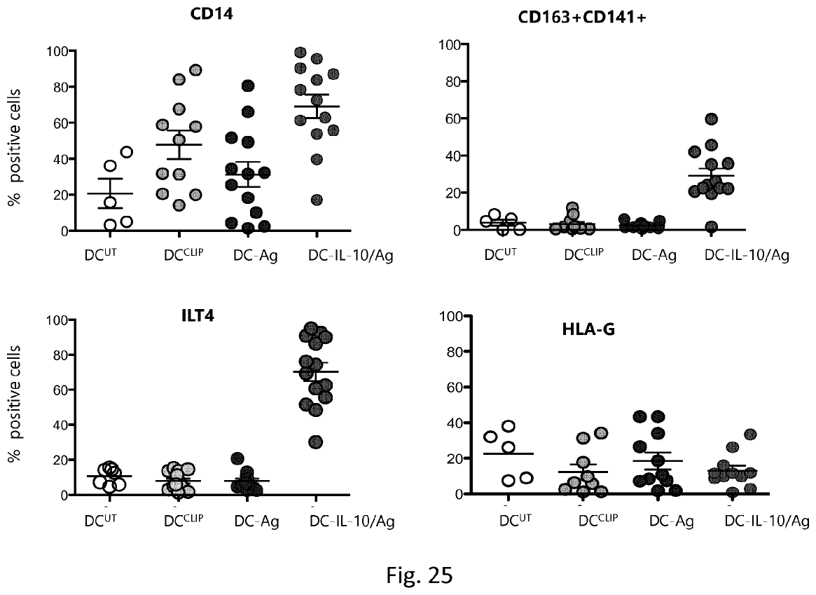

FIG. 25. DC-IL-10/Ag expressed DC-10 associated markers. CD14+ cells isolated from peripheral blood of healthy subjects HLA-DQ2.5 or HLA-DQ8 typed were treated with Vpx-VLP for 6-8 hours and then transduced with LV-ΔNGFR/Ag (DC-Ag), LV-IL-10/Ag (DC-IL-10/Ag) at day 0 during DC differentiation. As control, un-transduced DC (DC^{UT}) of DC transduce with LV-CLIP (DC^{CLIP}) differentiated from the same donors were used. The expression of the indicated surface markers CD14, CD163+CD141+, ILT4 and HLA-G was assessed by FACS.

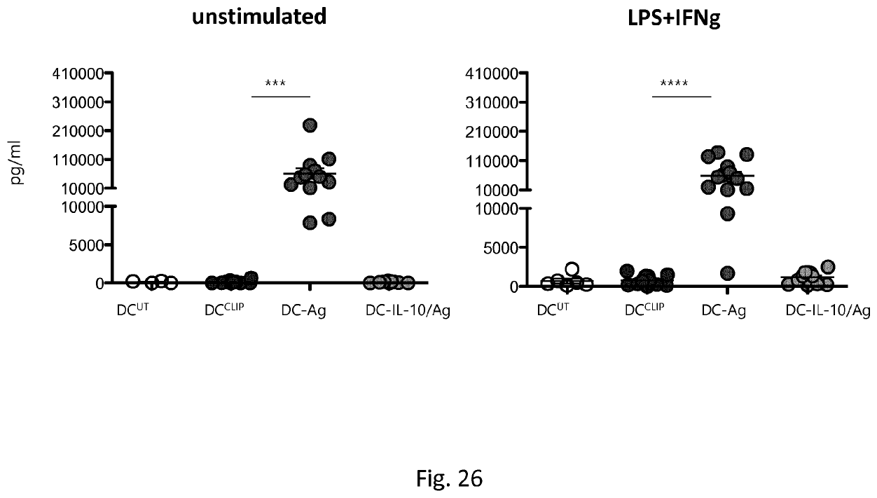

FIG. 26. DC-IL-10/Ag secreted high levels of IL-10 spontaneously and upon activation. CD14⁺ cells isolated from peripheral blood of healthy subjects HLA-DQ2.5 or HLA-DQ8 typed were treated with Vpx-VLP for 6-8 hours and then transduced with LV-ΔNGFR/Ag (DC-Ag), LV-IL-10/Ag (DC-IL-10/Ag) at day 0 during DC differentiation. As control, un-transduced DC (DC^{UT}) of DC transduce with LV-CLIP (DC^{CLIP}) differentiated from the same donors were used. Resulting cells were left inactivated or activated with LPS/IFNγ (200 ng/ml of LPS and 50 ng/ml of IFN-g) for 48 hours. Levels of IL-10 were measured in culture supernatants by ELISA. ***P<0.0001, ****<0.0001, Mann Whitney test.

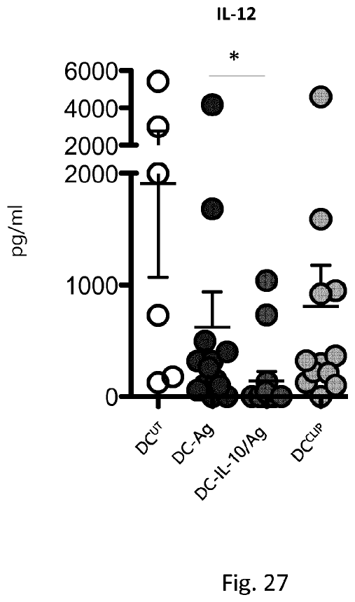

FIG. 27. DC-IL-10/Ag secreted low levels of IL-12 upon activation. CD14⁺ cells isolated from peripheral blood of healthy subjects HLA-DQ2.5 or HLA-DQ8 typed were treated with Vpx-VLP for 6-8 hours and then transduced with LV-ΔNFGR/Ag (DC-Ag), LV-IL-10/Ag (DC-IL-10/Ag) at day 0 during DC differentiation. As control, un-transduced DC (DC^{UT}) of DC transduce with LV-CLIP (DC^{CLIP}) differentiated from the same donors were used. Resulting cells were activated with LPS/IFNγ (200 ng/ml of LPS and 50 ng/ml of IFN-γ) for 48 hours. Levels of IL-12 were measured in culture supernatants by ELISA. * P<0.05 Mann Whitney test.

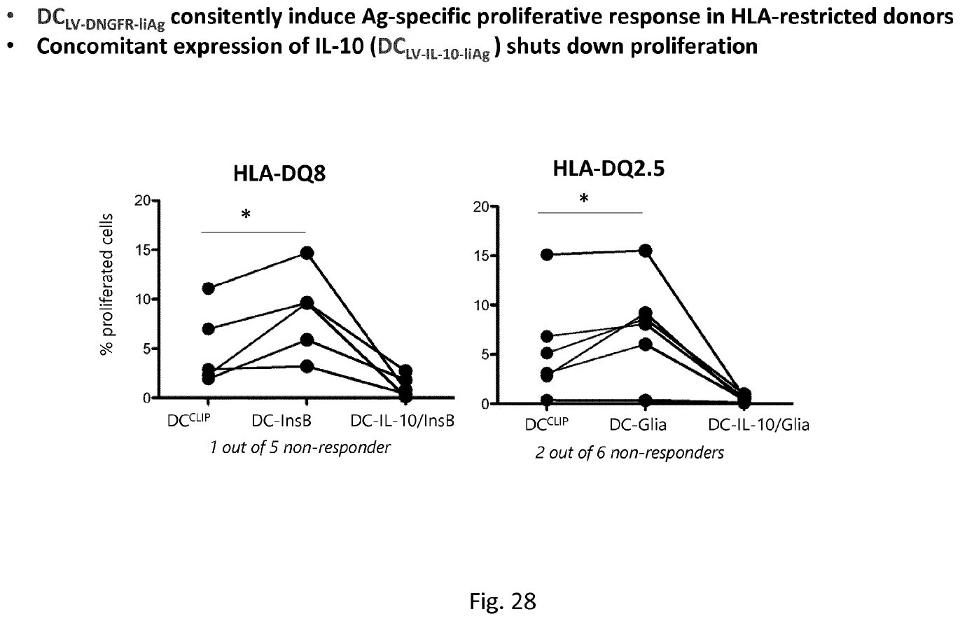

FIG. 28. DC-IL-10/Ag induce low proliferative response in autologous CD3⁺ T cells. CD14⁺ cells isolated from peripheral blood of healthy subjects HLA-DQ2.5 or HLA-DQ8 typed were treated with Vpx-VLP for 6-8 hours and then transduced with LV-ΔNFGR/Ag (DC-Ag), LV-IL-10/Ag (DC-IL-10/Ag) at day 0 during DC differentiation. As control, DC transduce with LV-CLIP (DC^{CLIP}) differentiated from the same donors were used. HLA-DQ8 donors were stimulated with LV-DC encoding for insulin B peptide (InsB, and specifically cells transduced with LV-ΔNFGR/InsB (DC-InsB) or LV-IL-10/InsB (DC-IL-10/InsB) were generated, HLA-DQ2.5 donors were transduced with LV encoding for gliadin peptide (Glia), and specifically cells transduced with LV-ΔNFGR/Glia (DC-Glia) of LV-IL-10/Glia (DC-IL-10/Glia) were generated. Autologous CD3⁺ T cells were eFlour labelled and stimulated with the indicated DC at 10:1 ratio for 6 days. The percentage of proliferated cells was calculated based on proliferation dye dilution. * P<0.05, Wilcoxon signed rank test.

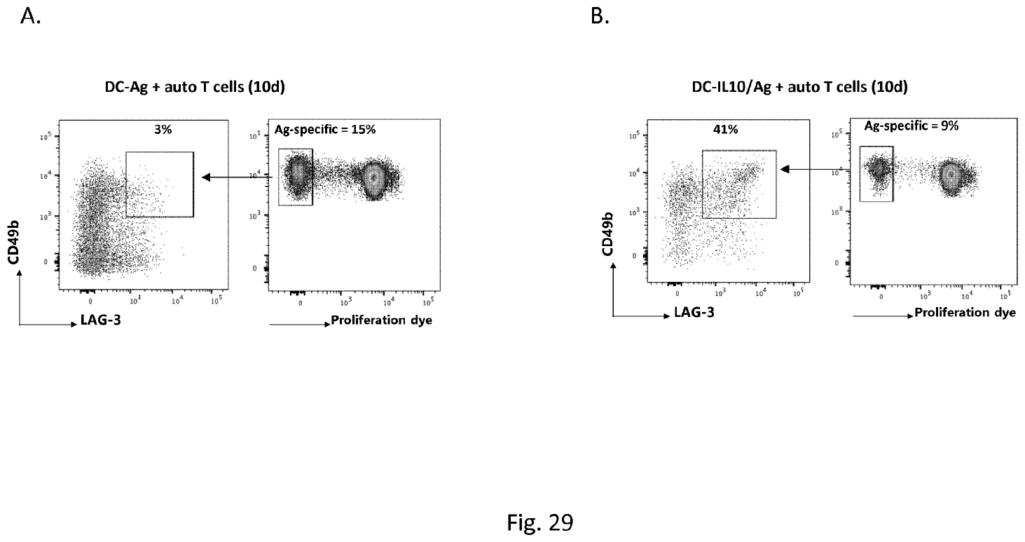

FIG. 29. DC-IL-10/Ag promote Ag-specific Tr1 Cells. CD14⁺ cells isolated from peripheral blood of healthy subjects HLA-DQ2.5 or HLA-DQ8 typed were treated with Vpx-VLP for 6-8 hours and then transduced with LV-ΔNFGR/Ag (DC-Ag) (A), LV-IL-10/Ag (DC-IL-10/Ag) (B) at day 0 during DC differentiation. HLA-DQ8 donors were stimulated with LV-DC encoding for insulin B peptide (InsB, and specifically cells transduced with LV-ΔNFGR/InsB (DC-InsB) or LV-IL-10/InsB (DC-IL-10/InsB) were generated, HLA-DQ2.5 donors were transduced with LV encoding for gliadin peptide (Glia), and specifically cells transduced with LV-ΔNFGR/Glia (DC-Glia) of LV-IL-10/Glia (DC-IL-10/Glia) were generated. Autologous CD3⁺ T cells were eFlour labelled and stimulated the indicated DCs. After 10 days, Tr1 cells (CD49b⁺ LAG-3⁺) with the proliferated cells was evaluated by FACS staining. % of positive cells are presented one out of 4 donors tested.

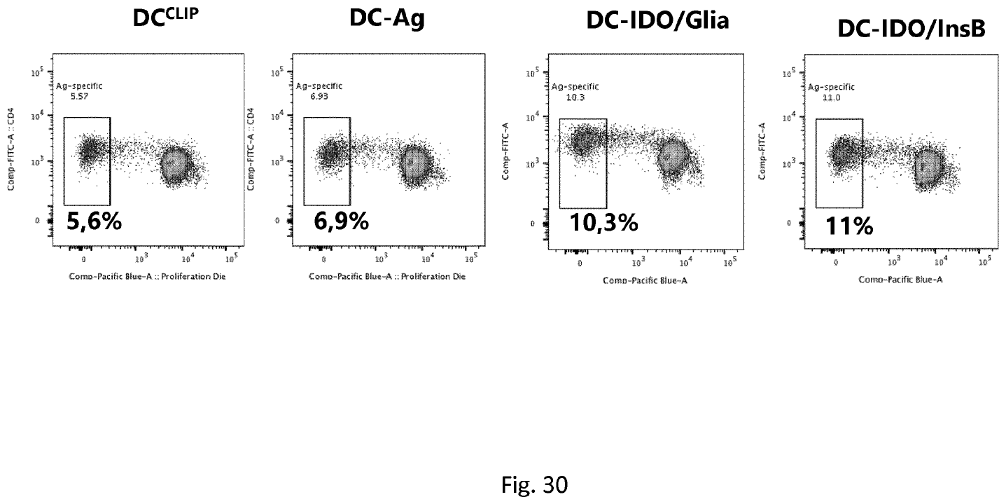

FIG. 30. DC-IDO/Ag induce Ag-specific proliferation in autologous CD3⁺ T cells. CD14⁺ cells isolated from peripheral blood of healthy subjects HLA-DQ2.5 or HLA-DQ8 typed were treated with Vpx-VLP for 6-8 hours and then transduced with LV-ΔNFGR/Ag (DC-Ag), LV-IDO/Ag (DC-IDO/Ag) at day 0 during DC differentiation. As control, DC transduce with LV-CLIP (DC^{CLIP}) differentiated from the same donors were used. HLA-DQ8 donors were stimulated with LV-DC encoding for insulin B peptide (InsB) and specifically cells transduced with LV-ΔNFGR/InsB (DC-InsB) or LV-IL-10/InsB (DC-IL-10/InsB) were generated, HLA-DQ2.5 donors were transduced with LV encoding for gliadin peptide (Glia), and specifically cells transduced with LV-ΔNFGR/Glia (DC-Glia) of LV-IL-10/Glia (DC-IL-10/Glia) were generated. Autologous CD3⁺ T cells were eFlour labelled and stimulated with the indicated DC at 10:1 ratio for 6 days. The percentage of proliferated cells was calculated based on proliferation dye dilution is presented.

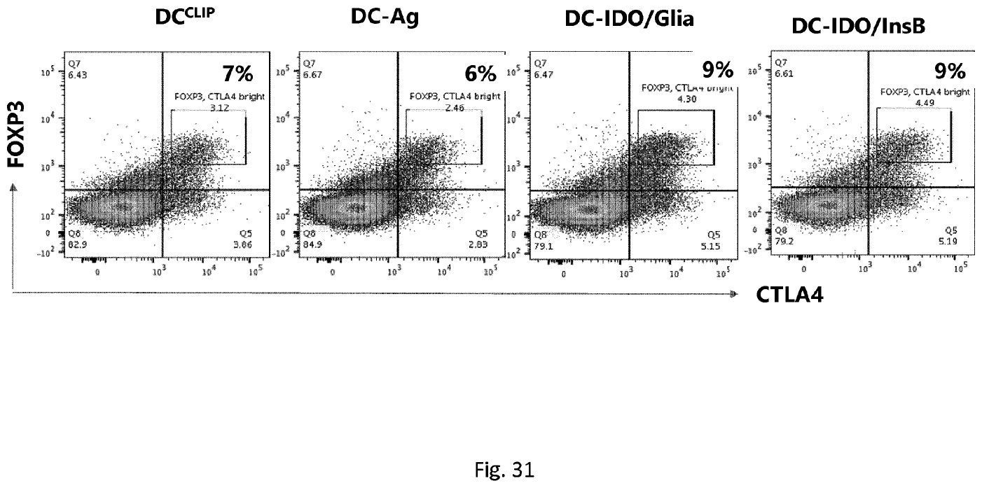

FIG. 31. DC-IDO/Ag promote FOXP3⁺ T cells. CD14⁺ cells isolated from peripheral blood of healthy subjects HLA-DQ2.5 or HLA-DQ8 typed were treated with Vpx-VLP for 6-8 hours and then transduced with LV-ΔNFGR/Ag (DC-Ag), LV-IDO/Ag (DC-IDO/Ag) at day 0 during DC differentiation. HLA-DQ8 donors were stimulated with LV-DC encoding for insulin B peptide (InsB) and specifically cells transduced with LV-IDO/InsB (DC-IDO/InsB) were generated, HLA-DQ2.5 donors were transduced with LV encoding for gliadin peptide (Glia), and specifically cells transduced with LV-IDO/Glia (DC-IDO/Glia) were generated. Autologous CD3⁺ T cells were eFlour labelled and stimulated the indicated DCs. After 10 days, Treg cells (FOXP3+CTLA-4+) was evaluated by FACS staining.

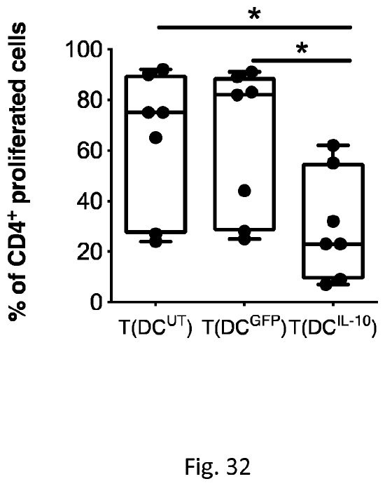

FIG. 32. DC^{IL-10} promote allo-specific Tr1 cells in vitro. CD14⁺ cells isolated from peripheral blood of healthy subjects were pre-treated with Vpx-VLP at day 0, and transduced with LV-ΔNGFR/GFP (DC^{GFP}) or LV-ΔNGFR/IL-10 (DC^{IL-10}) during DC differentiation. In parallel, un-transduced DC were generated (DC^{UT}). On day 7, DC were used to stimulate allogeneic CD4⁺ T cells were isolated from peripheral blood and cultured at 10:1 ratio. After 10 days, T cultured with DC^{UT} [T(DC^{UT})], DC^{GFP}[T(DC^{GFP})] or DC^{IL-10} [T(DC^{IL-10})] were purified using CD4 Miltenyi microbeads, and stained with proliferation dye prior to re-stimulation with LPS-matured DC, differentiated from the same donor as DCs used in primary stimulation. After 3 days, proliferation was evaluated by flow cytometry. Percentage of proliferated cells at the end of the culture was calculated by overall proliferation dye dilution. Each dot represents a single donor, data are shown as mean±STD. *P≤0.05 (Wilcoxon matched pairs test, two-tailed).

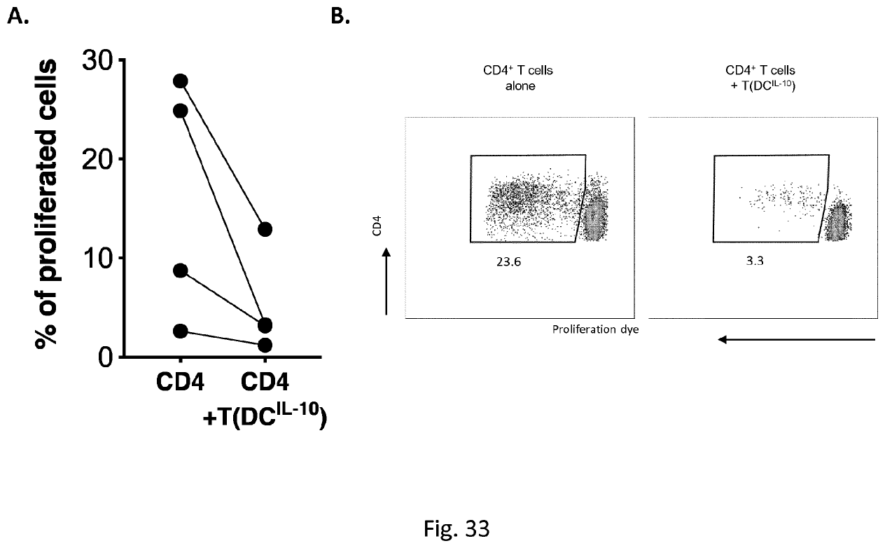

FIG. 33. DC^{IL-10} promote allo-specific Tr1 cells in vitro. CD14⁺ cells isolated from peripheral blood of healthy subjects were pre-treated with Vpx-VLP at day 0, and transduced with LV-ΔNGFR/GFP (DC^{GFP}) or LV-ΔNGFR/IL-10 (DC^{IL-10}) during DC differentiation. In parallel, un-transduced DC were generated (DC^{UT}). On day 7, DC were used to stimulate allogeneic CD4⁺ T cells were isolated from peripheral blood and cultured at 10:1 ratio. After 10 days, T cultured with DC^{UT} [T(DC^{UT})], DC^{GFP} [T(DC^{GFP})] or DC^{IL-10} [T(DC^{IL-10})] were purified using CD4 Miltenyi microbeads and the suppressive activity was evaluated. CD4⁺ T cells autologous to CD4⁺ cells used in primary stimulation were stained with proliferation dye prior to stimulation with mDC, differentiated from the same donor used in primary stimulation, in presence or absence of T(DC^{IL-10}) cells at 1:1 ratio. Percentage of proliferated cells at the end of the culture (left) was calculated by overall proliferation dye dilution. Each dot represents a single donor, data are shown as mean±STD (A). One representative donor is presented (B)

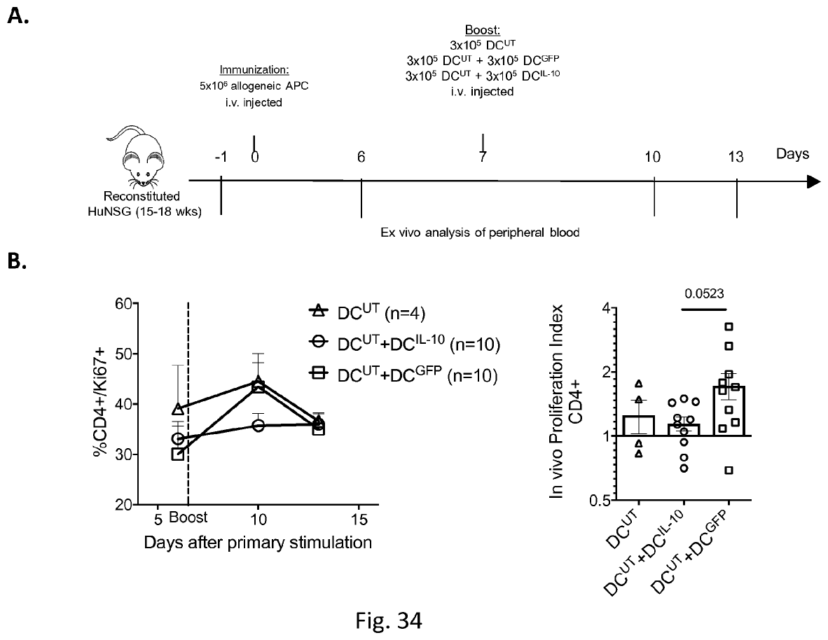

FIG. 34. DC^{IL-10} prevent allo-specific T cell reactivation in huMice. NSG mice were transplanted with 2-4×10⁵ CD34⁺. Reconstituted huMice were immunized with irradiated allogeneic APC by i.v. injection. On day 7, immunized huMice were boosted with autologous DC untransduced (DC^{UT}) alone or with DC^{IL-10} (DC^{UT}+DC^{IL10}) or DC^{GFP} (DC^{UT}+DC^{GFP}); kinetic of PB CD4⁺ cell proliferation is shown.

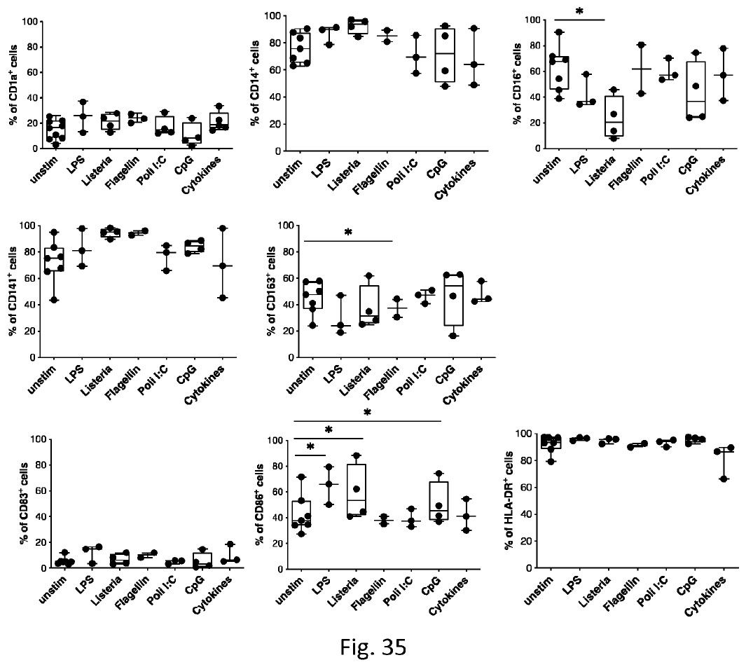

FIG. 35. DC^{IL-10} are phenotypically stable cells. CD14⁺ cells isolated from peripheral blood of healthy subjects were pre-treated with Vpx-VLP at day 0, and transduced with an LV-ΔNGFR/IL-10 (DC^{IL-10}) during DC differentiation. On day 7, DC^{IL-10} were activated with LPS, Heat Killed Listeria monocytogenes, Flagellin S. typhimurium, Poli I:C, ODN2006 (CpG) or a mix of cytokines (IL-1b, TNF-a and IL-6). After 24 hours, the expression of the indicated surface markers CD1a (A), CD141 (B) and CD83 (C) was assessed by FACS. Each dot represents a single donor, data are shown as mean±STD. *P≤0.05 (Wilcoxon matched pairs test, two-tailed).

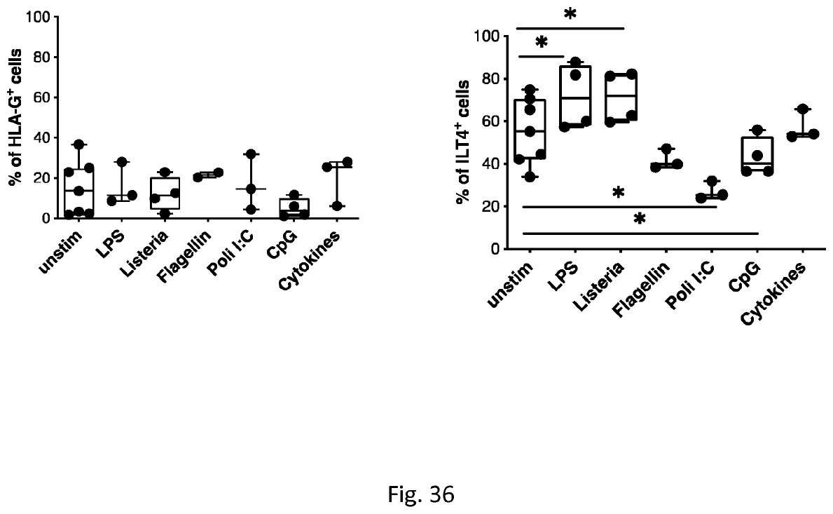

FIG. 36. Activation of DC^{IL-10} modulate ILT4 expression. CD14⁺ cells isolated from peripheral blood of healthy subjects were pre-treated with Vpx-VLP at day 0 and transduced with an LV-ΔNGFR/IL-10 (DC^{IL-10N}) during DC differentiation. On day 7, DC^{IL-10} were activated with LPS, Heat Killed Listeria monocytogenes, Flagellin S. typhimurium, Poli I:C, ODN2006 (CpG) or a mix of cytokines (IL-1b, TNF-α and IL-6). After 24 hours, the expression of the indicated surface markers HLA-G (A) and ILT4 (B) was assessed by FACS. Each dot represents a single donor, data are shown as mean±STD. *P≤0.05 (Wilcoxon matched pairs test, two-tailed).

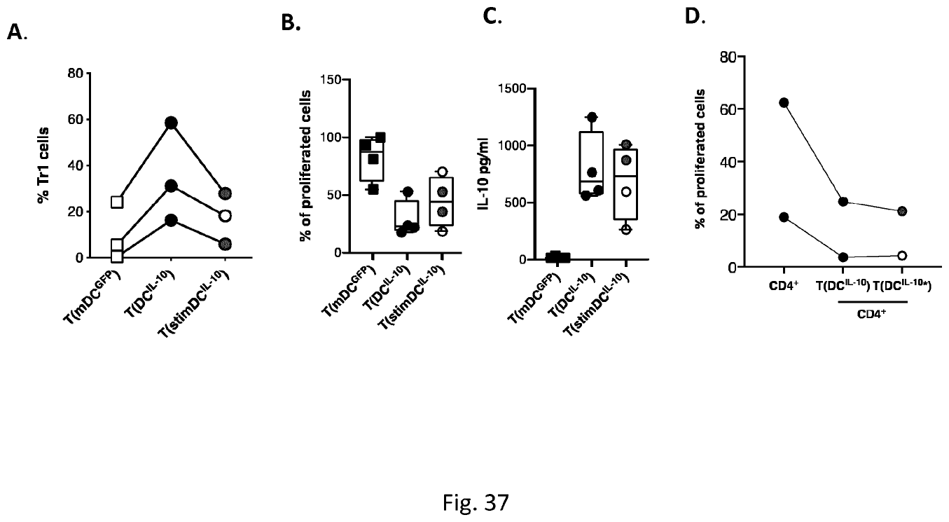

FIG. 37. DC^{IL-10} are functionally stable cells. CD14⁺ cells isolated from peripheral blood of healthy subjects were pre-treated with Vpx-VLP at day 0 and transduced with LV-ΔNGFR/GFP (DC^{GFP}) or LV-ΔNGFR/IL-10 (DC^{IL-10}) during DC differentiation. On day 5 DC^{IL-10} and DC^{GFP} were activate with LPS and LPS or Poli I:C, respectively. On day 7, DC were used to stimulate allogeneic CD4⁺ T cells were isolated from peripheral blood and cultured at 10:1 ratio. After 10 days, T cultured with mDC^{GFP} [T(mDC^{GFP})], DC^{Il-10} [T(DC^{Il-10})] or stimulated DC^{IL-10} [T(stimDC^{IL-10})] were purified using CD4 Miltenyi microbeads. A. Frequency of Tr1 cells. Primed T cells were stained with CD3, CD4, CD45RA, CD49b and LAG-3 to evaluate the percentage of Tr1 cells by flow cytometry. B. T cell proliferation. Primed T cells were stained with proliferation dye prior to re-stimulation with LPS-matured DC, differentiated from the same donor as DC used in primary stimulation. After 3 days, proliferation was evaluated by flow cytometry. Percentage of proliferated cells in the precursor population (right) was calculated with the analysis of peaks, while percentage of proliferated cells at the end of the culture (left) was calculated by overall proliferation dye dilution. C. Cytokine production profile. IL-10 production in cell culture supernatants was evaluated by ELISA D. Suppressive activity. CD4⁺ T cells autologous to CD4⁺ cells used in primary stimulation were stimulated with LPS-matured DC, differentiated from the same donor as DCs used in primary stimulation, in presence or absence of T(DC^{IL-10}) or T(stimDC^{IL-10}) cells at 1:1 ratio. Percentage of proliferated cells at the end of the culture (left) was calculated by overall proliferation dye dilution. Each dot represents a single donor, data are shown as mean±STD. White round symbol indicate cells generated with LPS-activated DC^{IL-10}, red round symbol indicate cells stimulated with Poli I:C-treated DC^{IL-10}.

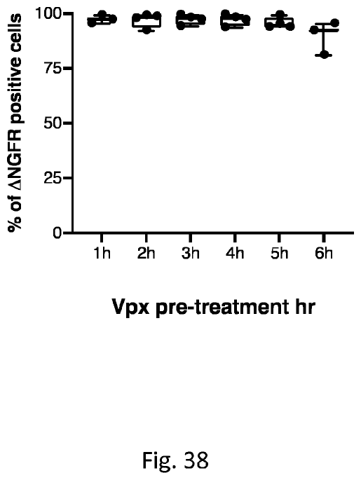

FIG. 38. Vpx time course analysis for efficiently transduction of human DC with bidirectional LVs. CD14⁺ cells isolated from peripheral blood of healthy subjects were pre-treated with Vpx-VLP 5 μl for 1-6 hours and then transduced with LV-ΔNGFR/GFP at day 0 during DC differentiation. Transduction efficiency was quantified based on ΔNGFR expression on differentiated DC.

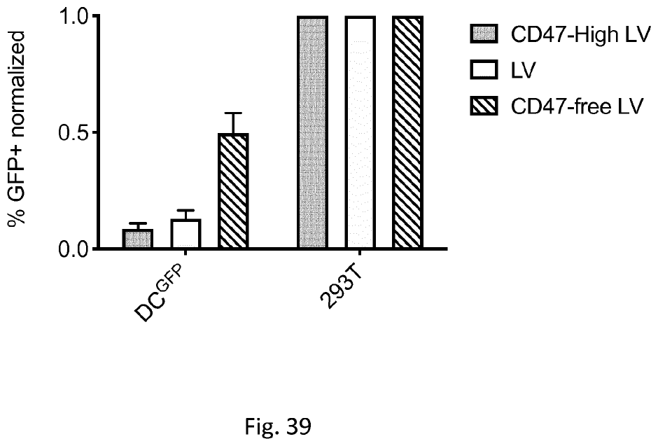

FIG. 39. Increased transgenic expression by humanCD47-free LV particles. CD14⁺ cells isolated from peripheral blood of healthy subjects were transduced with an LV.PGK.GFP at day 0 during DC differentiation (n=5). LV.PGK.GFP were generated using packaging cell lines over-expressing human CD47 (CD47-High LV) or knock-out for CD47 (CD47-free LV), As control, classical LV were used. Human CD47-free LV particles increased transduction efficiency expressed as % of GFP+ cells normalized by % GFP 293T compared to LV particles huCD47-High or wt LV particles carrying normal levels of huCD47.

### DETAILED DESCRIPTION OF THE INVENTION

The practice of the present invention will employ, unless otherwise indicated, conventional techniques of cell biology, molecular biology, histology, immunology, oncology, which are within the capabilities of a person of ordinary skill in the art. Such techniques are explained in the literature.

Immunomodulatory Molecule

An immunomodulatory molecule is an agent (protein or small molecule) that modulates immune responses.

An immune response is a process mediated by cells of the immune system that react against an antigen. The immune response can include immunity to pathogenic microorganisms and its products, or autoimmunity to auto-antigens, allergies against allergenic antigen, and graft rejections against allogeneic antigens. In this process the main cells involved are T cells and B cells, and antigen-presenting cells including macrophages and dendritic cells. Immune responses can be measured by proliferation of T cells, and secretion of cytokine such as IL-2, IL-4, IL-10, and IFNg.

Immunomodulatory molecules include receptors such as PDL-1, PDL-2, ILT-3, ILT-4, HO-1, ICOS-L Gal9, HVME, HLA-G, HLA-E; soluble mediators such as IL-10, IL-35, TGF-a, CTLA-4Ig, PGE2, TNFRs; enzymes such as IDO, Arg1; drugs such as rapamycin, dexamethasone, Vitamin D3, corticosteroids. Preferred immunomodulatory molecule is IL-10 and/or IDO.

As used herein, the term “enhance” may refer to the act of improving, boosting, heightening, or otherwise increasing the presence, or an activity of, a particular target. For example, enhancing an immune response may refer to any act leading to improving, boosting, heightening, or otherwise increasing an immune response. In other examples, enhancing the expression of a nucleic acid may include, but not limited to increase in the transcription of a nucleic acid, increase in mRNA abundance (e.g., increasing mRNA transcription), decrease in degradation of mRNA, increase in mRNA translation, and so forth. In other examples, enhancing the expression of a protein may include, but not be limited to, increase in the transcription of a nucleic acid encoding the protein, increase in the stability of mRNA encoding the protein, increase in translation of the protein, increase in the stability of the protein, and so forth.

MicroRNAs (miRNAs) are small, non-coding RNAs which regulate cellular gene expression by post-transcriptional silencing. When miRNAs are partially complementary to the target mRNA sequences, they typically reduce target mRNA stability and inhibit translation. In contrast, when miRNAs are nearly perfectly complementary to their mRNA targets, they cleave the mRNA, triggering its wholesale destruction. miRNA can achieve tissue specific regulation of systemically delivered and ubiquitously expressed transgenes at post-transcriptional level. miRNAs have distinct expression profiles in different tissues and cell types, which differentially regulate transcriptional profiles of cellular genes and cellular functions, including APCs and immune activation. Therefore, methods provided herein employ immune-related miRNAs (e.g., APC-specific miRNAs) to silence transgene expression in immune cells (e.g., APCs).

miR or miRNA target sequence or “seed sequence” is essential for the binding of the miRNA to the mRNA. The target sequence or seed sequence is a conserved heptametrical sequence which is mostly situated at positions 2-7 from the miRNA 5′-end. Even though base pairing of miRNA and its target mRNA does not match perfect, the “seed sequence” has to be perfectly complementary.

miRNA target sequence is a sequence that modulate the expression of mRNA and consequently of a protein.

miR-15a, miR-16-1, miR-17, miR-18a, miR-19a, miR-20a, miR-19b-l, miR-21, miR-29a, miR-29b, miR-29c, miR-30b, miR-31, miR-34a, miR-92a-l, miR-106a, miR-125a, miR-125b, miR-126, miR-142-3p, miR-146a, miR-150, miR-155, miR-181a, miR-223 and miR-424. More preferably miR155, miR146a, repeated 2 times each.

“Recipient antigen” refers to an antigen expressed by the recipient. As used herein, an “effector cell” refers to a cell, which mediates an immune response against an antigen. An example of an effector cell includes but is not limited to a T cell and a B cell.

As used herein, the term “immune response” includes T cell mediated. and/or B-cell mediated immune responses. Exemplary immune responses include T cell responses, e.g., cytokine production and cellular cytotoxicity, and B cell responses, e.g., antibody production. In addition, the term immune response includes immune responses that are indirectly affected by T cell activation, e.g., antibody production (humoral responses) and activation of cytokine responsive cells, e.g., macrophages. Immune cells involved in the immune response include lymphocytes, such as B cells and T cells (CD4+. CD8+, Th1 and Th2 cells): antigen presenting cells (e.g., professional antigen presenting cells such as dendritic cells, macrophages, B lymphocytes, Langerhans cells, and non-professional antigen presenting cells such as keratinocytes, endothelial cells, astrocytes, fibroblasts, oligodendrocytes); natural killer cells; myeloid cells, such as macrophages, eosinophils, mast cells, basophils, and granulocytes.

An antigen is any substance that causes the immune system to react e.g. by generating T-cells recognizing peptides derived from protein substances, and B-cells producing antibodies against the substance. The antigen will bear one or more epitopes.

Antigen-derived peptide or antigenic peptide or protein is a peptide or protein derived from an antigen processed and presented in the contest of MHC class I or MHC class II molecules to T cells. It is generally composed of between 9 to 12 amino acids. It contains at least one immunodominant peptide or epitope. Antigen-derived peptide fragment or antigenic peptide or antigenic protein fragment is a fragment that is shorter than the antigenic peptide or protein and has the antigenic properties of the peptide or protein.

The term immunodominant peptide (or “epitope”) as used herein is a portion of an antigen that can elicit an immune response, including B and/or T cell responses. An antigen can have one or more immunodominant peptides. Most antigens have many epitopes; i.e., they are multivalent. In some examples, an epitope is roughly about 10 amino acids in size. Preferably, the immunodominant peptide or epitope is about 4-18 amino acids, more preferably about 5-16 amino acids, and even more most preferably 6-14 amino acids, more preferably about 7-12, and most preferably about 8-10 amino acids. One skilled in the art understands that in some circumstances, the three-dimensional structure, rather than the specific linear sequence of the molecule, is the main criterion of antigenic specificity and therefore distinguishes one immunodominant peptide or epitope from another.

In the present invention, in order to allow correct processing and presentation of the immunodominant peptide, the construct comprises a nucleotide sequence coding for the immunodominant peptide and variable flanking regions, each of said flanking regions consisting of 5 to 10 amino acids. For instance, for diabetes, the immune dominant peptide is insulin B₉₋₂₃, while the construct includes a nucleotide sequence encoding insulin B₄₋₂₉.

In the present invention the antigenic peptide or protein or antigenic fragment thereof is from a polypeptide associated with an abnormal physiological response. Such an abnormal physiological response includes but is not limited to autoimmune diseases, allergic reaction, and other diseases of the invention.

Modified Antigen-Derived Peptide or Antigenic Peptide

In the present invention, the antigen-derived peptide (or antigenic peptide) or the immunodominant peptide or epitope may be modified for instance to enhance T cell recognition. Such modification includes but is not limited to: citrullination, deamidation, methylation, carbamylation, glycosylation acylation, acetylation, formylation, amidation, hydroxylation.

For instance, antigen-derived peptides or the immunodominant peptides or epitopes for rheumatoid arthritis are advantageously modified as citrullinated peptides or glycosylated.

Antigen-derived peptides or the immunodominant peptides or epitopes for celiac disease (gliadin) are advantageously modified as deamidated peptides.

The term “antigen” or “Ag” as used herein is defined as a molecule that provokes an immune response. This immune response may involve either antibody production, or the activation of specific immunologically-competent cells, or both. The skilled artisan will understand that any macromolecule, including virtually all proteins or peptides, can serve as an antigen. Furthermore, antigens can be derived from recombinant or genomic DNA. A skilled artisan will understand that any DNA, which comprises a nucleotide sequences or a partial nucleotide sequence encoding a protein that elicits an immune response therefore encodes an “antigen” as that term is used herein. Furthermore, one skilled in the art will understand that an antigen need not be encoded solely by a full-length nucleotide sequence of a gene. It is readily apparent that the present invention includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid. “An antigen presenting cell” (APC) is a cell that is capable of activating T cells, and includes, but is not limited to, monocytes/macrophages, B cells and dendritic cells (DCs).

Invariant Chain

The invariant chain (Ii; CD74) has multiple functions but is best characterized as the main MHC class II (MHCII) chaperone. It is a type II protein consisting of a short cytoplasmic tail, a transmembrane region and a luminal domain that can be further partitioned into a membrane-proximal disordered region, the main MHCII-interacting sequence (CLIP), and a C-terminal trimerization domain (44, 45) (Mice express two Ii isoforms, p31 and p41, the latter resulting from alternative splicing (46). In humans, the corresponding isoforms are known as p33 and p41. Additionally, around 20% of the Ii mRNAs are translated from an upstream start codon that generates the p35 and p43 isoforms. These bear a 16-amino acid cytoplasmic extension including a strong di-arginine (RxR) ER retention motif (47-49).

Synthesized alongside MHCIIs, Ii can be viewed as: (i) a GUARDIAN that controls access to the MHCII groove; (ii) a SCAFFOLD that assists folding and pairing of α and β MHCII chains; and (iii) a LEADER that directs MHCIIs to the endosomal pathway. It is well established that these Ii functions depend primarily on the ability of its CLIP region to occupy the peptide groove of MHCIIs. Numerous reports showed that Ii proteolysis in endosomes allows HLA-DM to free the groove of CLIP and to catalyze the binding of nominal antigenic peptides (reviewed (50)).

The invariant chain of the MHC II molecule (Ii, invariant chain, MHC II gamma chain) is the sequence described most often in the literature as being able to mediate targeting. Various variants of the invariant chain in humans are described and are also referred to as IiP33, IiP41, IiP35 and IiP43 (51) and which are suitable as targeting modules. Further sequences suitable as targeting module for the purposes of the invention are the beta chain of the MHC II molecule (52). Fragments of said sequences are also suitable as targeting module.

Invariant chain is a protein that in humans is encoded by the CD74 gene. It is a polypeptide involved in the formation and transport of MHC class II protein. The nascent MHC class II protein in ER binds a segment of the invariant chain (CLIP) in order to shape the peptide binding groove and prevent formation of a closed conformation. The invariant chain facilitates MHC class II export from the ER in a vesicle endosome containing the endocytosed antigen proteins (from the exogenous pathway).

Here the term invariant chain covers all naturally occurring or artificially generated full length or fragmented homologous gene and proteins of a certain similarity to human invariant chain.

Vpx

Myeloid cells, such as dendritic cells and macrophages are relatively refractory to vector transduction, in particular lentiviral vector transduction, as a result of the myeloid-specific restriction factor, SAMHD1. SIVmac/HIV-2 and related viruses relieve the SAMHD1-mediated restriction by encoding Vpx, a virion-packaged accessory protein that induces the degradation of SAMHD1 upon infection. HIV-1 does not encode Vpx and cannot package the protein. Suitably, the Vpx packaging motif may be packaged in the lentiviral vector virions, for instance may be placed in the p6 region of the Gag/Pol expression vector that is used to generate the lentiviral vector virions which in turn package Vpx in high copy number. Alternatively, Vpx may be provided to DC or precursor cells thereof by pretreatment of the cells with virus-like particles (VLP) that contain Vpx

Marker

In the present invention, a marker is preferably a selectable marker such as ΔNGFR as described herein and whose coding sequence is included the nucleic acid construct in order to allow selection of transduced cells. An alternative can be the truncated form of CD19 in which the deletion of the cytoplasmic domain of CD19 abolishes the signaling pathway [93].

Bicistronic Constructs

Bicistronic vectors or constructs are constructs in which two factors are expressed either using multiple promoters or including internal ribosome entry site (IRES) elements. IRES elements are nucleotide sequences that allow for translation initiation in the middle of a messenger RNA (mRNA) sequence.

Vector

In addition to the major elements identified above for the vector, the vector also includes conventional control elements necessary which are operably linked to the nucleic acid sequence in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized. As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the nucleic acid sequences.

Another vector element that may be used is an internal ribosome entry site (IRES). An IRES sequence is used to produce more than one polypeptide from a single gene transcript. An IRES sequence would be used to produce a protein that contain more than one polypeptide chains.

Selection of these and other common vector elements are conventional and many such sequences are available.

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1 a promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268: 1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

In another embodiment, the native promoter for the nucleic acid sequence will be used. The native promoter may be used when it is desired that expression of the nucleic acid should mimic the native expression. The native promoter may be used when expression of the nucleic acid must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3: 1002-9 (1996); alpha-fetoprotein (AFP) promoter, Arbuthnot et al., Hum. Gene Ther., 7: 1503-14 (1996)), bone osteocalcin promoter (Stein et al., Mol. Biol. Rep., 24: 185-96 (1997)); bone sialoprotein promoter (Chen et al., J. Bone Miner. Res., 11:654-64 (1996)), CD2 promoter (Hansal et al., J. Immunol., 161: 1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan. In some embodiments, the promoter is the muscle specific promoter Desmin460 or the truncated muscle creatine kinase (tMCK) promoter.

The skilled artisan will also realize that in the case of nucleic acid encoding proteins or polypeptides, that mutations that results in conservative amino acid substitutions may be made in a nucleic acid sequence to provide functionally equivalent variants, or homologs of a protein or polypeptide. In some aspects the disclosure embraces sequence alterations that result in conservative amino acid substitution of a nucleic acid sequence.

Dendritic Cell

A dendritic cell is a professional antigen-presenting cell of the immune system with the ability to process and present antigen to T cells.

The term “dendritic cell” or “DC” refers to any member of a diverse population of morphologically similar cell types found in lymphoid or non-lymphoid tissues. These cells are characterized by their distinctive morphology, high levels of surface MHC-class II expression. DC can be isolated from a number of tissue sources. DC have a high capacity for sensitizing MHC-restricted T cells and are very effective at presenting antigens to T cells in situ.

The antigens may be self-antigens that are expressed during T cell development and tolerance, and foreign antigens that are present during normal immune processes.

Precursor Cell of a Dendritic Cell

Precursor cell of a dendritic cell is a cell expressing CD14 (CD14⁺).

Auto-Antigens

An auto-antigen (auto-Ags), also called immunodominant peptide is usually a normal protein or complex of proteins that is recognized by the immune system of patients suffering of autoimmune diseases. Under normal conditions, these antigens do not promote immune responses, but in autoimmune diseases, these antigens promote T cell responses that result in tissue damages. A list of known immunodominant peptides is provided in Table 2.

Auto-Ags include autoAgs in T1D that comprise non-specific islet cell Ags (ICA), insulin, glutamic acid decarboxylase 65 (GAD65), insulinoma antigen-2 (IA-2), heat shock protein (HSP), islet-specific glucose-6-phosphatase catalytic subunit related protein (IGRP), imogen-38, and 13 cell-specific autoAgs, e.g., zinc transporter-8 (ZnT8), pancreatic duodenal homeobox factor 1 (PDX1), chromogranin A (CHGA), and islet amyloid polypeptide (IAPP); autoAgs in MS include myelin basic protein (MBP); proteolipid protein (PLP); myelin oligodendrocyte glycoprotein (MOG); myelin-associated antigen (MAG), myelin-associated oligodendrocyte basic protein (MOBP), and 2′,3′-cyclic-nucleotide 3′-phosphodiesterase (CNPase); S10013 protein, and transaldolase H; autoAgs in RA include Fc-part of immunoglobulins; Citrullinated antigens, Carbamylated antigens, collagen, 65-kDa heat-shock protein, cartilage glycoprotein-aggrecan G1, aggrecan core protein precursor (ACAN), α-fibrinogen (FGA), vimentin (VIM); autoAgs in IBD include zymogen granule membrane glycoprotein 2 (GP2); tropomyosins (TMs), carcinoembryonic antigen (CEA); autoAgs in vasculitis are Beta-2-glycoprotein 1 (b2GPI), Myeloperoxidase (MPO); Proteinase 3/Myeloblastin (PR3); autoAgs in myastenia gravis are nicotinic acetylcholine receptor (nAChR, muscle specific kinase (MuSK); autoAgs in autoimmune uvetitis retinal S-antigen (PDSAg), heterogeneous nuclear ribonucleoprotein H3 (Hnrph3), interphotoreceptor retinoid-binding protein (IRBP), cellular retinaldehyde-binding protein (cRALBP); autoAgs in Pemphigus vulgaris are in Desmoglein-31 (Dsg1), Desmoglein-3 (Dsg3), Pemphaxin (PX).

TABLE 2
known immunodominant peptides

| | Immunodominant peptides | | | |
|---|---|---|---|---|
| Disease | Name | Sequence | References | UniProtKB |
| Type 1 | InsB₉₋₂₃ | SHLVEALYLVCGERG (SEQ ID NO: 1) | (53) | P01308 |
| Diabetes | InsB9-23_{R22E} | SHLVEALYLVCGEEG (SEQ ID NO: 2) | | (INS_HUMAN) |
| | InsB923_{14E21E22E} | SHLVEELYVLVCGEEG (SEQ ID NO: 3) | | |
| | HIP-1 | GQVELGGGNAVEVLK (SEQ ID NO: 4) | (54) | |
| | HIP-2 | LQVELGGGPGAGSLQ (SEQ ID NO: 5) | | P01308 |
| | | | | (INS_HUMAN)/P01 |
| | | | | 308 (INS HUMAN)/ |
| | PPI_{C19-A3} | GSLQPLALEGSLQKRGIV (SEQ ID | (55) | P01308 |
| | | NO: 6) | | (INS_HUMAN) |
| | PIP₁₇₋₂₄ | WGPDPAAA (SEQ ID NO: 7) | (56) | Q9UGI5 |
| | InsB₃₋₂₃ | SHLVEALVLVCGERG (SEQ ID NO: 8) | (57) | (Q9UG15_HUMAN) |
| | GAD₁₁₄₋₁₂₃ | VMNILLQYVV (SEQ ID NO: 9) | | |
| | GAD65₃₃₅₋₃₅₂ | TAGTTVYGAFDPLLAVAD (SEQ ID | (58) | |
| | | NO: 10) | | |
| | GAD65₅₅₄₋₅₇₅ | VNFFRMVISNPAATHQDIDFLI (SEQ ID | | |
| | | NO: 11) | | |
| | IA-2₂₀₆₋₂₁₄ | VIVMLTPLV (SEQ ID NO: 12) | (57) | Q96T92 |
| | | | | (INSM2_HUMAN) |
| | IA-2₈₅₃₋₈₇₂ | SFYLKNVQTQETRTLTQFHF (SEQ ID | (58) | |
| | | NO: 13) | | |
| | IGRP₁₃₋₂₉ | QHLQKDYRAYYTF (SEQ ID NO: 14) | (59) | Q9NQR9 |
| | IGRP₂₃₋₃₉ | YTFLNFMSNVGDP (SEQ ID NO: 15) | | (G6PC2_HUMAN) |
| | IGRP₂₂₆₋₂₃₈ | RVLNIDLLWSVPI (SEQ ID NO: 16) | | |
| | IGRP₂₄₇₋₂₅₉ | DWIHIDTTPFAGL (SEQ ID NO: 17) | | |
| | ChgA₃₄₂₋₃₅₅ | WSKMDQLAKELTAE (SEQ ID NO: 18) | (58) | P10645 |
| | | | | (CMGA_HUMAN) |
| | ZnT8₁₈₆₋₁₉₄ | VAANIVLTV (SEQ ID NO: 19) | (60) | Q8IWU4 |
| | | | | (ZNT8_HUMAN) |
| | ZnT8₈₋₂₂ | MEFLERTYLVNDKAAKMHAF (SEQ ID | (61) | |
| | | NO: 20) | | |
| | ZnT8₁₉₋₂₉ | YLVNDKAAKMHAFTLESVEL (SEQ ID | | |
| | | NO: 21) | | |
| | ZnT8₁₂₉₋₁₃₄ | SLWLSSKPPSKRLTFGWHRA (SEQ ID | | |
| | | NO: 22) | | |
| | ZnT8₁₃₄₋₁₄₈ | PPSKRLTFGWHRAEILGALL (SEQ ID | | |
| | | NO: 23) | | |
| | ZnT8₂₆₀₋₂₇₄ | FIFSILVLASTITILKDFSI (SEQ ID NO: 24) | | |
| | ZnT8₂₆₇₋₂₈₁ | LKDFSILLMEGVPKSLNYSG (SEQ ID | | |
| | | NO: 25) | | |
| | ZnT8₂₉₅₋₃₀₉ | SLNYSGVKELILAVDGVLSV (SEQ ID | | |
| | | NO: 26) | | |
| | ZnT8₁₉₉₋₁₆₉ | FGWHRAEILGALLSILCIWV (SEQ ID | | |
| | | NO: 27) | | |
| | ZnT8₃₂₃₋₃₃₇ | TMNQVILSAHVATAASRDSQ (SEQ ID | | |
| | | NO: 28) | | |
| | HSP60₃₁₋₉₀ | KFGADARALMLQGVDLLADA (SEQ ID | (62) | P10809 |
| | | NO: 29) | | (CH60_HUMAN) |
| | HSP60₁₃₆₋₁₉₉ | NPVEIRRGVMLAVDAVIAEL (SEQ ID | | |
| | | NO: 30) | | |
| | HSP60₂₅₅₋₂₇₅ | QSIVPALEIANAHRKPLVIIA (SEQ ID | | |
| | | NO: 31) | | |
| | H5P60₂₈₆₋₃₀₅ | LVLNRLKVGLQVVAVKAPGF (SEQ ID | | |
| | | NO: 32) | | |
| | HSP60₄₃₆₋₄₅₅ | IVLGGGCALLRCIPALDSLT (SEQ ID | | |
| | | NO: 33) | | |
| | H5P60₅₁₁₋₉₃₉ | VNMVEKGIIDPTKVVRTALL (SEQ ID | | |
| | | NO: 34) | | |
| | Imogen₅₅₋₇₀ | SPSLWEIEFAKQLASV (SEQ ID NO: 35) | (63) | |
| Reumatoid | FGA ₇₉₋₉₁ | QDFTNRINKLKNS (SEQ ID NO: 36) | (64) | P02671 |
| Arthritis | | QDFTNCitINKLKNS (SEQ ID NO: 37) | | (FIBA_HUMAN) |
| | ACAN₈₄₋₁₀₃ | VVLLVATEGRVRVNSAYQDK (SEQ ID | | P16112 |
| | | NO: 38) | | (PGCA_HUMAN) |
| | | VVLLVATEGCitVRVNSAYQDK | | |
| | | (SEQ ID NO: 39) | | |
| | VIM₆₆₋₇₈ | SAVRARSSVPGVR (SEQ ID NO: 40) | | P08670 |
| | | SAVRACitSSVPGVR (SEQ ID NO: 41) | | (VIME_HUMAN) |
| | CII ₁₂₃₇₋₁₂₄₉ | QYMRADQAAGGLR (SEQ ID NO: 42) | | |
| | | QYMCitADQAAGGLR (SEQ ID NO: 43) | | P02458 |
| | | | | (CO2A1_HUMAN) |
| | CII ₂₆₁₋₂₇₃ | AGFKGEQGPKGEP (SEQ ID NO: 44) | (65) | |
| | CII ₂₆₁₋₂₇₃ with | | | |
| | K_{264/270} | | | |
| | CII ₂₆₁₋₂₇₅ | AGFKGEQGPKGEP (SEQ ID NO: 44) | (66) | |
| | | AGFKGgGEQGPKGEP (SEQ ID NO: 45) | | |
| Multiple | MOG₃₅₋₅₅ | MEVGWYRPPFSRVVHLYRNGK | (67) | Q16653 |
| Sclerosis | | (SEQ ID NO: 46) | | (MOG_HUMAN) |
| | PLP₁₃₉₋₁₅₄ | HCLGKWLGHPDKF (SEQ ID NO: 47) | | P60201 |
| | | | | (MYPR_HUMAN) |
| | MBP₈₃₋₉₇ | ENPVVHFFKNIV-TPR (SEQ ID NO: 48) | (68) | |
| | | | | P02686 |
| | | | | (MBP_HUMAN) |
| | MBP₁₃₋₃₂ | KYLATASTMDHARHGFLPRH (SEQ ID | (67) | |
| | | NO: 49) | | |
| | MBP₁₁₁₋₁₂₉ | LSRFSWGAEGQRPGFGYGG | | |
| | | (SEQ ID NO: 50) | | |
| | MBP₁₄₆₋₁₇₀ | AQGTLSKIFKLGGRDSRSGSPMARR | | |
| | | (SEQ ID NO: 51) | | |
| IBD | CAP1-6D | YLSGADLNL (SEQ ID NO: 52) | | Q13982 |
| | CEA₁₇₇₋₁₈₉ | LWWVNNQSLPVSP (SEQ ID NO: 53) | | (Q13982_HUMAN) |
| Celiac | α-gliadin ₅₇₋₇₄ | QLQPFPQPELPYPQPQP (SEQ ID | (69)} | Q41529 |
| Disease | | NO: 54) | | (Q41529 WHEAT) |
| | α-gliadin ₁₂₃₋₁₃₂ | QLIPCMDVVL (SEQ ID NO: 55) | | |
| | α-gliadin ₅₆₋₇₁ | YLQLQPFPQPQLPYP (SEQ ID NO: 56) | (70) | |
| | α-gliadin ₆₁₋₇₅ | PFPQPQLPYPQPQLP (SEQ ID NO: 57) | | |
| | α-gliadin ₆₆₋₈₀ | QLPYPQPQLPYPQPQ (SEQ ID NO: 58) | | |
| | α-gliadin ₇₁₋₈₅ | QPQLPYPQPQLPYPQ (SEQ ID NO: 59) | | |
| | α-gliadin ₇₆₋₉₀ | YPQPQLPYPQPQPFR (SEQ ID NO: 60) | | |
| | α-gliadin ₂₂₆₋₂₄₀ | YPSGQGSFQPSQQNP (SEQ ID NO: 61) | | |
| | α-gliadin ₂₃₁₋₂₄₅ | GSFQPSQQNPQAQGS (SEQ ID NO: 62) | | |
| | α-gliadin ₂₄₁₋₂₅₅ | QAQGSVQPQQLPQFE (SEQ ID NO: 63) | | |
| | α1-gliadin | QLQPFPQPELPY (SEQ ID NO: 64) | | |
| | α2-gliadin | PQPELPYPQPE (SEQ ID NO: 65) | (71) | |
| | ωl-gliadin | QQPFPQPEQPFP (SEQ ID NO: 66) | | |
| | ω2-gliadin | FPQPEQPFPWQP (SEQ ID NO: 67) | | |
| | γ2-gliadin | QGIIQPEQPAQL (SEQ ID NO: 68) | | |
| | ala-gliadin | SGEGSFQPSQENPQ (SEQ ID NO: 69) | (72) | |
| | γlb-gliadin | FPEQPEQPYPEQ (SEQ ID NO: 70) | | |
| CA | β2GPI₂₇₆₋₂₉ | KVSFFCKNKEKKCSY (SEQ ID NO: 71) | (73) | P02749 |
| PS | β2GPI₂₄₇₋₂₆₁ | VPVKKATVVYQGERV (SEQ ID NO: 72) | | (APOH_HUMAN) |
| | β2GPI₂₄₄₋₂₆₁ | SCKLVPVKKATVVYQGERVKIQ (SEQ | (74) | |
| | | ID NO: 73) | | |
| | β2GPI₁₋₂₀ | MISPVLILFSSFLCHVIAG (SEQ ID | (75) | |
| | | NO: 74) | | |
| Phemphigus | DG3₇₈₋₉₄ | QATQKITYRISGVGIDQ (SEQ ID NO: 75) | (76) | P32926 |
| Vulgaris | DG3₉₆₋₁₁₂ | PFGIFVVDKNTGDINIT (SEQ ID NO: 76) | | (DSG3_HUMAN) |
| | DG3₁₈₉₋₂₀₅ | HLNSKIAFKIVSQEPAG (SEQ ID NO: 77) | | |
| | DG3₂₀₅₋₂₂₁ | GTPMFLLSRNTGEVRTL (SEQ ID | | |
| | | NO: 78) | | |
| | DG3₂₅₀₋₂₆₆ | QCECNIKVKDVNDNFPM (SEQ ID | | |
| | | NO: 79) | | |
| | DG3₃₄₂₋₃₅₈ | SVKLSIAVKNKAEFHQS (SEQ ID | | |
| | | NO: 80) | | |
| | DG3₃₇₆₋₃₉₂ | NVREGIAFRPASKTFTV (SEQ ID | | |
| | | NO: 81) | | |
| | EC2/INT6₂₁₁₋₂₃₀ | IYVNVEPTFQRTLHKTK (SEQ ID NO: 82) | (77) | Q02413 |
| | EC2/INT6₂₁₆₋₂₃₅ | GEIRTMNNFLDREIYVNVEP (SEQ ID | | (DSGl_HUMAN) |
| | | NO: 83) | | |
| | EC2/INT6₂₂₁₋₂₄₀ | MNNFLDREIYNVEPTFQRT (SEQ ID | | |
| | | NO: 84) | | |
| | EC2/INT6₂₂₆₋₂₄₅ | DREIYVNVEPTFQRTLHKTK (SEQ ID | | |
| | | NO: 85) | | |
| Autoimmune | hS-Ag₂₈₁₋₃₀₀ | TLTLLPLLANNRERRGIALD (SEQ ID | (78) | P10523 |
| Uveitis | | NO: 86) | | (ARRS_HUMAN) |
| | hS-Ag₂₉₁₋₃₁₀ | NRERRGIALDGKIKHEDTL (SEQ ID | | |
| | | NO: 87) | | |
| | hS-Ag₂₈₇₋₃₀₆ | LLANNRERRGIALDGKIKHE (SEQ ID | | |
| | | NO: 88) | | |
| | hS-Ag₃₁₁₋₃₃₀ | ASSTIIKEGIDRTVLGILVS (SEQ ID | | |
| | | NO: 89) | | |
| | hS-Ag₃₃₁₋₃₅₀ | YQIKVKLTVSGFGELTSSE (SEQ ID | | |
| | | NO: 90) | | |
| | hS-Ag₁₋₂₀ | MAASGKTSKSEPNHVIFKK (SEQ ID | (79) | |
| | | NO: 91) | | |
| | hS-Ag₄₁₋₆₀ | QVQPVDGVVLVDPDLVKGKK (SEQ ID | | |
| | | NO: 92) | | |
| | hS-Ag₆₁₋₈₀ | VYVTLTCAFRYGQEDVDVIG (SEQ ID | | |
| | | NO: 93) | | |
| | hS-Ag₈₁₋₁₀₀ | LTFRRDLYFSRVQVYPPVGA (SEQ ID | | |
| | | NO: 94) | | |
| | hS-Ag₁₂₁₋₁₄₀ | PFLLTFPDYLPCSVMLQPAP (SEQ ID | | |
| | | NO: 95) | | |
| | hS-Ag₁₄₁₋₁₆₀ | QDSGKSCGVDFEVKAFATDS (SEQ ID | | |
| | | NO: 96) | | |
| | hS-Ag₁₆₁₋₁₈₀ | TDAEEDKIPKKSSVRYLIRS (SEQ ID | | |
| | | NO: 97) | | |
| | hS-Ag₂₀₁₋₂₂₀ | FMSDKPLHLAVSLNREIYFH (SEQ ID | | |
| | | NO: 98) | | |
| | hS-Ag₂₂₁₋₂₄₀ | GEPIPVTVTVTNNTEKTVKK (SEQ ID | | |
| | | NO: 99) | | |
| | hS-Ag₂₄₁₋₂₆₀ | IKACVEQVANVVLYSSDYYV (SEQ ID | | |
| | | NO: 100) | | |
| | hS-Ag₃₀₁₋₃₂₀ | GKIKHEDTNLASSTIIKEGI (SEQ ID | | |
| | | NO: 101) | | |
| | hS-Ag₃₄₄₋₃₅₆ | GELTSSEVATEVP (SEQ ID NO: 102) | | |
| | hS-Ag₃₄₆₋₃₅₆ | LTSSEVATEVP (SEQ ID NO: 103) | | |
| Myastenia | AChR₁₂₋₄₉ | FKDYSSVVRPVEDHRQVVEVTVGLQLI | (80) | P02708 |
| Gravis | | QLINVDEVNQI (SEQ ID NO: 104) | | (ACHA_HUMAN) |
| | AChR₄₈₋₆₇ | LGIWTYDGSVVAINPES (SEQ ID | | |
| | | NO: 105) | | |
| | AChR₇₅₋₁₁₅ | VKKIHIPSEKIWRPDLVLYNNADGDFAIV | | |
| | | KFTKVLLQYTGH (SEQ ID NO: 106) | | |
| | AChR₇₈₋₉₃ | IHIPSEKIWRPDLVLY (SEQ ID NO: 107) | | |
| | AChR₁₄₆₋₁₆₂ | LGIWTYDGVVAINPES (SEQ ID | | |
| | | NO: 108) | | |
| | AChR₁₉₅₋₂₁₂ | DPTYLDITYHFVMQRLPL (SEQ ID | | |
| | | NO: 109) | | |
| | AChR₂₄₀₋₂₅₇ | DTPYLDITYHFVMQRLPL (SEQ ID | (81) | |
| | | NO: 110) | | |
| | AChR₃₀₄₋₃₁₆ | VIVELIPSTSSAV (SEQ ID NO: 111) | | |
| | AChR₁₂₅₋₁₄₇ | KSYCEIIVTHFPFDEQNCSMKLG (SEQ | | |
| | | ID NO: 112) | | |
| Vasculitis | MPO₄₀₉₋₄₂₈ | PRWNGEKLYQEARKIVGAMV (SEQ ID | (82) | P05164 |
| | | NO: 113) | | (PERM_HUMAN) |
| | cPR3₁₃₈₋₁₆₉ | DLGWGVVGTHAAPAHGQALGAVGHW | (83) | P24158 |
| | | LVLLWQL (SEQ ID NO: 114) | | (PRTN3_HUMAN) |

Variants of such known immunodominant peptides are also included in the present invention. The variant maintains the antigenic properties of the immunodominant peptides.

Non-Harmful Antigens

Non-harmful antigens are substances present in the body and usually do not promote active immune responses (food antigens including gliadin, ovalbumin, peanut derived proteins, milk derived proteins, wheal derived proteins, ect.).

Allergens

An allergen is a usually harmless substance capable of triggering an immune response and results in an allergic reaction. Allegens include cereals containing gluten, peanut-derived proteins, timothy grass allergens (Phl p 1, 2, 5a, 5b, 6), been venom derived proteins, Bet v 1 of birch pollen (Betula verrucosa), Der p 1 and Derp 2 of house dust mite (Dermatophagoides pteronyssinus), Pyr c 5 of pear (Pyrus communis), and Cor a 1 of hazelnut (Corylus avellana).

Modulation of CD4+ and CD8+ T Cell Responses

Modulation of CD4+ and CD8+ T cell responses refers to effects on the ability of T cells to produce different levels of pro-inflammatory (i.e. IFN-g, IL-2, GM-CSF) or anti-Inflammatory (i.e. IL-10, TGF-β) cytokines, granzymes, and express receptors (i.e. CD69, CD25, CTLA-4). The level of pro-inflammatory and anti-Inflammatory cytokines may be measured by any method know in the art.

Modulation of Antigen-Specific CD4⁺ and CD8⁺ T Cell Proliferation In Vitro and/or In Vivo

Modulation of antigen-specific CD4⁺ and CD8⁺ T cell proliferation in vitro and/or in vivo is referring to a property of a cell to inhibit activation and proliferation of T cells.

Generation of Regulatory DC

Generation of regulatory DC refers to a method to modulate DC in order to render it able to secrete high levels of anti-inflammatory cytokines (i.e. IL-10) and low amount of pro-inflammatory cytokines (i.e. IL-12, TNF-α, ect), and to express tolerogenic molecules (i.e. HLA-G, ILT4, IDO).

Favoring the Expansion of Antigen-Specific Tr1 and/or FOXP3⁺ Treg Cells

Favoring the expansion of antigen-specific Tr1 and/or FOXP3⁺ Treg cells refers to a property of a cell to induce/convert CD4 T cells with pathogenic activity to a regulatory cell able to suppress T cell responses in vitro and/or in vivo.

Tolerogenic Cell

A tolerogenic cell is a cell that promotes the generation of regulatory cells in vitro and/or in vivo.

Antigen Presentation in the Context of Both MHC Class I and Class II

Presenting antigen in the context of both MHC class I and class II is a property of a cell to activate CD4⁺ and CD8⁺ T cells in an antigen-specific manner via their TCR.

Immunotherapeutic Agents

They are a class of molecules able to treat disease by inducing, enhancing, or suppressing an immune-responses, among other rapamycin, dexamethasone, vitamin D3, ect.

Cell Nomenclature

LV-DC is a dendritic cell than have been transduced with a lentiviral vector (LV).

tolDC is a dendritic cell that has tolerogenic activity.

LV.IiOVA is LV encoding for invariant chain fused with OVA peptide.

LV.OVA.miRNA a monodirectional LV encoding for invariant chain fused with OVA peptide and target sequences for miRNA155 and miRNA146a.

LV-IL-10/OVA is a bidirectional LV co-encoding for invariant chain fused with OVA peptide and IL-10.

LV-IDO/OVA is a bidirectional LV co-encoding for invariant chain fused with OVA peptide and IDO.

DC-OVA is a dendritic cell that has been transduced with a LV encoding for invariant chain fused with OVA peptide (LV-IiOVA).

DC-OVAmiRNA is a dendritic cell that has been transduced with a LV encoding for invariant chain fused with OVA peptide and target sequences for miRNA155 and miRNA146a.

DC-IL-10/OVA is a dendritic cell that has been transduced with a LV co-encoding for invariant chain fused with OVA peptide and IL-10 (LV-IL-10/OVA).

DC-IDO/OVA is a dendritic cell that has been transduced with a LV co-encoding for invariant chain fused with OVA peptide and IDO (LV-IDO/OVA).

OTII CD4⁺ T cells is a CD4⁺ T cells isolated from a TCR transgenic mice that recognize OVA₃₂₃₋₃₃₉ peptide.

OTI CD8⁺ T cells is a CD8⁺ T cells isolated from a TCR transgenic mice that recognize OVA₂₄₂₋₃₅₃ peptide.

DC pulsed with OVA peptide is a dendritic cell that has been pulsed with OVA peptide.

DC-UnT is a dendritic cell not transduced.

DC-GFP or DC^{GFP} is a dendritic cell that has been transduced with a LV encoding GFP (LV.GFP).

DC-InsB is a dendritic cell that has been transduced with a LV encoding for invariant chain fused with InsB (LV.InsB).

DC-InsB.miRNA is a dendritic cell that has been transduced with a LV encoding for invariant chain fused with InsB and target sequences for miRNA155 and miRNA146a (LV.InsB.miRNA).

DC-IL-10/InsB is a dendritic cell that has been transduced with a LV encoding for invariant chain fused with InsB peptide and IL-10 (LV.IL-10/InsB).

DC-IDO/InsB is a dendritic cell that has been transduced with a LV encoding for invariant chain fused with InsB and IDO (LV.IDO/InsB).

LV-ΔNGFR/GFP is a bidirectional LV co-encoding for ΔNGFR and GFP.

LV-GFP is a monodirectional LV encoding for GFP.

LV-IL-10 is a bidirectional LV co-encoding for ΔNGFR and IL-10.

DC^{IL-10} is a dendritic cell that has been transduced with a LV encoding for ΔNGFR and IL-10.

DC-10 is a dendritic cell that has been differentiated from CD14⁺ cells in the presence of IL-10, IL-4 and GM-CSF.

Allogeneic CD3⁺ T cells are T cells specific for alloAgs.

Allo-specific anergic CD4⁺ T cells are CD4⁺ T cells specific for alloAgs that do not proliferate.

Mature DC (mDC) is a dendritic cell that has been differentiated from CD14⁺ cells in the presence of IL-4 and GM-CSF and activated with LPS.

Allo-mDC is a dendritic cell that has been differentiated from allogeneic CD14⁺ cells in the presence of IL-4 and GM-CSF and activated with LPS.

Allo-specific IL-10-producing Tr1 Cells are T cells specific for alloAgs that produce IL-10 and express CD49b and LAG-3, are anergic and suppress T cell responses.

LV-ΔNGFR/Ag is a bidirectional LV co-encoding for invariant chain fused with antigen-derived peptide and ΔNGFR.

LV-IL-10/Ag is a bidirectional LV co-encoding for invariant chain fused with antigen-derived peptide and IL-10.

LV-CLIP is a bidirectional LV co-encoding for invariant chain CLIP peptide and ΔNGFR.

DC-IDO/Ag is a dendritic cell that has been transduced with a LV encoding for invariant chain fused with antigen-derived peptide and IDO.

hLV-DC is a dendritic cell that has been differentiated from human CD14⁺ cells and transduced with LV.

DC^{UT} is a dendritic cell that has been differentiated from allogeneic CD14⁺ cells in the presence of IL-4 and GM-CSF.

T(DC^{UT}) cells T cells that have been generated by culturing CD4⁺ T cells with allogeneic DC^{UT} for 10 days.

T(DC^{GFP}) T cells that have been generated by culturing CD4⁺ T cells with allogeneic DC^{GFP} for 10 days.

T(DC^{IL-10}) T cells that have been generated by culturing CD4⁺ T cells with allogeneic DC^{IL-10} for 10 days.)

T(stimDC^{IL-10}) T cells that have been generated by culturing CD4⁺ T cells with allogeneic DC^{IL-10} stimulated with LPS or Poli I:C for 10 days.

In some aspect, the disclosure provides transfected or transduced host cells. The term “transfection” or “transduction” is used to refer to the uptake of foreign DNA by a cell, and a cell has been “transfected” or “transduced” when exogenous DNA has been introduced inside the cell membrane. A number of transfection/transduction techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13: 197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of a DNA construct, a plasmid, an accessory function vector, or other transfer DNA associated with the production of lentivectors. The term includes the progeny of the original cell which has been transfected/transduced. Thus, a “host cell” as used herein may refer to a cell which has been transfected/transduced with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” or “genetically modified cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors, preferably lentiviral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In the present invention the term “indolamine dioxygenase” or “IDO” means IDO1 (indoleamine 2,3-dioxygenase, EC 1.13.1 1.52) or IDO2 (indoleamine-pyrrole 2,3 dioxygenase-like 1, EC 1.13.11.-) these being two different proteins that can catabolize tryptophan and can be expressed by APCs.

“Immune tolerance” means the lack of response to antigens (self- or foreign-antigens) and included natural tolerance or induced tolerance (i.e. deliberate manipulation of the immune system).

“Self-antigen” means any molecule or chemical group of an organism which acts as an antigen in inducing a T effector cell response or antibody formation in another organism but to which the healthy immune system of the parent organism is tolerant. Under certain circumstances, for example, when a subject is suffering from or is susceptible to an autoimmune disease, the parent organism is not tolerant to the self-antigen and a specific adaptive immune response is mounted against self-antigens.

“Exogenous therapeutic agent” means any therapeutic agent for treatment of a subject that originates from outside the subject.

The term “co-culturing” means culturing two (or more) cell types in the presence of each other.

The skilled artisan will understand that the compositions and methods described herein can be used, in conjunction with current therapeutic approaches for treating the diseases and disorders described elsewhere herein. By way of non-limiting example, the cells of the present invention can be used in conjunction with the use of immunosuppressive drug therapy. An advantage of using the cells in conjunction with immunosuppressive drugs is that by using the methods of the present invention to ameliorate the severity of the immune response in a subject, such as a transplant recipient, the amount of immunosuppressive drug therapy used and/or the frequency of administration of immunosuppressive drug therapy can be reduced. A benefit of reducing the use of immunosuppressive drug therapy is the alleviation of general immune suppression and unwanted side effects associated with immunosuppressive drug therapy. It is also contemplated that the cells of the present invention may be administered into a recipient repeatedly or as a “one-time” therapy for the prevention or treatment of a disease or disorder, such as an autoimmune disease or disorder, an inflammatory disease or disorder, or a disease or disorder associated with transplant, such as host rejection of donor tissue or graft versus host disease. A one-time administration of cells into the recipient of the transplant eliminates the need for chronic immunosuppressive drug therapy. However, if desired, multiple administrations of cells may also be employed.

Based upon the disclosure provided, herein, the dendritic cells or precursors thereof can be obtained from any source, for example, from the tissue donor, the transplant recipient or an otherwise unrelated source (a different individual or species altogether). The cells may be autologous with respect to the T cells (obtained from the same host) or allogeneic with respect to the T cells. In the case where the dendritic cells or precursor thereof are allogeneic, the cells may be autologous with respect to the transplant to which the T cells are responding to, or the cells may be obtained, from a mammal that is allogeneic with respect to both the source of the T cells and the source of the transplant to which the T cells are responding to. In addition, the T cells may be xenogeneic to the T cells (obtained from an animal of a different species), for example mouse cells may be used to suppress activation and proliferation of human T cells.

Another aspect of the present invention encompasses the route of administering the cells to the subject. Cells can be administered by a route that is suitable under the circumstances. Cells can be administered systemically, i.e., parenterally, by intravenous injection or intraperitoneal injection or can be targeted to a particular tissue or organ, such as bone marrow, cells can be administered via a subcutaneous implantation of cells or by injection of the cells into connective tissue, for example, muscle.

The cells can be suspended in an appropriate diluent, at a concentration of about 1×10⁴ to about 20×10⁷, preferably about 5×10⁶ cells/ml. Suitable excipients for injection solutions are those that are biologically and physiologically compatible with the cells and with the recipient, such as buffered saline solution or other suitable excipients. The composition for administration can be formulated, produced and stored according to standard methods complying with proper sterility and stability.

The dosage of the cells varies within wide limits and may be adjusted to the subject's requirements in each particular case. The number of cells used depends on the weight and condition of the recipient, the number and/or frequency of administrations, and other variables known to those of skill in the art.

Auto-Immune Disease

Auto-immune disease is a condition arising from an abnormal immune response against auto-antigens and comprises: type 1 diabetes mellitus, autoimmune enteropathy, rheumatoid arthritis, systemic lupus erythematosus, multiple sclerosis, autoimmune myositis, psoriasis, Addison's disease, Grave's disease, Sjogren's syndrome, Hashimoto's thyroiditis, myasthenia gravis, vasculitis, pernicious anemia, celiac disease, autoimmune hepatitis, alopecia areata, pemphigus vulgaris, vitiligo, aplastic anemia, autoimmune uveitis.

Auto-immune disease also includes: Alopecia Areata, Amyotrophic Lateral Sclerosis (Lou Gehrig's), Ankylosing Spondylitis, Anti-GBM Nephritis, Antiphospholipid Syndrome, Osteoarthritis, Asthma, Atopic Allergy, Atopic Dermatitis, Autoimmune Active Chronic Hepatitis, Autoimmune Inner Ear Disease (AIED), Balo Disease, Behcet's Disease, Berger's Disease, Bullous Pemphigoid, Cardiomyopathy, Chronic Fatigue Immune Dysfunction Syndrome, Churg Strauss Syndrome, Cicatricial Pemphigoid, Cold Agglutinin Disease, Colitis Cranial Arteritis, Crest Syndrome, Crohn's Disease, Dego's Disease, Dermatomyositis & JDM, Devic Disease, Eczema, Essential Mixed Cryoglobulinemia, Eoscinophilic Fascitis, Fibromyalgia—Fibromyositis, Fibrosing Alveolitis, Giant Cell Arteritis, Glomerulonephritis, Goodpasture's Disease, Guillain-Barre Syndrome, Hashimoto's Thyroiditis, Hepatitis, Hughes Syndrome, Idiopathic Pulmonary Fibrosis, Idiopathic Thrombocytopenic Purpura, Irritable Bowel Syndrome, Kawasaki's Disease, Lichen Planus, Lupoid Hepatitis, Lupus/SLE, Lyme Disease, Meniere's Disease, Mixed Connective Tissue Disease, Myositis/JM, JDM, & JA, Osteoporosis, Pars Planitis, Pemphigus Vulgaris, Polyglandular Autoimmune Syndromes, Polymyalgia Rheumatica, Polymyositis, Primary Biliary Cirrhosis, Primary Sclerosis Cholangitis, Psoriasis, Raynaud's Syndrome, Reiter's Syndrome, Rheumatic Fever, Rheumatoid Arthritis, Scleritis, Scleroderma, Sticky Blood Syndrome, Still's Disease, Stiff Man Syndrome, Sydenham's Chorea, Takayasus Arteritis, Temporal Arteritis, Ulcerative Colitis, Uveitis, Vasculitis, Wegener's Granulomatosis and Wilson's Syndrome.

Preferred autoimmune diseases include vasculitis such as catastrophic anti-phospholipid syndrome (also named Asherson's syndrome), Giant Cell Arteritis and anti-ANCA vasculitis, myasthemia gravis, refractory celiac disease, autoimmune uveitis such as Behcet's Disease, pemphigus vulgaris, giant cell myocarditis, Graves' disease, Addison's disease and granulomatosis with polyangiitis.

Material and Methods

Subjects. All protocols were approved by the Institutional Review Board and samples collected under written informed consent according to the Declaration of Helsinki.

Cell preparation and cell lines. Bone marrow cells isolated from Balb/c, C57Bl/6 or NOD mice were kept in culture for 8 days the presence of rmGM-CSF (25 ng/mL; R&D Systems) to differentiate into DC.

Peripheral blood mononuclear cells (PBMC) were prepared by centrifugation over gradients. CD4⁺ T cells were purified with the CD4 T cell isolation kit (Miltenyi Biotec), resulting purity of >95%. CD4⁺ T cells were then depleted of CD45RO⁺ cells using anti-CD45RO-coupled magnetic beads and LD negative selection columns (Miltenyi Biotech). The proportion of CD4⁺ CD45RA⁺ in the selected population was consistently greater than 90%. CD14⁺ and CD3⁺ T cells were purified by positive selection with CD14⁺ and CD3⁺ Microbeads (Miltenyi Biotec), respectively with a resulting purity of >95%.

CD14⁺ monocytes were isolated from PBMC by positive selection using CD14 MicroBeads (Miltenyi Biotech) according to the manufacturer's instructions. Cells were cultured in RPMI 1640 (Lonza) supplemented with 10% Fetal Bovine Serum (FBS) (Lonza,) or with 5% Human Serum (HS) (EuroClone), 100 [U/ml] penicillin/streptomycin (Lonza, Italy), 2 mM L-Glutamine (Lonza, Italy), (DC medium) at 37° C. in the presence of 10 ng/ml rhIL-4 (R&D Systems) and 100 ng/ml rhGM-CSF (Genzyme) with 10 ng/ml of rhIL-10 (BD, Bioscience) for 7 days to differentiate DC-10. Cells cultured with rhIL-4 and rhGM-CSF on day 5 were matured with 1 μg/ml of LPS (Sigma) for additional 2 days to generate mDC. At day 7, DC were collected, phenotypically analyzed, and used to stimulate T cells.

In some experiments HLA-DQ8⁺ or HLA-DQ2.5⁺ CD14⁺ cells were cultured with serum-free DC medium (CellGenix) supplemented with 100 [U/ml] penicillin/streptomycin (Euroclone) in the presence of 10 ng/ml rhIL-4 and 100 ng/ml rhGM-CSF (Miltenyi Biotec) with or without 10 ng/ml of rhIL-10 (CellGenix) at a density of 10\hat{ }6 cells/ml of culture medium. On Day 3 cells were supplemented with 1 ml of serum-free medium plus 20 ng/ml rhIL-4 and 200 ng/ml rhGM-CSF (Miltenyi Biotec). Immature DCs were collected on day 7 for subsequent phenotypical and functional analysis.

DNA extraction and HLA-DQ screening. To select HLA-DQ8⁺ and/or HLA-DQ2.5⁺ healthy donors, genomic DNA was extracted from 200p1 of whole blood using QIAamp DNA Blood Mini Kit (Qiagen), according to Manufacturer's instructions. Presence or absence of the HLA-DQ8 or -DQ2.5 allele was determined by PCR using Eu-GEN Kit (Eurospital), following Manufacturer's instructions.

Plasmid construction. The coding sequence of murine invariant chain (CD74) fused to sequences encoding for InsB₄₋₂₉ or OVA₃₁₅₋₃₅₃ was synthetized (GeneArt) and cloned into several LV backbones: hPGK.XXX.WPRE (84) to obtain LV-IiOVA and LV-IiInsB; hPGK.XXX.WPRE miR155T.mir146aT to obtain LV-IiOVAmiRNA and LV-IiInsBmiRNA and into bi-directional backbones hPGK.XXX.WPRE.mCMVIL10.SV40PA (85) and hPGK.XXX.WPRE.mCMVIDO.SV40PA to obtain LV-IL-10/OVA and LV-IL-10/InsB and LV-IDO/OVA and LV-IDO/InsB, respectively.

The coding sequence of human IL-10 was excised from pH15C (ATCC no 68192), and the 549 bp fragment was cloned into the multiple cloning site of pBluKSM (Invitrogen) to obtain pBluKSM-hIL-10. A fragment of 555 bp was obtained by excision of hIL-10 from pBluKSM-hIL-10 and ligation to 1074.1071.hPGK.GFP.WPRE.mhCMV.dNGFR.SV40PA (85) (here named LV-ΔNGFR), to obtain LV-IL-10/ΔNGFR. The presence of the bidirectional promoter (human PGK promoter plus minimal core element of the CMV promoter in opposite direction) allows co-expression of the two transgenes. The sequence of LV-IL-10/ΔNGFR was verified by pyrosequencing (Primm).

The coding sequence of p33 isoform of human invariant chain (Iip33) fused to a sequence encoding for the InsulinB peptide 4-29 (InsB₄₋₂₉) or a2-gliadin 51-80 was synthetized (GeneArt) and cloned into the following bi-directional backbones: hPGK.XXX.WPRE.mCMV.YYYY.SV40PA to obtain LV-Iip33Ag/ΔNGFR, LVIip33Ag/IL-10, or LVIip33Ag/IDO As control, the antigen-encoding sequence was replaced with the Class II-associated invariant chain peptide (CLIP). The sequence of the resulting plasmids was verified by pyrosequencing (GATC).

Vector production and titration. VSV-G-pseudotyped third generation bdLVs were produced by Ca₃PO₄ transient four-plasmid co-transfection into 293T cells and concentrated by ultracentrifugation as described (40). Titer was estimated by limiting dilution, vector particles were measured by HIV-1 Gag p24 antigen immune capture (NEN Life Science Products;), and vector infectivity was calculated as the ratio between titer and particle. Titers ranged from 5×10⁸ to 6×10⁹ transducing units/ml, and infectivity from 5×10⁴ to 10⁵ transducing units/ng of p24.

Transduction of dendritic cells. Bone marrow cells isolated from Balb/c, C57Bl/6 or NOD mice were differentiated into DC in the presence of rmGM-CSF (25 ng/mL; R&D Systems) and transduced with LV on day 2 at a multiplicity of infection (MOI) of 3.

CD14⁺ monocytes were plated as above described in the presence of Viral-Like-Particles (VIP) containing Viral Protein X (VPX) 1-5 μl. After 6 h LVs were added at a Multiplicity of Infection (MOI) of 5. After 14-18 h half medium was replenished. Efficiency of transduction cells was assessed on control transduced by flow cytometry based on cell surface expression of ΔNGFR.

Cytokine determination. Monocyte-derived DCs were collected at day 7, washed with PBS and re-plated at a density of 500000 cells/ml in fresh medium alone or supplemented with LPS 200 ng/ml and human IFNγ 50 ng/ml. After 48 h, supernatants were collected and cytokine concentration was determined by ELISA.

Proliferation and suppression assays. To assess Ag-specific proliferation of CD4+ and CD8+ T cells, OTII and OTI cells were labelled with eFluor-670 proliferation dye (Invitrogen), following Manufacturer's instructions. eFluor-labelled T cells were plated in U-bottom 96 well plates in a final volume of 200p1 alone or in the presence of LV-DC (T: DC ratio of 10:1). After 5 days, proliferation of T cells was assessed by flow cytometry. Cells were acquired using a BD-FACSCanto II analyzer and analyses were performed using Flow-Jo software.

To assess Ag-specific proliferation CD4+ T cells, autologous to monocyte-derived DCs, were thawed, rested for 1-2 h at 37° C. and labelled with efluor-450 proliferation dye (Invitrogen), following Manufacturer's instructions. 150000 eFluor-labelled CD4+ T cells were plated in 96 round-bottom well plates in a final volume of 200p1 alone or in the presence of DCs (T: DC ratio of 10:1) transduced with LV-Iip33Ag/ΔNGFR or LVIip33Ag/IL-10 or control LV-Iip33-CLIP. After 6 days, proliferation of CD4+ T cells was assessed by flow cytometry. Cells were acquired using a BD-FACSCanto II analyzer and analyses were performed using Flow-Jo software.

Flow cytometry analysis. Phenotype murine BM-LVDC was determined by flow cytometry on day 8 at the end of differentiation. For the detection of cell surface antigens, the following monoclonal antibodies (mAbs) were used: anti-CD11c-V450 (e-bioscience), anti-CD86-Pe-Cy7 (BD Biosciences), anti-CD80-PerCPCy5.5 (BD Biosciences), anti-IAb-PE (BD Biosciences). OTII cells were identified using anti-CD4-Pe-Cy7 (BD Biosciences) and anti-CD45.2-Pacific Blue (BD Biosciences).

Phenotype of monocyte derived human LV-DC was determined by flow cytometry on day 7. For the detection of cell surface antigens the following monoclonal antibodies (mAbs) were used: anti-DC-SIGN-Pe (BD Biosciences), -CD14-FITC (BD Biosciences), -HLADR-APC-Cy7 (BD Biosciences), CD86-PercP-Cy5.5 (BD Biosciences), CD83-BV421 (BD Biosciences), DNGFR-APC (Miltenyi Biotec), CD11c-PE-CY7 (BD Biosciences). Cell surface expression of tolerogenic molecules was also determined: anti-HLAG-PE (ExBio), -ILT4-APC (R&D Systems), -CD163-PcPCy5.5 (BD Biosciences), -CD141-BV421 (BD Biosciences). Cell vitality was assessed using LIVE/DEAD Cell Viability Assays (Thermo Fisher), according to Manufacturer's instructions. To assess the frequency of IL-10-producing DCs, LV-DCs were stimulated for 14-16 h with LPS 200 ng/ml and IFNg 50 ng/ml plus Brefeldin A (10 μg/ml). Intracellular expression of IL-10 was determined as previously described (Levings JI 2001), using anti-IL-10-Pe (BD Pharmingen). To assess the frequency of IDO-espressing DCs, intracellular staining with anti-human IDO-Pe (e-bioscience) was performed after 20 min fixation with 2% Formaldeyde solution (Thermo Fisher) and 10 min Permeabilization with PBS 2% FBS containing 0.5% Saponin (Sigma).

For the detection of FOXP3 (clone 259D, Biolegend, USA) after surface staining, cells were fixed, permeabilized, and stained with the Foxp3 staining Buffer Set according to the manufacturer's instructions (eBioscience, USA). For the expression of Granzyme B (clone MHGB04, Invitrogen, USA) after surface staining, cells were fixed, permeabilized, and stained with the BD Cytofix/Cytoperm™ Kit according to the manufacturer's instructions (Cat. No. 554714, Biolegend, USA). Samples were acquired using BD-FACSCantoII or BD-LSR Fortessa analyzers and analyses were performed using Flow-Jo software.

Mice. C57Bl/6, female NOD (NOD/LtJ) and Balb/c mice were purchased (Charles River Laboratories) and housed in specific pathogen-free conditions. The inventors crossed and generated Foxp3 reporter mice in the inventors' laboratory. The inventors used age- and sex-matched littermates between 8 and 12 weeks of age. Chimeric mice were generated by transplanting CD45.1 (95%) and CD45.2 OTII/FirTiger (5%) BM cells into lethally irradiated CD45.1 mice. OTII/FirTiger CD4⁺ T cells are TCR transgenic cells recognizing OVA₃₂₃₋₃₃₉ and expressing RFP and GFP as reporter genes for foxp3 and il10, respectively.

NOD mice were considered diabetic when blood glucose measurements were ≥250 mg/dl on two successive days as determined by a Bayer BREEZE Blood Glucose Monitoring System (Bayer). All procedures were reviewed and approved by the Institutional Animal Care and Use Committee (IACUC) at San Raffaele Institute, Milan (IUCAC 416 and 604).

GvHD model: Balb/c mice were lethally irradiated and intravenously injected with C57Bl/6 BM cells (10⁷) and splenocytes (5×10⁶). On day 2 mice were adoptively transferred with DC^{GFP} DC^{IL-10} (2×10⁶), Weight loss and survival of mice were monitored.

Method to generate human Treg cells in vitro. To induce Ag-specific CD4+ Treg cells, T cells autologous to monocyte-derived DCs, were thawed, rested for 1-2 h at 37° C. and labelled with efluor-450 proliferation dye (Invitrogen), following Manufacturer's instructions. 10⁶ cells eFluor-labelled CD4+ T cells were plated in 24 well plate in a final volume of 2 ml in the presence of DCs (T: DC ratio of 10:1) transduced with LV-Iip33Ag/DNGFR, LVIip33Ag/IL-10, LVIip33Ag/IDO, or control LV-Iip33-CLIP. After 10 days, proliferation of CD4⁺ T cells was assessed by flow cytometry and in case of LVIip33Ag/IL-10 the presence of TR1 cells was assess by the co-expression of CD49b and LAG-3, and in case of LVIip33Ag/IDO the presence of FOXP3+ Treg was assessed by the co-expression of FOXP3 and CTLA-4, on proliferating cells. Cells were acquired using a BD-FACSCanto II analyzer and analyses were performed using Flow-Jo software.

Vpx-VLP production. Concentrated Vpx-incorporating viral-like particles (VLPs) were produced by Ca₃PO₄ transient two-plasmids (VSV-G expressing plasmid and the Simian Immunodeficiency Virus-derived packaging plasmid SIV3+) into 293T cells and concentrated by ultracentrifugation as described (86). Titer was estimated by limiting dilution. Titers ranged from 5×10⁸ to 6×10⁹ transducing units/ml.

T cell differentiation and suppression assay. 10⁶ CD4⁺ T cells were cultured with 10⁵ allogeneic DC (10:1, T:DC) in X-VIVO 15 medium (Lonza, Switzerland), supplemented with 5% human serum (Sigma Aldrich, CA, USA), and 100 [U/ml] penicillin/streptomycin (Lonza, Switzerland). After 10 days, primed T cells were collected and purified using CD4 Microbeads (Miltenyi Biotech, Germany). T cells stimulated with DC^{UT} are referred to as T(DC^{UT}) cells, while those stimulated with DC^{GFP} as T(DC^{GFP}) cells. T cells cultured with unstimulated DC^{IL-10} are referred to as T(DC^{IL-10}) cells, while those cultured with LPS- or Poli I:C-stimulated DC^{IL-10} are referred to as T(stimDC^{IL-10}) cells.

Primed T cells were stained Cell Proliferation Dye eFluor® 670 (eBioscience, CA, USA) and then plated with DC^{UT} from the same donor used for priming (10:1, T:DC). After 3 days of stimulation, T cells were collected, washed, and proliferation was evaluated by flow cytometry. To evaluate the suppressive activity of T(DC^{IL-10}) and T(stimDC^{IL-10}) cells, we stained total CD4⁺ T cells (responder cells) autologous to T cells used in priming with Cell Proliferation Dye eFluor® 450 (eBioscience, CA, USA), and activated them with mature DC^{UT} from the same donor used for priming. T(DC^{IL-10}) or T(DC^{IL-10}*) cells stained with Cell Proliferation Dye eFluor® 670 were added at a 1:1 ratio with responder cells (total T:DC ratio is 10:1). After 4 days, the percentages of divided responder T cells were calculated by proliferation dye dilution by flow cytometer.

DC stimulation. In some experiments, DC were collected at day 7 and re-plated alone or in the presence of the following stimulation: 1 μg/ml of LPS (Sigma Aldrich, CA, USA), 10⁸ cells/ml of Heat Killed Listeria monocytogenes (code tlrl-hklm, InvivoGen, CA, USA), 1 ug/ml of Flagellin S. typhimurium (code tlrl-stfla, InvivoGen, CA, USA), 10 ug/ml of Poli (I:C) (code tlrl-pic InvivoGen, CA, USA, 5 uM of ODN2006 (CpG) (Code tlrl-2006, InvivoGen, CA, USA or a mix of 10 ng/ml for each cytokine of IL-1b, TNF-a and IL-6 (R&D Systems, MN, USA). After 24 hours, supernatants were collected to evaluate the cytokine secretion profile by ELISA, and cells were analysed by flow cytometry.

Modulation of immune response in humanized mice. 2-5 days old NSG (NOD.Cg-Prkdc^{scid} II2rg^{tm1}WjI/SzJ, JAX mouse strain) mice were sub-lethally irradiated (1.5 cGy) and injected intrahepatically 5-7 hours later with 10⁵ CD34+ (purity ≥95%, Lonza), as previously described (Santoni de Sio et al. JACI 2018).

Percentages of human total and T cells in peripheral blood were monitored by flow cytometry starting from 8 weeks post-transplant. Once human engraftment was stable and T cell repopulation clearly detectable (usually around 11-13 weeks post-transplant), huMice were immunized by intravenous injection of 5×10⁶ allogeneic CD3− cells, magnetically isolated (Dynabeads CD3—Thermo Fisher Scientific) from human peripheral blood. One week later, human T cell percentages were assessed by flow cytometry, huMice randomly assigned to experimental groups and injected with 3×10⁵ untransduced dendritic cells (DC^{UT}), or 3×10⁵ untransduced plus 3×10⁵ GFP or IL-10 transduced dendritic cells (DC^{GFP} and DC^{IL-10}, respectively), differentiated from CD14+ monocytes isolated from the same donor used for CD3-purification. T cell proliferation was assessed in peripheral blood by Ki67 staining.

Generation of packaging cell line CD47 hi and CD47 free. The Cas9 and sgRNA expressing plasmids previously described (87), were used to disrupt CD47 expression in 293T cells. The sequences of the CRISPR used to generate the sgRNA are: CD47A (CTACTGAAGTATACGTAAAGTGG) (SEQ ID NO:115), (CTTGTTTAGAGCTCCATCAAAGG) (SEQ ID NO:116), (ATCGAGCTAAAATATCGTGTTGG) (SEQ ID NO:117).

Gene disruption and mismatch-selective endonuclease assayGene disruption was performed by calcium phosphate-mediated transient transfection of the indicated amount of the desired sgRNA-expressing plasmid and the Cas9-expressing plasmid. The mismatch-selective endonuclease assay was used to measure the extent of mutations consequent to non-homologous end joining (NHEJ) at the Cas9 target sites, as described (88). PCR was performed using primers flanking the sgRNA binding site in in the CD47gene (fw: 5′-TTCCTTTCCAGGATCAGCTCAGC-3′(SEQ ID NO:118); rv: 5′-TTGATTCAAAGGAGTACCTATCCC-3′) (SEQ ID NO:119).

SIN RV genome transfer PGK.CD47 encoding for the gene synthesized human codon-optimized version of the CD47 cDNA (Genewiz) was exchanged with GFP into pRT43.3.PGK.GFP (BamHI-NotI)(89) for generating 293T CD47 high cells. 293T cells were transfected with pRT43.3.PGK.CD47, the packaging plasmid pCMV-Gag/Pol (Moloney Leukemia Virus), and pMD2.G, as described (89). 293T cells CD47 hi and CD47 free were used to generate LV as described above.

Statistical Analysis. Average values are reported as Mean±SEM. The inventors used Mann Whitney test and ANOVA test to determine the statistical significance of the data. The inventors defined significance as *P≤0.05, **P≤0.005, ***P≤0.0005, and ***P<0.0001. The inventors performed statistical calculations with the Prism program 5.0 (GraphPad Software, Inc.).

### SEQUENCES

In the following proteins, the immunodominant peptides or epitopes are highlighted in BOLD and deamidated residues are highlighted in in grey

LV Constructs for Murine DC

Invariant chain (m-li, CD74) (DNA)
(SEQ ID NO: 120)
atggatgaccaacgcgacctcatctctaaccatgaacagttgcccatactgggcaaccgccctagagagccagaaaggtgcagc
cgtggagctctgtacaccggtgtctctgtcctggtggctctgctcttggctgggcaggccaccactgcttacttcctgtaccagcaacag
ggccgcctagacaagctgaccatcacctcccagaacctgcaactggagagccttcgcatgaagcttccgaaatctgccaaacctgt
gagccagatgcggatggctactcccttgctgatgcgtccaatgtccatggataacatgctccttgggcctgtgaagaacgttaccaag
tacggcaacatgacccaggaccatgtgatgcatctgctcacgaggtctggacccctggagtacccgcagctgaaggggaccttcc
cagagaatctgaagcatcttaagaactccatggatggcgtgaactggaagatcttcgagagctggatgaagcagtggctcttgtttga
gatgagcaagaactccctggaggagaagaagcccaccgaggctccacctaaagagccactggacatggaagacctatcttctg
gcctgggagtgaccaggcaggaactgggtcaagtcaccctg
Invariant chain (m-li, CD74) (protein)
(SEQ ID NO: 121)
MDDQRDLISNHEQLPILGNRPREPERCSRGALYTGVSVLVALLLAGQATTAYFLYQQQGRLD
KLTITSQNLQLESLRMKLPKSAKPVSQMRMATPLLMRPMSMDNMLLGPVKNVTKYGNMTQD
HVMHLLTRSGPLEYPQLKGTFPENLKHLKNSMDGVNWKIFESWMKQWLLFEMSKNSLEEKK
PTEAPPKEPLDMEDLSSGLGVTRQELGQVTL
Invariant chain fused in frame to:
>OVA 315-353,STOP (DNA)
(SEQ ID NO: 122)
tgtggcatctcctcagcagagagcctgaagatatctcaagctgtccatgcagcacatgcagaaatcaatgaagcaggcagagagg
tggtagggtcagcagaggctggagtggatgctgcaagctga
OVA 315-353,STOP (protein) (epitope OVA₃₂₃₋₃₃₉)
(SEQ ID NO: 123)
CGISSAESLKISQAVHAAHAEINEAGREVVGSAEAGVDAAS*
>OVA 242-353.STOP (DNA)
(SEQ ID NO: 124)
tgcatgttggtgctgttgcctgatgaagtctcaggccttgagcagcttgagagtataatcaactttgaaaaactgactgaatggaccagt
tctaacgttatggaagagaggaagatcaaagtgtacttacctcgcatgaagatggaggaaaaatacaacctcacatctgtcttaatg
gctatgggcattactgacgtgtttagctcttcagccaatctgtctggcatctcctcagcagagagcctgaagatatctcaagctgtccat
gcagcacatgcagaaatcaatgaagcaggcagagaggtggtagggtcagcagaggctggagtggatgctgccagctga
OVA 242-353.STOP (protein) (epitopes: OVA₂₅₇₋₂₆₄; OVA₃₂₃₋₃₃₉)
(SEQ ID NO: 125)
CMLVLLPDEVSGLEQLESIINFEKLTEWTSSNVMEERKIKVYLPRMKMEEKYNLTSVLMAMGIT
DVFSSSANLSGISSAESLKISQAVHAAHAEINEAGREVVGSAEAGVDAAS*
>InsB 4-29.STOP
(SEQ ID NO: 126)
cagcacctttgtggttcccacctggtggaggctctctacctggtgtgtggggagcgtggcttcttctacacacccatgtaa
InsB 4-29.STOP (protein) (epitope InsB₉₋₂₃)
(SEQ ID NO: 127)
QHLCGSHLVEALYLVCGERGFFYTPM*
>InsB 4-29R22E.STOP (DNA)
(SEQ ID NO: 128)
cagcacctttgtggttcccacctggtggaggctctctacctggtgtgtggggagcgtggcttcttctacacacccatgtaa
InsB 4-29R22E.STOP (protein) (epitope InsB_{9-23R22E})
(SEQ ID NO: 129)
QHLCGSHLVEALYLVCGEEGFFYTPM*
>GAD65 500-585 (DNA)
(SEQ ID NO: 130)
Cacacaaatgtctgcttctggtttgtacctcctagtttgcgcactctggaagacaatgaagagagaatgagccgcctctcaaaggtgg
cgccagtgattaaagccagaatgatggagtatgggaccacaatggtcagctaccaacccttaggggacaaggtcaacttcttccgc
atggtcatctcaaaccctgcagcaactcaccaagacattgacttccttattgaagaaatcgaacgcctcggacaagatttgt
GAD65 500-585 (protein) (epitopes: GAD509-528; GAD ₅₂₄₋₅₄₃, GAD₅₆₁₋₅₇₅)
(SEQ ID NO: 131)
HTNVCFWFVPPSLRTLEDNEERMSRLSKVAPVIKARMMEYGTTMVSYQPLGDKVNFFRMVI
SNPAATHQDIDFLIEEIERLGQDL
>GAD65 202-225 (DNA)
(SEQ ID NO: 132)
actaacatgttcacctatgagatcgcccctgtatttgtgctgctagaatatgttacactaaagaaaatgagataa
GAD65 202-225 (protein) (epitope: GAD₂₀₆₋₂₂₀)
(SEQ ID NO: 133)
TNMFTYEIAPVFVLLEYVTLKKMR
>IGRP191-218(DNA)
(SEQ ID NO: 134)
gaggcctttgaacacactccaggagtccacatggccagcttgagtgtgtacctgaagaccaacgtcttcctcttcctgtttgcctaa
IGRP195-214(protein) (epitope: IGRP₁₉₅₋₂₁₄)
(SEQ ID NO: 135)
EAFEHTPGVHMASLSVYLKTNVFLFLFA
W1E14 (DNA)
(SEQ ID NO: 136)
gaggtggaggaccctcaggtggcccagctggagctgggcggcggccctggcgccggcgacctgcagaccctggccctgtggag
cagaatggaccagctggccaaggagctgaccgccgagtga
WE14 (fusion protein) (combination N-ter C pep - ChrA)
(SEQ ID NO: 137)
EVEDPQVAQLELGGGPGAGDLQTLAL- WSRMDQLAKELTAE
LV constructs for human DC
Human invariant chain (hu-li, p33, clip) (DNA) (SEQ ID NO: 138)
atggatgaccagcgcgaccttatctccaacaatgagcaactgcccatgctgggccggcgccctggggccccggagagcaagtgc
agccgcggagccctgtacacaggcttttccatcctggtgactctgctcctcgctggccaggccaccaccgcctacttcctgtaccagc
agcagggccggctggacaaactgacagtcacctcccagaacctgcagctggagaacctgcgcatgaagcttcccaagcctccca
agcctgtgagcaagatgcgcatggccaccccgctgctgatgcaggcgctgcccatgggagccctgccccaggggcccatgcaga
atgccaccaagtatggcaacatgacagaggaccatgtgatgcacctgctccagagtcactggaactggaggacccgtcttctggg
ctgggtgtga
Human invariant chain (hu-li, p33, clip) (PROTEIN)
(SEQ ID NO: 139)
MDDQRDLISNNEQLPMLGRRPGAPESKCSRGALYTGFSILVTLLLAGQATTAYFLYQQQGRL
DKLTVTSQNLQLENLRMKLPKPPKPVSKMRMATPLLMQALPMGALPQGPMQNATKYGNMT
EDHVMHLLQSHWNWRTRLLGWV*
Replace Clip sequence with sequence encoding for the Ag of interest:
huinsB 4-29 (DNA)
(SEQ ID NO: 140)
caacacctgtgcggctcacacctggtggaagctctctacctagtgtgcggggaacgaggcttcttctacacacccaag
huinsB 4-29 (protein) (epitope InsB₉₋₂₃)
(SEQ ID NO: 141)
QHLCGSHLVEALYLVCGERGFFYTPK
huinsB 4-29 (-14E -21E -22E) (DNA)
(SEQ ID NO: 142)
caacacctgtgcggctcacacctggtggaagaactctacctagtgtgcggggaagaaggcttcttctacacacccaag
huinsB 4-29 (-14E -21E -22E) (protein) (epitope InsB_{9-2314E-21E-22E})
(SEQ ID NO: 143)
QHLCGSHLVEELYLVCGEEGFFYTPK
hu.PPI Pre-pro insulin71-96 (DNA) (epitope PPI₇₁₋₉₆)
(SEQ ID NO: 144)
ggccctggtgcaggcagcctgcagcccttggccctggaggggtccctgcagaagcgtggcattgtggaacaatgctgt
hu.PPI (protein) (epitope C19A3)
(SEQ ID NO: 145)
GPGAGSLQPLALEGSLQKRGIVEQCC
hu.PPI Pre-pro insulin13-28(DNA) (PPI₁₃₋₂₈)
(SEQ ID NO: 146)
ctgctggccctctggggacctgacccagccgcagcctttgtgaaccaa
hu.PPI (protein) (epitope PPI₁₇₋₂₄)
(SEQ ID NO: 147)
LLALWGPDPAAAFVNQ
I-A2 801-821(DNA)
(SEQ ID NO: 148)
Gagagcggctgcaccgtcatcgtcatgctgaccccgctggtggaggatggtgtcaagcagtgt
I-A2(protein) (epitopes: I-A2 ₈₀₅₋₈₂₀; I-A2₈₀₆₋₈₁₄)
(SEQ ID NO: 149)
ESGCTVIVMLTPLVEDGVKQC
a2-gliadin 51-80>
(SEQ ID NO: 150)
tctcagcagccctacctgcaactgcagccctttccacagcctgagctgccctatcctcagcctcagcctagctttccacctcagcag
a2-gliadin 51-80>(protein) (epitope a2-g1i₅₅₋₇₆)
(SEQ ID NO: 151)
SQQPYLQLQPFPQPELPYPQPQPSFPPQQ
Tregitope289 (DNA)
(SEQ ID NO: 152)
Gaggagcagtacaacagcacctacagagtggtgagcgtgctgaccgtgctgcaccaggactgg
Tregitope289 (protein)
(SEQ ID NO: 153)
EEQYNSTYRVVSVLTVLHQDW
cloned into the following:
-Mono-directional LV backbones
LV.PGK.li
(SEQ ID NO: 154)
caggtggcacttttcggggaaatgtgcgcggaacccctatttgthatttttctaaatacattcaaatatgtatccgctcatgagacaataa
ccctgataaatgcttcaataatattgaaaaaggaagagtatgagtattcaacatttccgtgtcgcccttattccctiftttgcggcattttgcc
ttcctgtttttgctcacccagaaacgctggtgaaagtaaaagatgctgaagatcagttgggtgcacgagtgggttacatcgaactggat
ctcaacagcggtaagatccttgagagttttcgccccgaagaacgttttccaatgatgagcacttttaaagttctgctatgtggcgcggtat
tatcccgtattgacgccgggcaagagcaactcggtcgccgcatacactattctcagaatgacttggttgagtactcaccagtcacaga
aaagcatcttacggatggcatgacagtaagagaattatgcagtgctgccataaccatgagtgataacactgcggccaacttacttct
gacaacgatcggaggaccgaaggagctaaccgctiftttgcacaacatgggggatcatgtaactcgccttgatcgttgggaaccgg
agctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggccc
ttccggctggctggtttattgctgataaatctggagccggtgagcgtgggtctcgcggtatcattgcagcactggggccagatggtaag
ccctcccgtatcgtagttatctacacgacggggagtcaggcaactatggatgaacgaaatagacagatcgctgagataggtgcctc
actgattaagcattggtaactgtcagaccaagtttactcatatatactttagattgatttaaaacttcatttttaatttaaaaggatctaggtg
aagatcctifttgataatctcatgaccaaaatcccttaacgtgagttttcgttccactgagcgtcagaccccgtagaaaagatcaaagg
atcttcttgagatcctifttttctgcgcgtaatctgctgcttgcaaacaaaaaaaccaccgctaccagcggtggtttgtttgccggatcaag
agctaccaactctifttccgaaggtaactggcttcagcagagcgcagataccaaatactgtccttctagtgtagccgtagttaggccac
cacttcaagaactctgtagcaccgcctacatacctcgctctgctaatcctgttaccagtggctgctgccagtggcgataagtcgtgtctt
accgggttggactcaagacgatagttaccggataaggcgcagcggtcgggctgaacggggggttcgtgcacacagcccagcttg
gagcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttatagtc
ctgtcgggtttcgccacctctgacttgagcgtcgatttttgtgatgctcgtcaggggggcggagcctatggaaaaacgccagcaacgc
ggcctifttacggttcctggccttttgctggccttttgctcacatgttctttcctgcgttatcccctgattctgtggataaccgtattaccgcctttg
agtgagctgataccgctcgccgcagccgaacgaccgagcgcagcgagtcagtgagcgaggaagcggaagagcgcccaatac
gcaaaccgcctctccccgcgcgttggccgattcattaatgcagctggcacgacaggtttcccgactggaaagcgggcagtgagcg
caacgcaattaatgtgagttagctcactcattaggcaccccaggctttacactttatgcttccggctcgtatgttgtgtggaattgtgagcg
gataacaatttcacacaggaaacagctatgaccatgattacgccaagcgcgcaattaaccctcactaaagggaacaaaagctgg
agctgcaagcttggccattgcatacgttgtatccatatcataatatgtacatttatattggctcatgtccaacattaccgccatgttgacatt
gattattgactagttattaatagtaatcaattacggggtcattagttcatagcccatatatggagttccgcgttacataacttacggtaaat
ggcccgcctggctgaccgcccaacgacccccgcccattgacgtcaataatgacgtatgttcccatagtaacgccaatagggactttc
cattgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtattagt
catcgctattaccatggtgatgcggifttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatg
ggcggtaggcgtgtacggtgggaggtctatataagcagagctcgtttagtgaaccggggtctctctggttagaccagatctgagcctg
ggagctctctggctaactagggaacccactgcttaagcctcaataaagcttgccttgagtgcttcaagtagtgtgtgcccgtctgttgtgt
gactctggtaactagagatccctcagacccttttagtcagtgtggaaaatctctagcagtggcgcccgaacagggacctgaaagcg
aaagggaaaccagagctctctcgacgcaggactcggcttgctgaagcgcgcacggcaagaggcgaggggcggcgactggtga
gtacgccaaaaattttgactagcggaggctagaaggagagagatgggtgcgagagcgtcagtattaagcgggggagaattagat
cgcgatgggaaaaaattcggttaaggccagggggaaagaaaaaatataaattaaaacatatagtatgggcaagcagggagcta
gaacgattcgcagttaatcctggcctgttagaaacatcagaaggctgtagacaaatactgggacagctacaaccatcccttcagac
aggatcagaagaacttagatcattatataatacagtagcaaccctctattgtgtgcatcaaaggatagagataaaagacaccaagg
aagctttagacaagatagaggaagagcaaaacaaaagtaagaccaccgcacagcaagcggccgctgatcttcagacctggag
gaggagatatgagggacaattggagaagtgaattatataaatataaagtagtaaaaattgaaccattaggagtagcacccaccaa
ggcaaagagaagagtggtgcagagagaaaaaagagcagtgggaataggagctttgttccttgggttcttgggagcagcaggaa
gcactatgggcgcagcctcaatgacgctgacggtacaggccagacaattattgtctggtatagtgcagcagcagaacaatttgctga
gggctattgaggcgcaacagcatctgttgcaactcacagtctggggcatcaagcagctccaggcaagaatcctggctgtggaaag
atacctaaaggatcaacagctcctggggatttggggttgctctggaaaactcatttgcaccactgctgtgccttggaatgctagttgga
gtaataaatctctggaacagatttggaatcacacgacctggatggagtgggacagagaaattaacaattacacaagcttaatacact
ccttaattgaagaatcgcaaaaccagcaagaaaagaatgaacaagaattattggaattagataaatgggcaagtttgtggaattgg
tttaacataacaaattggctgtggtatataaaattattcataatgatagtaggaggcttggtaggtttaagaatagtifttgctgtactttctat
agtgaatagagttaggcagggatattcaccattatcgtttcagacccacctcccaaccccgaggggacccgacaggcccgaagga
atagaagaagaaggtggagagagagacagagacagatccattcgattagtgaacggatctcgacggtatcggttaacttttaaaa
gaaaaggggggattggggggtacagtgcaggggaaagaatagtagacataatagcaacagacatacaaactaaagaattaca
aaaacaaattacaaaaattcaaaattttatcgatcacgagactagcctcgagaagcttgatatcgaattcccacggggttggggttgc
gccttttccaaggcagccctgggtttgcgcagggacgcggctgctctgggcgtggttccgggaaacgcagcggcgccgaccctgq
gtctcgcacattcttcacgtccgttcgcagcgtcacccggatcttcgccgctacccttgtgggccccccggcgacgcttcctgctccgcc
cctaagtcgggaaggttccttgcggttcgcggcgtgccggacgtgacaaacggaagccgcacgtctcactagtaccctcgcagac
ggacagcgccagggagcaatggcagcgcgccgaccgcgatgggctgtggccaatagcggctgctcagcggggcgcgccgag
agcagcggccgggaaggggcggtgcgggaggcggggtgtggggcggtagtgtgggccctgttcctgcccgcgcggtgttccgca
ttctgcaagcctccggagcgcacgtcggcagtcggctccctcgttgaccgaatcaccgacctctctccccagggggatccaccatg
gatgaccaacgcgacctcatctctaaccatgaacagttgcccatactgggcaaccgccctagagagccagaaaggtgcagccgt
ggagctctgtacaccggtgtctctgtcctggtggctctgctcttggctgggcaggccaccactgcttacttcctgtaccagcaacaggg
ccgcctagacaagctgaccatcacctcccagaacctgcaactggagagccttcgcatgaagcttccgaaatctgccaaacctgtga
gccagatgcggatggctactcccttgctgatgcgtccaatgtccatggataacatgctccttgggcctgtgaagaacgttaccaagta
cggcaacatgacccaggaccatgtgatgcatctgctcacgaggtctggacccctggagtacccgcagctgaaggggaccttccca
gagaatctgaagcatcttaagaactccatggatggcgtgaactggaagatcttcgagagctggatgaagcagtggctcttgtttgag
atgagcaagaactccctggaggagaagaagcccaccgaggctccacctaaagagccactggacatggaagacctatcttctgg
cctgggagtgaccaggcaggaactgggtcaagtcaccctgtgtggcatctcctcagcagagagcctgaagatatctcaagctgtcc
atgcagcacatgcagaaatcaatgaagcaggcagagaggtggtagggtcagcagaggctggagtggatgctgcaagctgataa
gtcgacaatcaacctctggattacaaaatttgtgaaagattgactggtattcttaactatgttgctccttttacgctatgtggatacgctgctt
taatgcctttgtatcatgctattgcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggctgttggg
cactgacaattccgtggtgttgtcggggaagctgacgtcctttccatggctgctcgcctgtgttgccacctggattctgcgcgggacgtc
cttctgctacgtcccttcggccctcaatccagcggaccttccttcccgcggcctgctgccggctctgcggcctcttccgcgtcttcgccttc
gccctcagacgagtcggatctccctttgggccgcctccccgcctggaattcgagctcggtacctttaagaccaatgacttacaaggca
gctgtagatcttagccactifttaaaagaaaaggggggactggaagggctaattcactcccaacgaagacaagatctgctifttgcttg
tactgggtctctctggttagaccagatctgagcctgggagctctctggctaactagggaacccactgcttaagcctcaataaagcttgc
cttgagtgcttcaagtagtgtgtgcccgtctgttgtgtgactctggtaactagagatccctcagacccttttagtcagtgtggaaaatctcta
gcagtagtagttcatgtcatcttattattcagtatttataacttgcaaagaaatgaatatcagagagtgagaggaacttgtttattgcagctt
ataatggttacaaataaagcaatagcatcacaaatttcacaaataaagcattiftttcactgcattctagttgtggtttgtccaaactcatc
aatgtatcttatcatgtctggctctagctatcccgcccctaactccgcccagttccgcccattctccgccccatggctgactaattffitttatt
tatgcagaggccgaggccgcctcggcctctgagctattccagaagtagtgaggaggctiftttggaggcctaggcttttgcgtcgaga
cgtacccaattcgccctatagtgagtcgtattacgcgcgctcactggccgtcgttttacaacgtcgtgactgggaaaaccctggcgtta
cccaacttaatcgccttgcagcacatccccctttcgccagctggcgtaatagcgaagaggcccgcaccgatcgcccttcccaacagt
tgcgcagcctgaatggcgaatggcgcgacgcgccctgtagcggcgcattaagcgcggcgggtgtggtggttacgcgcagcgtga
ccgctacacttgccagcgccctagcgcccgctcctttcgctttcttcccttcctttctcgccacgttcgccggctttccccgtcaagctctaa
atcgggggctccctttagggttccgatttagtgctttacggcacctcgaccccaaaaaacttgattagggtgatggttcacgtagtgggc
catcgccctgatagacggffittcgccctttgacgttggagtccacgttctttaatagtggactcttgttccaaactggaacaacactcaac
cctatctcggtctattcttttgatttataagggattttgccgatttcggcctattggttaaaaaatgagctgatttaacaaaaatttaacgcga
attttaacaaaatattaacgtttacaatttcc
PGK.liAg.miR155T.miR146aT
(SEQ ID NO: 155)
caggtggcacttttcggggaaatgtgcgcggaacccctatttgtttatttttctaaatacattcaaatatgtatccgctcatgagacaataa
ccctgataaatgcttcaataatattgaaaaaggaagagtatgagtattcaacatttccgtgtcgcccttattccctiftttgcggcattttgcc
ttcctgffittgctcacccagaaacgctggtgaaagtaaaagatgctgaagatcagttgggtgcacgagtgggttacatcgaactggat
ctcaacagcggtaagatccttgagagttttcgccccgaagaacgttttccaatgatgagcacttttaaagttctgctatgtggcgcggtat
tatcccgtattgacgccgggcaagagcaactcggtcgccgcatacactattctcagaatgacttggttgagtactcaccagtcacaga
aaagcatcttacggatggcatgacagtaagagaattatgcagtgctgccataaccatgagtgataacactgcggccaacttacttct
gacaacgatcggaggaccgaaggagctaaccgctiftttgcacaacatgggggatcatgtaactcgccttgatcgttgggaaccgg
agctgaatgaagccataccaaacgacgagcgtgacaccacgatgcctgtagcaatggcaacaacgttgcgcaaactattaactg
gcgaactacttactctagcttcccggcaacaattaatagactggatggaggcggataaagttgcaggaccacttctgcgctcggccc
ttccggctggctggtttattgctgataaatctggagccggtgagcgtgggtctcgcggtatcattgcagcactggggccagatggtaag
ccctcccgtatcgtagttatctacacgacggggagtcaggcaactatggatgaacgaaatagacagatcgctgagataggtgcctc
actgattaagcattggtaactgtcagaccaagtttactcatatatactttagattgatttaaaacttcatttttaatttaaaaggatctaggtg
aagatcctifttgataatctcatgaccaaaatcccttaacgtgagttttcgttccactgagcgtcagaccccgtagaaaagatcaaagg
atcttcttgagatcctifttttctgcgcgtaatctgctgcttgcaaacaaaaaaaccaccgctaccagcggtggtttgtttgccggatcaag
agctaccaactctifttccgaaggtaactggcttcagcagagcgcagataccaaatactgtccttctagtgtagccgtagttaggccac
cacttcaagaactctgtagcaccgcctacatacctcgctctgctaatcctgttaccagtggctgctgccagtggcgataagtcgtgtctt
accgggttggactcaagacgatagttaccggataaggcgcagcggtcgggctgaacggggggttcgtgcacacagcccagcttg
gagcgaacgacctacaccgaactgagatacctacagcgtgagctatgagaaagcgccacgcttcccgaagggagaaaggcgg
acaggtatccggtaagcggcagggtcggaacaggagagcgcacgagggagcttccagggggaaacgcctggtatctttatagtc
ctgtcgggtttcgccacctctgacttgagcgtcgatttttgtgatgctcgtcaggggggcggagcctatggaaaaacgccagcaacgc
ggcctifttacggttcctggccttttgctggccttttgctcacatgttctttcctgcgttatcccctgattctgtggataaccgtattaccgcctttg
agtgagctgataccgctcgccgcagccgaacgaccgagcgcagcgagtcagtgagcgaggaagcggaagagcgcccaatac
gcaaaccgcctctccccgcgcgttggccgattcattaatgcagctggcacgacaggtttcccgactggaaagcgggcagtgagcg
caacgcaattaatgtgagttagctcactcattaggcaccccaggctttacactttatgcttccggctcgtatgttgtgtggaattgtgagcg
gataacaatttcacacaggaaacagctatgaccatgattacgccaagcgcgcaattaaccctcactaaagggaacaaaagctgg
agctgcaagcttggccattgcatacgttgtatccatatcataatatgtacatttatattggctcatgtccaacattaccgccatgttgacatt
gattattgactagttattaatagtaatcaattacggggtcattagttcatagcccatatatggagttccgcgttacataacttacggtaaat
ggcccgcctggctgaccgcccaacgacccccgcccattgacgtcaataatgacgtatgttcccatagtaacgccaatagggactttc
cattgacgtcaatgggtggagtatttacggtaaactgcccacttggcagtacatcaagtgtatcatatgccaagtacgccccctattga
cgtcaatgacggtaaatggcccgcctggcattatgcccagtacatgaccttatgggactttcctacttggcagtacatctacgtattagt
catcgctattaccatggtgatgcggifttggcagtacatcaatgggcgtggatagcggtttgactcacggggatttccaagtctccaccc
cattgacgtcaatgggagtttgttttggcaccaaaatcaacgggactttccaaaatgtcgtaacaactccgccccattgacgcaaatg
ggcggtaggcgtgtacggtgggaggtctatataagcagagctcgtttagtgaaccggggtctctctggttagaccagatctgagcctg
ggagctctctggctaactagggaacccactgcttaagcctcaataaagcttgccttgagtgcttcaagtagtgtgtgcccgtctgttgtgt
gactctggtaactagagatccctcagacccttttagtcagtgtggaaaatctctagcagtggcgcccgaacagggacctgaaagcg
aaagggaaaccagagctctctcgacgcaggactcggcttgctgaagcgcgcacggcaagaggcgaggggcggcgactggtga
gtacgccaaaaattttgactagcggaggctagaaggagagagatgggtgcgagagcgtcagtattaagcgggggagaattagat
cgcgatgggaaaaaattcggttaaggccagggggaaagaaaaaatataaattaaaacatatagtatgggcaagcagggagcta
gaacgattcgcagttaatcctggcctgttagaaacatcagaaggctgtagacaaatactgggacagctacaaccatcccttcagac
aggatcagaagaacttagatcattatataatacagtagcaaccctctattgtgtgcatcaaaggatagagataaaagacaccaagg
aagctttagacaagatagaggaagagcaaaacaaaagtaagaccaccgcacagcaagcggccgctgatcttcagacctggag
gaggagatatgagggacaattggagaagtgaattatataaatataaagtagtaaaaattgaaccattaggagtagcacccaccaa
ggcaaagagaagagtggtgcagagagaaaaaagagcagtgggaataggagctttgttccttgggttcttgggagcagcaggaa
gcactatgggcgcagcctcaatgacgctgacggtacaggccagacaattattgtctggtatagtgcagcagcagaacaatttgctga
gggctattgaggcgcaacagcatctgttgcaactcacagtctggggcatcaagcagctccaggcaagaatcctggctgtggaaag
atacctaaaggatcaacagctcctggggatttggggttgctctggaaaactcatttgcaccactgctgtgccttggaatgctagttgga
gtaataaatctctggaacagatttggaatcacacgacctggatggagtgggacagagaaattaacaattacacaagcttaatacact
ccttaattgaagaatcgcaaaaccagcaagaaaagaatgaacaagaattattggaattagataaatgggcaagtttgtggaattgg
tttaacataacaaattggctgtggtatataaaattattcataatgatagtaggaggcttggtaggtttaagaatagthttgctgtactttctat
agtgaatagagttaggcagggatattcaccattatcgtttcagacccacctcccaaccccgaggggacccgacaggcccgaagga
atagaagaagaaggtggagagagagacagagacagatccattcgattagtgaacggatctcgacggtatcggttaacttttaaaa
gaaaaggggggattggggggtacagtgcaggggaaagaatagtagacataatagcaacagacatacaaactaaagaattaca
aaaacaaattacaaaaattcaaaattttatcgatcacgagactagcctcgagaagcttgatatcgaattcccacggggttggggttgc
gccttttccaaggcagccctgggtttgcgcagggacgcggctgctctgggcgtggttccgggaaacgcagcggcgccgaccctgo
gtctcgcacattcttcacgtccgttcgcagcgtcacccggatcttcgccgctacccttgtgggccccccggcgacgcttcctgctccgcc
cctaagtcgggaaggttccttgcggttcgcggcgtgccggacgtgacaaacggaagccgcacgtctcactagtaccctcgcagac
ggacagcgccagggagcaatggcagcgcgccgaccgcgatgggctgtggccaatagcggctgctcagcggggcgcgccgag
agcagcggccgggaaggggcggtgcgggaggcggggtgtggggcggtagtgtgggccctgttcctgcccgcgcggtgttccgca
ttctgcaagcctccggagcgcacgtcggcagtcggctccctcgttgaccgaatcaccgacctctctccccagggggatccaccatg
gatgaccaacgcgacctcatctctaaccatgaacagttgcccatactgggcaaccgccctagagagccagaaaggtgcagccgt
ggagctctgtacaccggtgtctctgtcctggtggctctgctcttggctgggcaggccaccactgcttacttcctgtaccagcaacaggg
ccgcctagacaagctgaccatcacctcccagaacctgcaactggagagccttcgcatgaagcttccgaaatctgccaaacctgtga
gccagatgcggatggctactcccttgctgatgcgtccaatgtccatggataacatgctccttgggcctgtgaagaacgttaccaagta
cggcaacatgacccaggaccatgtgatgcatctgctcacgaggtctggacccctggagtacccgcagctgaaggggaccttccca
gagaatctgaagcatcttaagaactccatggatggcgtgaactggaagatcttcgagagctggatgaagcagtggctcttgtttgag
atgagcaagaactccctggaggagaagaagcccaccgaggctccacctaaagagccactggacatggaagacctatcttctgg
cctgggagtgaccaggcaggaactgggtcaagtcaccctgtgtggcatctcctcagcagagagcctgaagatatctcaagctgtcc
atgcagcacatgcagaaatcaatgaagcaggcagagaggtggtagggtcagcagaggctggagtggatgctgcaagctgataa
gtcgacaatcaacctctggattacaaaatttgtgaaagattgactggtattcttaactatgttgctccttttacgctatgtggatacgctgctt
taatgcctttgtatcatgctattgcttcccgtatggctttcattttctcctccttgtataaatcctggttgctgtctctttatgaggagttgtggccc
gttgtcaggcaacgtggcgtggtgtgcactgtgtttgctgacgcaacccccactggttggggcattgccaccacctgtcagctcctttcc
gggactttcgctttccccctccctattgccacggcggaactcatcgccgcctgccttgcccgctgctggacaggggctcggctgttggg
cactgacaattccgtggtgttgtcggggaagctgacgtcctttccatggctgctcgcctgtgttgccacctggattctgcgcgggacgtc
cttctgctacgtcccttcggccctcaatccagcggaccttccttcccgcggcctgctgccggctctgcggcctcttccgcgtcttcgccttc
gccctcagacgagtcggatctccctttgggccgcctccccgcctggaattcgagctcgctagctaacccctatcacaattagcattaa
cgatcccctatcacaattagcattaaaccggtcccctatcacaattagcattaatcaccccctatcacaattagcattaacccggggta
aaacccatggaattcagttctcacgataacccatggaattcagttctcaacgcgtaacccatggaattcagttctcatcacaa
cccatggaattcagttctcaggtacctttaagaccaatgacttacaaggcagctgtagatcttagccactifttaaaagaaaagggg
ggactggaagggctaattcactcccaacgaagacaagatctgctifttgcttgtactgggtctctctggttagaccagatctgagcctgg
gagctctctggctaactagggaacccactgcttaagcctcaataaagcttgccttgagtgcttcaagtagtgtgtgcccgtctgttgtgtg
actctggtaactagagatccctcagacccttttagtcagtgtggaaaatctctagcagtagtagttcatgtcatcttattattcagtatttata
acttgcaaagaaatgaatatcagagagtgagaggaacttgtttattgcagcttataatggttacaaataaagcaatagcatcacaaat
ttcacaaataaagcattiftttcactgcattctagttgtggtttgtccaaactcatcaatgtatcttatcatgtctggctctagctatcccgcccc
taactccgcccagttccgcccattctccgccccatggctgactaattffitttatttatgcagaggccgaggccgcctcggcctctgagct
attccagaagtagtgaggaggctffittggaggcctaggcttttgcgtcgagacgtacccaattcgccctatagtgagtcgtattacgcg
cgctcactggccgtcgttttacaacgtcgtgactgggaaaaccctggcgttacccaacttaatcgccttgcagcacatccccctttcgc
cagctggcgtaatagcgaagaggcccgcaccgatcgcccttcccaacagttgcgcagcctgaatggcgaatggcgcgacgcgc
cctgtagcggcgcattaagcgcggcgggtgtggtggttacgcgcagcgtgaccgctacacttgccagcgccctagcgcccgctcctt
tcgctttcttcccttcctttctcgccacgttcgccggctttccccgtcaagctctaaatcgggggctccctttagggttccgatttagtgcttta
cggcacctcgaccccaaaaaacttgattagggtgatggttcacgtagtgggccatcgccctgatagacggiftttcgccctttgacgtt
ggagtccacgttctttaatagtggactcttgttccaaactggaacaacactcaaccctatctcggtctattcttttgatttataagggattttg
ccgatttcggcctattggttaaaaaatgagctgatttaacaaaaatttaacgcgaattttaacaaaatattaacgtttacaatttcc
Bi-directional LV backbones
bd.ΔNGFR.PGK.GFP
(SEQ ID NO: 156)
aaatttcacaaataaagcattiftttcactgcattctagttgtggtttgtccaaactcatcaatgtatcttatcatgtctggctctagctatcccg
cccctaactccgcccagttccgcccattctccgccccatggctgactaattifttttatttatgcagaggccgaggccgcctcggcctctg
agctattccagaagtagtgaggaggctffittggaggcctaggcttttgcgtcgagacgtacccaattcgccctatagtgagtcgtatta
cgcgcgctcactggccgtcgttttacaacgtcgtgactgggaaaaccctggcgttacccaacttaatcgccttgcagcacatccccctt
tcgccagctggcgtaatagcgaagaggcccgcaccgatcgcccttcccaacagttgcgcagcctgaatggcgaatggcgcgacg
cgccctgtagcggcgcattaagcgcggcgggtgtggtggttacgcgcagcgtgaccgctacacttgccagcgccctagcgcccgct
cctttcgctttcttcccttcctttctcgccacgttcgccggctttccccgtcaagctctaaatcgggggctccctttagggttccgatttagtgc
tttacggcacctcgaccccaaaaaacttgattagggtgatggttcacgtagtgggccatcgccctgatagacggiftttcgccctttgac
gttggagtccacgttctttaatagtggactcttgttccaaactggaacaacactcaaccctatctcggtctattcttttgatttataagggattt
tgccgatttcggcctattggttaaaaaatgagctgatttaacaaaaatttaacgcgaattttaacaaaatattaacgtttacaatttcccag
gtggcacttttcggggaaatgtgcgcggaacccctatttgtttatttttctaaatacattcaaatatgtatccgctcatgagacaataaccct
gataaatgcttcaataatattgaaaaaggaagagtatgagtattcaacatttccgtgtcgcccttattccctiftttgcggcattttgccttcc
tgtttttgctcacccagaaacgctggtgaaagtaaaagatgctgaagatcagttgggtgcacgagtgggttacatcgaactggatctc
aacagcggtaagatccttgagagttttcgccccgaagaacgttttccaatgatgagcacttttaaagttctgctatgtggcgcggtattat
cccgtattgacgccgggcaagagcaactcggtcgccgcatacactattctcagaatgacttggttgagtactcaccagtcacagaaa
agcatcttacggatggcatgacagtaagagaattatgcagtgctgccataaccatgagtgataacactgcggccaacttacttctgac
aacgatcggaggaccgaaggagctaaccgctffittgcacaacatgggggatcatgtaactcgccttgatcgttgggaaccggagct
gaatgaagccataccaaacgacgagcgtgacaccacgatgcctgtagcaatggcaacaacgttgcgcaaactattaactggcga
actacttactctagcttcccggcaacaattaatagactggatggaggcggataaagttgcaggaccacttctgcgctcggcccttccg
gctggctggtttattgctgataaatctggagccggtgagcgtgggtctcgcggtatcattgcagcactggggccagatggtaagccctc
ccgtatcgtagttatctacacgacggggagtcaggcaactatggatgaacgaaatagacagatcgctgagataggtgcctcactga
ttaagcattggtaactgtcagaccaagtttactcatatatactttagattgatttaaaacttcatttttaatttaaaaggatctaggtgaagat
cctifttgataatctcatgaccaaaatcccttaacgtgagttttcgttccactgagcgtcagaccccgtagaaaagatcaaaggatcttct
tgagatcctifttttctgcgcgtaatctgctgcttgcaaacaaaaaaaccaccgctaccagcggtggtttgtttgccggatcaagagcta
ccaactctifttccgaaggtaactggcttcagcagagcgcagataccaaatactgtccttctagtgtagccgtagttaggccaccacttc
aagaactctgtagcaccgcctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacagg
tatccggtaagcggcagggtcggaacaggagagcgcacgagggagcttccagggggaaacgcctggtatctttatagtcctgtcg
ggtttcgccacctctgacttgagcgtcgatttttgtgatgctcgtcaggggggcggagcctatggaaaaacgccagcaacgcggcctt
tttacggttcctggccttttgctggccttttgctcacatgttctttcctgcgttatcccctgattctgtggataaccgtattaccgcctttgagtga
gctgataccgctcgccgcagccgaacgaccgagcgcagcgagtcagtgagcgaggaagcggaagagcgcccaatacgcaaa
ccgcctctccccgcgcgttggccgattcattaatgcagctggcacgacaggtttcccgactggaaagcgggcagtgagcgcaacg
caattaatgtgagttagctcactcattaggcaccccaggctttacactttatgcttccggctcgtatgttgtgtggaattgtgagcggataa
caatttcacacaggaaacagctatgaccatgattacgccaagcgcgcaattaaccctcactaaagggaacaaaagctggagctg
caagcttggccattgcatacgttgtatccatatcataatatgtacatttatattggctcatgtccaacattaccgccatgttgacattgattatt
gactagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgac
gtcaatgggtggagtatttacggtaaactgcccacttggcagtacatcaagtgtatcatatgccaagtacgccccctattgacgtcaat
gacggtaaatggcccgcctggcattatgcccagtacatgaccttatgggactttcctacttggcagtacatctacgtattagtcatcgct
attaccatggtgatgcggifttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgcaaatgggcggta
ggcgtgtacggtgggaggtctatataagcagagctcgtttagtgaaccggggtctctctggttagaccagatctgagcctgggagctc
tctggctaactagggaacccactgcttaagcctcaataaagcttgccttgagtgcttcaagtagtgtgtgcccgtctgttgtgtgactctg
gtaactagagatccctcagacccttttagtcagtgtggaaaatctctagcagtggcgcccgaacagggacctgaaagcgaaaggg
aaaccagagctctctcgacgcaggactcggcttgctgaagcgcgcacggcaagaggcgaggggcggcgactggtgagtacgc
caaaaattttgactagcggaggctagaaggagagagatgggtgcgagagcgtcagtattaagcgggggagaattagatcgcgat
gggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttt
agacaagatagaggaagagcaaaacaaaagtaagaccaccgcacagcaagcggccgctgatcttcagacctggaggagga
gatatgagggacaattggagaagtgaattatataaatataaagtagtaaaaattgaaccattaggagtagcacccaccaaggcaa
agagaagagtggtgcagagagaaaaaagagcagtgggaataggagctttgttccttgggttcttgggagcagcaggaagcacta
tgggcgcagcctcaatgacgctgacggtacaggccagacaattattgtctggtatagtgcagcagcagaacaatttgctgagggct
attgaggcgcaacagcatctgttgcaactcacagtctggggcatcaagcagctccaggcaagaatcctggctgtggaaagatacct
aaaggatcaacagctcctggggatttggggttgctctggaaaactcatttgcaccactgctgtgccttggaatgctagttggagtaata
aatctctggaacagattggaatcacacgacctggatggagtgggacagagaaattaacaattacacaagcttaatacactccttaat
tgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtifttgctgtactttctatagtgaa
tagagttaggcagggatattcaccattatcgtttcagacccacctcccaaccccgaggggacccgacaggcccgaaggaatagaa
gaagaaggtggagagagagacagagacagatccattcgattagtgaacggatctcgacggtatcggttaacttttaaaagaaaag
gggggattggggggtacagtgcaggggaaagaatagtagacataatagcaacagacatacaaactaaagaattacaaaaaca
aattacaaaaattcaaaattttatcgatcacgagactagcctcgagagatctgatcataatcagccataccacatttgtagaggifttac
ttgctttaaaaaacctcccacacctccccctgaacctgaaacataaaatgaatgcaattgttgttgttaacttgtttattgcagcttataatg
gttacaaataaggcaatagcatcacaaatttcacaaataaggcattffittcactgcattctagttttggtttgtccaaactcatcaatgtat
cttatcatgtctggatctcaaatccctcggaagctgcgcctgtcatcgaattcctgcagcccggtgcatgactaagctagctcagttagc
ctcccccatctcccctagaggatccccctgttccacctcttgaaggctatgtaggccacaaggcccacaaccacagcagcc
aggatggagcaatagacagggatgaggttgtcggtggtgcctcgggtcaccacgggctgggagctgcccatcactgtg
gtcaccacacctgccaccgtgctggctatgaggtcttgttctggaggtgcctcaggctcctgggtgctgggggctgtgctg
tccgagccctctgggggtgtggaccgtgtaatccaacggccagggatctcctcgcactcggcgtcggcccagcgtgtgcactc
gcggagctggcgctcggtgtcctcgcacacggtgcagggcaggcacgggtccacgtggttggcctcgtcggaatacgtgccgtcg
gggcactcctcgcacacggtgttctgcttgtcctggcaggagaacacgaggcccgagcccgcctcgcacacgcggcacgcctcg
cagcgcccagtcgtctcatcctggtagtagccgtaggcgcagcggcacacggcgtcgtcggcctccacgcacggcgccgacatg
ctctggagccccacgcactcggtgcacggcttgcacggctcggtcgcgctcaccacgtcggagaacgtcacgctgtccaggcagg
gctcacacacggtctggttggctccacaaggctgggccacaccctcgcccaggttgcaggctttgcagcactcaccgctgtgtgtgta
caggcctgtggggcatgcctccttggcacctccaagggacacccccagaagcagcaacagcagcaggcgcggcccgtccatgg
cgcggccggtggcacctgcccccatcgcccgcctcccgcggcagcgctcgacttccagctcggtccgctttgcggactgatggggc
tgcgctgcgctgcgctccagcgccccccctgcccgccggagctggccgcggcccgaattccgcggaggctggatcggtcccggtg
tcttctatggaggtcaaaacagcgtggatggcgtctccaggcgatctgacggttcactaaacgagctctgcttatataggcctcccacc
gtacacgcctaccctcgagaagcttgatatcgaattcccacggggttggggttgcgccttttccaaggcagccctgggtttgcgcagg
gacgcggctgctctgggcgtggttccgggaaacgcagcggcgccgaccctgggtctcgcacattcttcacgtccgttcgcagcgtca
cccggatcttcgccgctacccttgtgggccccccggcgacgcttcctgctccgcccctaagtcgggaaggttccttgcggttcgcggc
gtgccggacgtgacaaacggaagccgcacgtctcactagtaccctcgcagacggacagcgccagggagcaatggcagcgcgc
cgaccgcgatgggctgtggccaatagcggctgctcagcggggcgcgccgagagcagcggccgggaaggggcggtgcgggag
gcggggtgtggggcggtagtgtgggccctgttcctgcccgcgcggtgttccgcattctgcaagcctccggagcgcacgtcggcagtc
ggctccctcgttgaccgaatcaccgacctctctccccagggggatccaccggtcgccaccatggtgagcaagggcgaggagctgtt
caccggggtggtgcccatcctggtcgagctggacggcgacgtaaacggccacaagttcagcgtgtccggcgagggcgagggcg
atgccacctacggcaagctgaccctgaagttcatctgcaccaccggcaagctgcccgtgccctggcccaccctcgtgaccaccctg
acctacggcgtgcagtgcttcagccgctaccccgaccacatgaagcagcacgacttcttcaagtccgccatgcccgaaggctacgt
ccaggagcgcaccatcttcttcaaggacgacggcaactacaagacccgcgccgaggtgaagttcgagggcgacaccctggtga
accgcatcgagctgaagggcatcgacttcaaggaggacggcaacatcctggggcacaagctggagtacaactacaacagcca
caacgtctatatcatggccgacaagcagaagaacggcatcaaggtgaacttcaagatccgccacaacatcgaggacggcagcg
tgcagctcgccgaccactaccagcagaacacccccatcggcgacggccccgtgctgctgcccgacaaccactacctgagcacc
cagtccgccctgagcaaagaccccaacgagaagcgcgatcacatggtcctgctggagttcgtgaccgccgccgggatcactctcg
gcatggacgagctgtacaagtaaagcggccgcgtcgacaatcaacctctggattacaaaatttgtgaaagattgactggtattcttaa
ctatgttgctccttttacgctatgtggatacgctgctttaatgcctttgtatcatgctattgcttcccgtatggctttcattttctcctccttgtataa
atcctggttgctgtctctttatgaggagttgtggcccgttgtcaggcaacgtggcgtggtgtgcactgtgtttgctgacgcaacccccact
ggttggggcattgccaccacctgtcagctcctttccgggactttcgctttccccctccctattgccacggcggaactcatcgccgcctgc
cttgcccgctgctggacaggggctcggctgttgggcactgacaattccgtggtgttgtcggggaagctgacgtcctttccatggctgctc
gcctgtgttgccacctggattctgcgcgggacgtccttctgctacgtcccttcggccctcaatccagcggaccttccttcccgcggcctg
ctgccggctctagagcctcttccgcgtcttcgccttcccgggtcgagctcggtacctttaagaccaatgacttacaaggcagctgtaga
tcttagccactifttaaaagaaaaggggggactggaagggctaattcactcccaacgaagacaagatctgctifttgcttgtactgggt
ctctctggttagaccagatctgagcctgggagctctctggctaactagggaacccactgcttaagcctcaataaagcttgccttgagtg
cttcaagtagtgtgtgcccgtctgttgtgtgactctggtaactagagatccctcagacccttttagtcagtgtggaaaatctctagcagtag
tagttcatgtcatcttattattcagtatttataacttgcaaagaaatgaatatcagagagtgagaggaacttgthattgcagcttataatggt
tacaaataaagcaatagcatcac
bd.IL10.PGK.GFP
(SEQ ID NO: 157)
aaatttcacaaataaagcattiftttcactgcattctagttgtggtttgtccaaactcatcaatgtatcttatcatgtctggctctagctatcccg
cccctaactccgcccagttccgcccattctccgccccatggctgactaattifttttatttatgcagaggccgaggccgcctcggcctctg
agctattccagaagtagtgaggaggctiftttggaggcctaggcttttgcgtcgagacgtacccaattcgccctatagtgagtcgtatta
cgcgcgctcactggccgtcgttttacaacgtcgtgactgggaaaaccctggcgttacccaacttaatcgccttgcagcacatccccctt
tcgccagctggcgtaatagcgaagaggcccgcaccgatcgcccttcccaacagttgcgcagcctgaatggcgaatggcgcgacg
cgccctgtagcggcgcattaagcgcggcgggtgtggtggttacgcgcagcgtgaccgctacacttgccagcgccctagcgcccgct
cctttcgctttcttcccttcctttctcgccacgttcgccggctttccccgtcaagctctaaatcgggggctccctttagggttccgatttagtgc
tttacggcacctcgaccccaaaaaacttgattagggtgatggttcacgtagtgggccatcgccctgatagacggthttcgccctttgac
gttggagtccacgttctttaatagtggactcttgttccaaactggaacaacactcaaccctatctcggtctattcttttgatttataagggattt
tgccgatttcggcctattggttaaaaaatgagctgatttaacaaaaatttaacgcgaattttaacaaaatattaacgtttacaatttcccag
gtggcacttttcggggaaatgtgcgcggaacccctatttgtttatttttctaaatacattcaaatatgtatccgctcatgagacaataaccct
gataaatgcttcaataatattgaaaaaggaagagtatgagtattcaacatttccgtgtcgcccttattccctiftttgcggcattttgccttcc
tgtttttgctcacccagaaacgctggtgaaagtaaaagatgctgaagatcagttgggtgcacgagtgggttacatcgaactggatctc
aacagcggtaagatccttgagagttttcgccccgaagaacgttttccaatgatgagcacttttaaagttctgctatgtggcgcggtattat
cccgtattgacgccgggcaagagcaactcggtcgccgcatacactattctcagaatgacttggttgagtactcaccagtcacagaaa
agcatcttacggatggcatgacagtaagagaattatgcagtgctgccataaccatgagtgataacactgcggccaacttacttctgac
aacgatcggaggaccgaaggagctaaccgctffittgcacaacatgggggatcatgtaactcgccttgatcgttgggaaccggagct
gaatgaagccataccaaacgacgagcgtgacaccacgatgcctgtagcaatggcaacaacgttgcgcaaactattaactggcga
actacttactctagcttcccggcaacaattaatagactggatggaggcggataaagttgcaggaccacttctgcgctcggcccttccg
gctggctggtttattgctgataaatctggagccggtgagcgtgggtctcgcggtatcattgcagcactggggccagatggtaagccctc
ccgtatcgtagttatctacacgacggggagtcaggcaactatggatgaacgaaatagacagatcgctgagataggtgcctcactga
ttaagcattggtaactgtcagaccaagtttactcatatatactttagattgatttaaaacttcatttttaatttaaaaggatctaggtgaagat
cctifttgataatctcatgaccaaaatcccttaacgtgagttttcgttccactgagcgtcagaccccgtagaaaagatcaaaggatcttct
tgagatcctifttttctgcgcgtaatctgctgcttgcaaacaaaaaaaccaccgctaccagcggtggtttgtttgccggatcaagagcta
ccaactctifttccgaaggtaactggcttcagcagagcgcagataccaaatactgtccttctagtgtagccgtagttaggccaccacttc
aagaactctgtagcaccgcctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacagg
tatccggtaagcggcagggtcggaacaggagagcgcacgagggagcttccagggggaaacgcctggtatctttatagtcctgtcg
ggtttcgccacctctgacttgagcgtcgatttttgtgatgctcgtcaggggggcggagcctatggaaaaacgccagcaacgcggcctt
tttacggttcctggccttttgctggccttttgctcacatgttctttcctgcgttatcccctgattctgtggataaccgtattaccgcctttgagtga
gctgataccgctcgccgcagccgaacgaccgagcgcagcgagtcagtgagcgaggaagcggaagagcgcccaatacgcaaa
ccgcctctccccgcgcgttggccgattcattaatgcagctggcacgacaggtttcccgactggaaagcgggcagtgagcgcaacg
caattaatgtgagttagctcactcattaggcaccccaggctttacactttatgcttccggctcgtatgttgtgtggaattgtgagcggataa
caatttcacacaggaaacagctatgaccatgattacgccaagcgcgcaattaaccctcactaaagggaacaaaagctggagctg
caagcttggccattgcatacgttgtatccatatcataatatgtacatttatattggctcatgtccaacattaccgccatgttgacattgattatt
gactagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgac
gtcaatgggtggagtatttacggtaaactgcccacttggcagtacatcaagtgtatcatatgccaagtacgccccctattgacgtcaat
gacggtaaatggcccgcctggcattatgcccagtacatgaccttatgggactttcctacttggcagtacatctacgtattagtcatcgct
attaccatggtgatgcggifttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgcaaatgggcggta
ggcgtgtacggtgggaggtctatataagcagagctcgtttagtgaaccggggtctctctggttagaccagatctgagcctgggagctc
tctggctaactagggaacccactgcttaagcctcaataaagcttgccttgagtgcttcaagtagtgtgtgcccgtctgttgtgtgactctg
gtaactagagatccctcagacccttttagtcagtgtggaaaatctctagcagtggcgcccgaacagggacctgaaagcgaaaggg
aaaccagagctctctcgacgcaggactcggcttgctgaagcgcgcacggcaagaggcgaggggcggcgactggtgagtacgc
caaaaattttgactagcggaggctagaaggagagagatgggtgcgagagcgtcagtattaagcgggggagaattagatcgcgat
gggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttt
agacaagatagaggaagagcaaaacaaaagtaagaccaccgcacagcaagcggccgctgatcttcagacctggaggagga
gatatgagggacaattggagaagtgaattatataaatataaagtagtaaaaattgaaccattaggagtagcacccaccaaggcaa
agagaagagtggtgcagagagaaaaaagagcagtgggaataggagctttgttccttgggttcttgggagcagcaggaagcacta
tgggcgcagcctcaatgacgctgacggtacaggccagacaattattgtctggtatagtgcagcagcagaacaatttgctgagggct
attgaggcgcaacagcatctgttgcaactcacagtctggggcatcaagcagctccaggcaagaatcctggctgtggaaagatacct
aaaggatcaacagctcctggggatttggggttgctctggaaaactcatttgcaccactgctgtgccttggaatgctagttggagtaata
aatctctggaacagattggaatcacacgacctggatggagtgggacagagaaattaacaattacacaagcttaatacactccttaat
tgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagtifttgctgtactttctatagtgaa
tagagttaggcagggatattcaccattatcgtttcagacccacctcccaaccccgaggggacccgacaggcccgaaggaatagaa
gaagaaggtggagagagagacagagacagatccattcgattagtgaacggatctcgacggtatcggttaacttttaaaagaaaag
gggggattggggggtacagtgcaggggaaagaatagtagacataatagcaacagacatacaaactaaagaattacaaaaaca
aattacaaaaattcaaaattttatcgatcacgagactagcctcgagagatctgatcataatcagccataccacatttgtagaggifttac
ttgctttaaaaaacctcccacacctccccctgaacctgaaacataaaatgaatgcaattgttgttgttaacttgtttattgcagcttataatg
gttacaaataaggcaatagcatcacaaatttcacaaataaggcattiftttcactgcattctagttttggthgtccaaactcatcaatgtat
cttatcatgtctggatctcaaatccctcggaagctgcgcctgtcatcgaattcctgcagcccggtgcatgactaagctagcagttcagt
tccggatcttcatggtcatgtaggcctcgatgtagttgatgaagatgtcgaactcgctcatggccttgtagatgcccttttcct
gcagcttgttgaaggcgtttttgacctgttccacggccttgctcttgttctcgcagggcagaaatctgtggcaccgcctcag
ccgcagccgcagggttttcaggttctcgcccaggctgttcacgtgggccttgatgtcggggtcctggttctcggcctgggg
catcacttcttccaggtagaactggatcatctcgctcagggcctggcagcccaggtagcccttgaaatcttccagcaggctctctttca
gcagcaggttgtccagctggtccttcatctggaagaatgttttcactctgctgaaggcgtccctcaggtcccgcagcatgttgggcagg
ttgccggggaagtgggtgcagctgttctcgctctgggtgccctggccaggagaggctctgacgccggtcagcagcaccaggcagc
acagcagggcggagctgtgcatagtcggtccgctttgcggactgatggggctgcgctgcgctgcgctccagcgccccccctgcccg
ccggagctggccgcggcccgaattccgcggaggctggatcggtcccggtgtcttctatggaggtcaaaacagcgtggatggcgtct
ccaggcgatctgacggttcactaaacgagctctgcttatataggcctcccaccgtacacgcctaccctcgagaagcttgatatcgaat
tcccacggggttggggttgcgccttttccaaggcagccctgggtttgcgcagggacgcggctgctctgggcgtggttccgggaaacg
cagcggcgccgaccctgggtctcgcacattcttcacgtccgttcgcagcgtcacccggatcttcgccgctacccttgtgggccccccg
gcgacgcttcctgctccgcccctaagtcgggaaggttccttgcggttcgcggcgtgccggacgtgacaaacggaagccgcacgtct
cactagtaccctcgcagacggacagcgccagggagcaatggcagcgcgccgaccgcgatgggctgtggccaatagcggctgct
cagcggggcgcgccgagagcagcggccgggaaggggcggtgcgggaggcggggtgtggggcggtagtgtgggccctgttcct
gcccgcgcggtgttccgcattctgcaagcctccggagcgcacgtcggcagtcggctccctcgttgaccgaatcaccgacctctctcc
ccagggggatccaccatggatgaccaacgcgacctcatctctaaccatgaacagttgcccatactgggcaaccgccctagagag
ccagaaaggtgcagccgtggagctctgtacaccggtgtctctgtcctggtggctctgctcttggctgggcaggccaccactgcttactt
cctgtaccagcaacagggccgcctagacaagctgaccatcacctcccagaacctgcaactggagagccttcgcatgaagcttccg
aaatctgccaaacctgtgagccagatgcggatggctactcccttgctgatgcgtccaatgtccatggataacatgctccttgggcctgt
gaagaacgttaccaagtacggcaacatgacccaggaccatgtgatgcatctgctcacgaggtctggacccctggagtacccgcag
ctgaaggggaccttcccagagaatctgaagcatcttaagaactccatggatggcgtgaactggaagatcttcgagagctggatgaa
gcagtggctcttgtttgagatgagcaagaactccctggaggagaagaagcccaccgaggctccacctaaagagccactggacat
ggaagacctatcttctggcctgggagtgaccaggcaggaactgggtcaagtcaccctgtgcatgttggtgctgttgcctgatgaagtct
caggccttgagcagcttgagagtataatcaactttgaaaaactgactgaatggaccagttctaacgttatggaagagaggaagatc
aaagtgtacttacctcgcatgaagatggaggaaaaatacaacctcacatctgtcttaatggctatgggcattactgacgtgtttagctct
tcagccaatctgtctggcatctcctcagcagagagcctgaagatatctcaagctgtccatgcagcacatgcagaaatcaatgaagc
aggcagagaggtggtagggtcagcagaggctggagtggatgctgccacctgataagtcgacaatcaacctctggattacaaaattt
gtgaaagattgactggtattcttaactatgttgctccttttacgctatgtggatacgctgctttaatgcctttgtatcatgctattgcttcccgtat
ggctttcattttctcctccttgtataaatcctggttgctgtctctttatgaggagttgtggcccgttgtcaggcaacgtggcgtggtgtgcactg
tgtttgctgacgcaacccccactggttggggcattgccaccacctgtcagctcctttccgggactttcgctttccccctccctattgccacg
gcggaactcatcgccgcctgccttgcccgctgctggacaggggctcggctgttgggcactgacaattccgtggtgttgtcggggaag
ctgacgtcctttccatggctgctcgcctgtgttgccacctggattctgcgcgggacgtccttctgctacgtcccttcggccctcaatccagc
ggaccttccttcccgcggcctgctgccggctctagagcctcttccgcgtcttcgccttcccgggtcgagctcggtacctttaagaccaat
gacttacaaggcagctgtagatcttagccactifttaaaagaaaaggggggactggaagggctaattcactcccaacgaagacaa
gatctgctifttgcttgtactgggtctctctggttagaccagatctgagcctgggagctctctggctaactagggaacccactgcttaagc
ctcaataaagcttgccttgagtgcttcaagtagtgtgtgcccgtctgttgtgtgactctggtaactagagatccctcagacccttttagtca
gtgtggaaaatctctagcagtagtagttcatgtcatcttattattcagtatttataacttgcaaagaaatgaatatcagagagtgagagga
acttgtttattgcagcttataatggttacaaataaagcaatagcatcac
bd.huIDO.PGK.liAg
(SEQ ID NO: 158)
aaatttcacaaataaagcattiftttcactgcattctagttgtggtttgtccaaactcatcaatgtatcttatcatgtctggctctagctatcccg
cccctaactccgcccagttccgcccattctccgccccatggctgactaattifttttatttatgcagaggccgaggccgcctcggcctctg
agctattccagaagtagtgaggaggctffittggaggcctaggcttttgcgtcgagacgtacccaattcgccctatagtgagtcgtatta
cgcgcgctcactggccgtcgttttacaacgtcgtgactgggaaaaccctggcgttacccaacttaatcgccttgcagcacatccccctt
tcgccagctggcgtaatagcgaagaggcccgcaccgatcgcccttcccaacagttgcgcagcctgaatggcgaatggcgcgacg
cgccctgtagcggcgcattaagcgcggcgggtgtggtggttacgcgcagcgtgaccgctacacttgccagcgccctagcgcccgct
cctttcgctttcttcccttcctttctcgccacgttcgccggctttccccgtcaagctctaaatcgggggctccctttagggttccgatttagtgc
tttacggcacctcgaccccaaaaaacttgattagggtgatggttcacgtagtgggccatcgccctgatagacggthttcgccctttgac
gttggagtccacgttctttaatagtggactcttgttccaaactggaacaacactcaaccctatctcggtctattcttttgatttataagggattt
tgccgatttcggcctattggttaaaaaatgagctgatttaacaaaaatttaacgcgaattttaacaaaatattaacgtttacaatttcccag
gtggcacttttcggggaaatgtgcgcggaacccctatttgtttatttttctaaatacattcaaatatgtatccgctcatgagacaataaccct
gataaatgcttcaataatattgaaaaaggaagagtatgagtattcaacatttccgtgtcgcccttattccctiftttgcggcattttgccttcc
tgtttttgctcacccagaaacgctggtgaaagtaaaagatgctgaagatcagttgggtgcacgagtgggttacatcgaactggatctc
aacagcggtaagatccttgagagttttcgccccgaagaacgttttccaatgatgagcacttttaaagttctgctatgtggcgcggtattat
cccgtattgacgccgggcaagagcaactcggtcgccgcatacactattctcagaatgacttggttgagtactcaccagtcacagaaa
agcatcttacggatggcatgacagtaagagaattatgcagtgctgccataaccatgagtgataacactgcggccaacttacttctgac
aacgatcggaggaccgaaggagctaaccgctffittgcacaacatgggggatcatgtaactcgccttgatcgttgggaaccggagct
gaatgaagccataccaaacgacgagcgtgacaccacgatgcctgtagcaatggcaacaacgttgcgcaaactattaactggcga
actacttactctagcttcccggcaacaattaatagactggatggaggcggataaagttgcaggaccacttctgcgctcggcccttccg
gctggctggtttattgctgataaatctggagccggtgagcgtgggtctcgcggtatcattgcagcactggggccagatggtaagccctc
ccgtatcgtagttatctacacgacggggagtcaggcaactatggatgaacgaaatagacagatcgctgagataggtgcctcactga
ttaagcattggtaactgtcagaccaagtttactcatatatactttagattgatttaaaacttcatttttaatttaaaaggatctaggtgaagat
cctifttgataatctcatgaccaaaatcccttaacgtgagttttcgttccactgagcgtcagaccccgtagaaaagatcaaaggatcttct
tgagatcctifttttctgcgcgtaatctgctgcttgcaaacaaaaaaaccaccgctaccagcggtggtttgtttgccggatcaagagcta
ccaactctifttccgaaggtaactggcttcagcagagcgcagataccaaatactgtccttctagtgtagccgtagttaggccaccacttc
aagaactctgtagcaccgcctacatacctcgctctgctaatcctgttaccagtggctgctgccagtggcgataagtcgtgtcttaccgg
gttggactcaagacgatagttaccggataaggcgcagcggtcgggctgaacggggggttcgtgcacacagcccagcttggagcg
aacgacctacaccgaactgagatacctacagcgtgagctatgagaaagcgccacgcttcccgaagggagaaaggcggacagg
tatccggtaagcggcagggtcggaacaggagagcgcacgagggagcttccagggggaaacgcctggtatctttatagtcctgtcg
ggtttcgccacctctgacttgagcgtcgatttttgtgatgctcgtcaggggggcggagcctatggaaaaacgccagcaacgcggcctt
tttacggttcctggccttttgctggccttttgctcacatgttctttcctgcgttatcccctgattctgtggataaccgtattaccgcctttgagtga
gctgataccgctcgccgcagccgaacgaccgagcgcagcgagtcagtgagcgaggaagcggaagagcgcccaatacgcaaa
ccgcctctccccgcgcgttggccgattcattaatgcagctggcacgacaggtttcccgactggaaagcgggcagtgagcgcaacg
caattaatgtgagttagctcactcattaggcaccccaggctttacactttatgcttccggctcgtatgttgtgtggaattgtgagcggataa
caatttcacacaggaaacagctatgaccatgattacgccaagcgcgcaattaaccctcactaaagggaacaaaagctggagctg
caagcttggccattgcatacgttgtatccatatcataatatgtacatttatattggctcatgtccaacattaccgccatgttgacattgattatt
gactagttattaatagtaatcaattacggggtcattagttcatagcccatatatggagttccgcgttacataacttacggtaaatggcccg
cctggctgaccgcccaacgacccccgcccattgacgtcaataatgacgtatgttcccatagtaacgccaatagggactttccattgac
gtcaatgggtggagtatttacggtaaactgcccacttggcagtacatcaagtgtatcatatgccaagtacgccccctattgacgtcaat
gacggtaaatggcccgcctggcattatgcccagtacatgaccttatgggactttcctacttggcagtacatctacgtattagtcatcgct
attaccatggtgatgcggifttggcagtacatcaatgggcgtggatagcggtttgactcacggggatttccaagtctccaccccattgac
gtcaatgggagtttgttttggcaccaaaatcaacgggactttccaaaatgtcgtaacaactccgccccattgacgcaaatgggcggta
ggcgtgtacggtgggaggtctatataagcagagctcgtttagtgaaccggggtctctctggttagaccagatctgagcctgggagctc
tctggctaactagggaacccactgcttaagcctcaataaagcttgccttgagtgcttcaagtagtgtgtgcccgtctgttgtgtgactctg
gtaactagagatccctcagacccttttagtcagtgtggaaaatctctagcagtggcgcccgaacagggacctgaaagcgaaaggg
aaaccagagctctctcgacgcaggactcggcttgctgaagcgcgcacggcaagaggcgaggggcggcgactggtgagtacgc
caaaaattttgactagcggaggctagaaggagagagatgggtgcgagagcgtcagtattaagcgggggagaattagatcgcgat
gggaaaaaattcggttaaggccagggggaaagaaaaaatataaattaaaacatatagtatgggcaagcagggagctagaacg
attcgcagttaatcctggcctgttagaaacatcagaaggctgtagacaaatactgggacagctacaaccatcccttcagacaggatc
agaagaacttagatcattatataatacagtagcaaccctctattgtgtgcatcaaaggatagagataaaagacaccaaggaagcttt
agacaagatagaggaagagcaaaacaaaagtaagaccaccgcacagcaagcggccgctgatcttcagacctggaggagga
gatatgagggacaattggagaagtgaattatataaatataaagtagtaaaaattgaaccattaggagtagcacccaccaaggcaa
agagaagagtggtgcagagagaaaaaagagcagtgggaataggagctttgttccttgggttcttgggagcagcaggaagcacta
tgggcgcagcctcaatgacgctgacggtacaggccagacaattattgtctggtatagtgcagcagcagaacaatttgctgagggct
attgaggcgcaacagcatctgttgcaactcacagtctggggcatcaagcagctccaggcaagaatcctggctgtggaaagatacct
aaaggatcaacagctcctggggatttggggttgctctggaaaactcatttgcaccactgctgtgccttggaatgctagttggagtaata
aatctctggaacagattggaatcacacgacctggatggagtgggacagagaaattaacaattacacaagcttaatacactccttaat
tgaagaatcgcaaaaccagcaagaaaagaatgaacaagaattattggaattagataaatgggcaagtttgtggaattggtttaaca
taacaaattggctgtggtatataaaattattcataatgatagtaggaggcttggtaggtttaagaatagthttgctgtactttctatagtgaa
tagagttaggcagggatattcaccattatcgtttcagacccacctcccaaccccgaggggacccgacaggcccgaaggaatagaa
gaagaaggtggagagagagacagagacagatccattcgattagtgaacggatctcgacggtatcggttaacttttaaaagaaaag
gggggattggggggtacagtgcaggggaaagaatagtagacataatagcaacagacatacaaactaaagaattacaaaaaca
aattacaaaaattcaaaattttatcgatcacgagactagcctcgagagatctgatcataatcagccataccacatttgtagaggifttac
ttgctttaaaaaacctcccacacctccccctgaacctgaaacataaaatgaatgcaattgttgttgttaacttgtttattgcagcttataatg
gttacaaataaggcaatagcatcacaaatttcacaaataaggcattiftttcactgcattctagttttggthgtccaaactcatcaatgtat
cttatcatgtctggatctcaaatccctcggaagctgcgcctgtcatcgaattcctgcagcccggtgcatgactaagctagcagtctaag
gccaactcagaagagctttctcggttgtatcificacactccttaggaaagtcatgggattcgtacccccagtccctctgcttt
ccacatttgagggctcttccgacttgtcgccatcagtgggcttcttcttcgaaggificataatgtaagtatctactattgcgag
gtggaactttctcacagagaccagaccattcacacactcgttataagctttcgtcaagtcttcattgtgtcttgaaatgacaaa
ctcacggactgggggagctgactctaagaagaaaaggaagttccggtgggctggaggcatgtactctctcatttcctggaggaattc
tgcaggagattctttgccagcctcgtgttttattcccagaaggacatcaagactctggaagatgctgctctggcctgcactgccccctga
aaacatttttggggtgtcccagaccccctcatacagcagaccttctggcagcttggagctgcatttccagccagacagatatatgcgg
agaacgtggaaaaacgtgtctgggtccacaaagtcacgcatcctcttaaaaatttccttggctttctccagactggtagctatgtcgtgc
agtgccttttccaatgctttcaggtcttgacgctctactgcactggatacagtggggattgctttgattgcaggagaagctgcgatttccac
caatagagagacgaggaagaagcccttgtcgcagtccccaccaggaaatgagaacagaatgtccatgttctcgtatgtcatgggc
ccattggggtcctifttcttccagtttgccaggacacagtctgcataagacagaataggaggcaggcccaacttctctgagagctcgc
agtagggaacagcaatattgcggggcagcacctttcgaacatcgtcatcccctcggttccacacatacgccatggtgatgtacccca
gggccaggtgtgccaggcgctgtaacctgtgtcctctcagtccgtccgtgctcagtgtgggcagcttttcaacttcttctcgaagctgcc
cgttctcaatcagcacaggcagatttctagccacaaggacccaggggctgtatgcgtcgggcagctccaccagtggatgtggtaga
gcaaagcccacatcttcatctatgtggtggtcttcaaggattcttctagaaccttctgtaggagatattttactgagtgccatagtcggtcc
gctttgcggactgatggggctgcgctgcgctgcgctccagcgccccccctgcccgccggagctggccgcggcccgaattccgcgg
aggctggatcggtcccggtgtcttctatggaggtcaaaacagcgtggatggcgtctccaggcgatctgacggttcactaaacgagct
ctgcttatataggcctcccaccgtacacgcctaccctcgagaagcttgatatcgaattcccacggggttggggttgcgccttttccaag
gcagccctgggtttgcgcagggacgcggctgctctgggcgtggttccgggaaacgcagcggcgccgaccctgggtctcgcacatt
cttcacgtccgttcgcagcgtcacccggatcttcgccgctacccttgtgggccccccggcgacgcttcctgctccgcccctaagtcgg
gaaggttccttgcggttcgcggcgtgccggacgtgacaaacggaagccgcacgtctcactagtaccctcgcagacggacagcgc
cagggagcaatggcagcgcgccgaccgcgatgggctgtggccaatagcggctgctcagcggggcgcgccgagagcagcggc
cgggaaggggcggtgcgggaggcggggtgtggggcggtagtgtgggccctgttcctgcccgcgcggtgttccgcattctgcaagc
ctccggagcgcacgtcggcagtcggctccctcgttgaccgaatcaccgacctctctccccagggggatccaccatggatgaccaa
cgcgacctcatctctaaccatgaacagttgcccatactgggcaaccgccctagagagccagaaaggtgcagccgtggagctctgt
acaccggtgtctctgtcctggtggctctgctcttggctgggcaggccaccactgcttacttcctgtaccagcaacagggccgcctagac
aagctgaccatcacctcccagaacctgcaactggagagccttcgcatgaagcttccgaaatctgccaaacctgtgagccagatgc
ggatggctactcccttgctgatgcgtccaatgtccatggataacatgctccttgggcctgtgaagaacgttaccaagtacggcaacat
gacccaggaccatgtgatgcatctgctcacgaggtctggacccctggagtacccgcagctgaaggggaccttcccagagaatctg
aagcatcttaagaactccatggatggcgtgaactggaagatcttcgagagctggatgaagcagtggctcttgtttgagatgagcaag
aactccctggaggagaagaagcccaccgaggctccacctaaagagccactggacatggaagacctatcttctggcctgggagtg
accaggcaggaactgggtcaagtcaccctgtgtggcatctcctcagcagagagcctgaagatatctcaagctgtccatgcagcaca
tgcagaaatcaatgaagcaggcagagaggtggtagggtcagcagaggctggagtggatgctgcaagctgataagtcgacaatc
aacctctggattacaaaatttgtgaaagattgactggtattcttaactatgttgctccttttacgctatgtggatacgctgctttaatgcctttgt
atcatgctattgcttcccgtatggctttcattttctcctccttgtataaatcctggttgctgtctctttatgaggagttgtggcccgttgtcaggca
acgtggcgtggtgtgcactgtgtttgctgacgcaacccccactggttggggcattgccaccacctgtcagctcctttccgggactttcgc
tttccccctccctattgccacggcggaactcatcgccgcctgccttgcccgctgctggacaggggctcggctgttgggcactgacaatt
ccgtggtgttgtcggggaagctgacgtcctttccatggctgctcgcctgtgttgccacctggattctgcgcgggacgtccttctgctacgt
cccttcggccctcaatccagcggaccttccttcccgcggcctgctgccggctctagagcctcttccgcgtcttcgccttcccgggtcgag
ctcggtacctttaagaccaatgacttacaaggcagctgtagatcttagccactifttaaaagaaaaggggggactggaagggctaatt
cactcccaacgaagacaagatctgctifttgcttgtactgggtctctctggttagaccagatctgagcctgggagctctctggctaacta
gggaacccactgcttaagcctcaataaagcttgccttgagtgcttcaagtagtgtgtgcccgtctgttgtgtgactctggtaactagagat
ccctcagacccttttagtcagtgtggaaaatctctagcagtagtagttcatgtcatcttattattcagtatttataacttgcaaagaaatgaa
tatcagagagtgagaggaacttgtttattgcagcttataatggttacaaataaagcaatagcatcac
hulL-10 (DNA)
(SEQ ID NO: 159)
atgcacagctcagcactgctctgttgcctggtcctcctgactggggtgagggccagcccaggccagggcacccagtctgagaaca
gctgcacccacttcccaggcaacctgcctaacatgcttcgagatctccgagatgccttcagcagagtgaagactttctttcaaatgaa
ggatcagctggacaacttgttgttaaaggagtccttgctggaggactttaagggttacctgggttgccaagccttgtctgagatgatcca
gifttacctggaggaggtgatgccccaagctgagaaccaagacccagacatcaaggcgcatgtgaactccctgggggagaacct
gaagaccctcaggctgaggctacggcgctgtcatcgatttcttccctgtgaaaacaagagcaaggccgtggagcaggtgaagaat
gcctttaataagctccaagagaaaggcatctacaaagccatgagtgagtttgacatcttcatcaactacatagaagcctacatgaca
atgaagatacgaaactga
hulL-10 (protein)
(SEQ ID NO: 160)
MHSSALLCCLVLLTGVRASPGQGTQSENSCTHFPGNLPNMLRDLRDAFSRVKTFFQMKDQL
DNLLLKESLLEDFKGYLGCQALSEMIQFYLEEVMPQAENQDPDIKAHVNSLGENLKTLRLRLR
RCHRFLPCENKSKAVEQVKNAFNKLQEKGIYKAMSEFDIFINYIEAYMTMKIRN*
hulDO (DNA)
(SEQ ID NO: 161)
atggcccatgccatggaaaacagctggaccatcagcaaagagtaccacatcgacgaggaagtgggcttcgccctgcctaatcctc
aagagaacctgcctgacttctacaacgactggatgtttatcgccaaacatctgcccgacctgatcgagagcggccagctgagaga
aagagtggaaaagctgaacatgctgagcatcgaccacctgaccgaccacaagtctcagagactggccagactggtgctgggctg
tatcaccatggcctacgtgtggggaaaaggccatggcgacgtgcggaaagtgctgcccagaaatatcgccgtgccttactgccag
ctgtccaagaagctggaactgcctcctatcctggtgtacgccgattgcgtgctggccaactggaagaagaaggaccccaacaagc
ccctgacctacgagaacatggacgtgctgtttagcttccgcgacggcgattgcagcaagggattcttcctggtgtccctgctggtgga
aatcgccgctgcctctgccatcaaagtgatccccaccgtgttcaaggccatgcagatgcaagagcgggacaccctgctgaaggcc
ctgctggaaattgcctcctgcctggaaaaagccctccaggtgttccaccagatccacgaccacgtgaaccccaaggccttcttcagc
gtgctgcggatctatctgtctggctggaagggcaatccccagctgtctgacggcctggtgtatgaaggcttctgggaagatcccaaag
agttcgctggcggctctgccggacagtctagtgtgttccagtgcttcgatgtgctgctgggcatccagcaaacagccggcggaggac
atgctgctcagtttctgcaagacatgcggcggtacatgcctccagctcaccggaactttctgtgcagcctggaaagcaaccccagcg
tgcgggaattcgtgctgtctaaaggcgacgccggactgagagaagcctacgatgcctgtgtgaaggctctggtgtctctgcggagct
accacctccagatcgtgaccaagtacattctgatccccgccagccagcagcctaaagagaacaagaccagcgaggacccctcc
aagctggaagcaaaaggcacaggcggaaccgatctgatgaacttcctgaaaaccgtgcggtccaccaccgagaagtctctgctg
aaagagggctga
hulDO (protein)
(SEQ ID NO: 162)
MAHAMENSWTISKEYHIDEEVGFALPNPQENLPDFYNDWMFIAKHLPDLIESGQLRERVEKLN
MLSIDHLTDHKSQRLARLVLGCITMAYVWGKGHGDVRKVLPRNIAVPYCQLSKKLELPPILVYA
DCVLANWKKKDPNKPLTYENMDVLFSFRDGDCSKGFFLVSLLVEIAAASAIKVIPTVFKAMQM
QERDTLLKALLEIASCLEKALQVFHQIHDHVNPKAFFSVLRIYLSGWKGNPQLSDGLVYEGFW
EDPKEFAGGSAGQSSVFQCFDVLLGIQQTAGGGHAAQFLQDMRRYMPPAHRNFLCSLESNP
SVREFVLSKGDAGLREAYDACVKALVSLRSYHLQIVTKYILIPASQQPKENKTSEDPSKLEAKG
TGGTDLMNFLKTVRSTTEKSLLKEG*
gagpol polyprotein Simian immunodeficiency virus (Vpx) (DNA)
(SEQ ID NO: 163)
atgggcgcgagaaactccgtcttgtcagggaagaaagcagatgaattagaaaaaattaggctacgacccggcggaaagaaaa
agtacatgttgaagcatgtagtatgggcagcaaatgaattagatagatttggattagcagaaagcctgttggagaacaaagaagga
tgtcaaaaaatactttcggtcttagctccattagtgccaacaggctcagaaaatttaaaaagcctttataatactgtctgcgtcatctggt
gcattcacgcagaagagaaagtgaaacacactgaggaagcaaaacagatagtgcagagacacctagtggtggaaacaggaa
cagcagaaactatgccaaaaacaagtagaccaacagcaccatctagcggcagaggaggaaattacccagtacaacaaatagg
tggtaactatgtccacctgccattaagcccgagaacattaaatgcctgggtaaaattgatagaggaaaagaaatttggagcagaag
tagtgccaggatttcaggcactgtcagaaggctgcaccccctatgacattaatcagatgttaaattgtgtgggagaccatcaagcggc
tatgcagattatcagagatattataaatgaggaggctgcagattgggacttgcagcacccacaaccagctccacaacaaggacag
cttagggagccgtcaggatcagatattgcaggaacaactagttcagtagatgaacaaatccagtggatgtacagacaacagaacc
ccataccagtaggcaacatttacaggagatggatccaactggggttgcaaaaatgtgtcagaatgtataacccaacaaacattcta
gatgtaaaacaagggccaaaagagccatttcagagctatgtagacaggttctacaaaagcttaagagcagaacaaacagatgc
agcagtaaagaattggatgactcaaacactgctgattcaaaatgctaacccagattgcaagctagtgctgaaggggctgggtgtga
atcccaccctagaagaaatgctgacggcttgtcaaggagtagggggaccaggacagaaggctagattaatggcagaagccctg
aaagaggccctcgcaccagtgccaatcccttttgcagcagcccagaagaggggaccaagaaagccaattaagtgttggaattgtg
ggaaggagggacactctgcaaggcaatgcagagccccaagaagacagggatgctggaaatgtggaaaaatggaccatgttat
ggccaaatgcccagacagacaggcgggttttttaggccttggtccatggggaaagaagccccgcaatttccccatggctcaagtgc
atcaggggctgacgccaactgctcccccagaggacccagctgtggatctgctaaagaactacatgcagttgggcaagcagcaga
gagaaagcagagagaagccttacaaggaggtgacagaggatttgctgcacctcaattctctctttggaggagaccagtagtcactg
ctcatattgaaggacagcctgtagaagtattattggatacaggggctgatgattctattgtaacaggaatagagttaggtccacattata
ccccaaaaatagtaggaggaataggaggifttattaatactaaagaatacaaaaatgtaaaaatagaagttttaggcaaaaggatt
aaagggacaatcatgacaggggacactccgattaacatttttggtaggaatttgctaacagctctggggatgtctctaaatcttcccat
agctaaggtagagcctgtaaaagtcaccttaaagccaggaaaggttggaccaaaattgaagcagtggccattatcaaaagaaaa
gatagttgcattaagagaaatctgtgaaaagatggaaaaggatggtcagttggaggaagctcccccgaccaatccatacaacacc
cccacatttgccataaagaaaaaagataagaacaaatggagaatgctgatagattttagggaactaaatagggtcactcaggactt
tacagaagtccaattaggaataccacaccctgcaggactagcaaaaaggaaaaggattacagtactggatataggtgatgcatatt
tctccatacctctagatgaagaatttaggcagtacactgcctttactttaccatcagtaaataatgcagagccaggaaaacgatacattt
ataaggttctgcctcagggatggaaggggtcaccagccatcttccaatacactatgagacatgtgctagaacccttcaggaaggca
aatccagatgtgaccttagtccagtatatggatgacatcttaatagctagtgacaggacagacctggaacatgacagggtagttttac
agctaaaggaactcttaaatagcatagggttctctaccccagaagagaaattccaaaaagatcccccatttcaatggatggggtac
gaattgtggccgacaaaatggaagttgcaaaagatagagttgccacaaagagagacctggacagtgaatgatatacagaagtta
gtaggagtattaaattgggcagctcaaatttatccaggtataaaaaccaaacatctctgtaggttaattagaggaaaaatgactctaa
cagaggaagttcagtggactgagatggcagaagcagaatatgaggaaaataagataattctcagtcaggaacaagaaggatgtt
attaccaagaaggcaagccattagaagccacggtaataaagagtcaggacaatcagtggtcttataaaattcaccaagaagaca
aaatactgaaagtaggaaaatttgcaaagataaagaatacacataccaatggagttagactattagcacatgtaatacagaaaat
aggaaaggaagcaatagtgatctggggacaggtcccaaaattccacttaccagttgagagggatgtatgggaacagtggtggac
agactattggcaggtaacctggataccggagtgggattttatctcaacgccaccactagtaagattagtcttcaatctagtgaaggac
cctatagagggagaagaaacctattatacagatggatcatgtaataaacagtcaaaagaagggaaagcaggatatatcacagat
aggggcaaagacaaagtaaaagtgttagaacagactactaatcaacaagcagaattagaagcatttctcatggcattgacagact
cagggccaaagacaaatattatagtagattcacaatatgttatgggaataataacaggatgccctacagaatcagagagcaggct
agttaaccaaataatagaagaaatgattaaaaagtcagaaatttatgtagcatgggtaccagcacacaaaggtataggaggaaa
ccaagaaatagaccacctagttagtcaggggattagacaagttctcttcttggaaaagatagagccagcacaagaagaacatgat
aaataccatagtaatgtaaaagaattggtattcaaatttggattacccagaatagtggccagacagatagtagacacctgtgataaat
gtcatcagaaaggagaagctatacatgggcaggtaaattcagatctagggacttggcaaatggactgtacccatctagaaggaaa
aatagtcatagttgcagtacatgtagctagtggattcatagaagcagaagtaattccacaagagacaggaagacagacagcacta
tttctgttaaaattggcaggcagatggcctattacacatctacacacagataatggtgctaactttgcctcgcaagaagtaaagatggtt
gcatggtgggcagggatagagcacacctttggggtaccatacaatccacagagtcagggagtagtggaagcaatgaatcaccac
ctgaaaaatcaaatagatagaatcagggaacaagcaaattcagtagaaaccatagtattaatggcagttcattgcatgaattttaaa
agaaggggaggaataggggatatgactccagcagaaagattaattaacatgatcactacagaacaagaaatacaatttcaacaa
tcaaaaaactcaaaatttaaaaattttcgggtctattacagagaaggcagagatcaactgtggaagggacccggtgagctattgtgg
aaaggggaaggagcagtcatcttaaaggtagggacagacattaaggtagtacccagaagaaaggctaaaattatcaaagattat
ggaggaggaaaagaggtggatagcagttcccacatggaggataccggagaggctagagaggtggcatag
gagpol polyprotein Simian immunodeficiency virus (Vpx) (Protein)
(SEQ ID NO: 164)
MGARNSVLSGKKADELEKIRLRPGGKKKYMLKHVVWAANELDRFGLAESLLENKEGCQKILS
VLAPLVPTGSENLKSLYNTVCVIWCIHAEEKVKHTEEAKQIVQRHLVVETGTAETMPKTSRPTA
PSSGRGGNYPVQQIGGNYVHLPLSPRTLNAVVVKLIEEKKFGAEVVPGFQALSEGCTPYDINQ
MLNCVGDHQAAMQIIRDIINEEAADWDLQHPQPAPQQGQLREPSGSDIAGTTSSVDEQIQWM
YRQQNPIPVGNIYRRWIQLGLQKCVRMYNPTNILDVKQGPKEPFQSYVDRFYKSLRAEQTDA
AVKNWMTQTLLIQNANPDCKLVLKGLGVNPTLEEMLTACQGVGGPGQKARLMAEALKEALAP
VPIPFAAAQKRGPRKPIKCWNCGKEGHSARQCRAPRRQGCWKCGKMDHVMAKCPDRQAG
FLGLGPWGKKPRNFPMAQVHQGLTPTAPPEDPAVDLLKNYMQLGKQQRESREKPYKEVTED
LLHLNSLFGGDQ

### Examples

Alternative strategies have been developed to generate tolerogenic DC (toILV-DC) based on LV-mediated gene transfer of specific Ag-derived peptide(s) coupled with target sequences for miR155 and miR146a, known regulators of DC maturation (DC-Ag.miRNA), with IL-10 (DC-IL-10/Ag), or IDO (DC-IDO/Ag) (FIG. 1). To define the mode of action of toILV-DC, the inventors used Ovalbumin (OVA) as model Ag. LVs encoding for Ii fused with OVA₃₁₅₋₃₅₃, which contain OVA₃₂₃₋₃₃₉ recognized by TCR transgenic OTII CD4⁺ T cells, were generated and used to transduce bone marrow (BM) cells during DC differentiation. LV.IiOVA₃₁₅₋₃₅₃.miR155T.miR146aT, LV.IL-10.IiOVA₃₁₅₋₃₅₃, LV.IDO.IiOVA₃₁₅₋₃₅₃, and as control LV.IiOVA₃₁₅₋₃₅₃ were used to obtain the following LV-DC: DC-OVA, DC-OVA.miRNA, DC-IL-10/OVA, DC-IDO/OVA. LV-DC were CD11c⁺ and expressed CD80, CD86, and MHC class II at the same levels of un-transduced DC (FIG. 2). DC-OVA promoted proliferation of OTII CD4⁺ T cells, while DC-IL-10/OVA induced a low OTII CD4⁺ T cell proliferative response. Conversely, proliferation induced by DC-IDO/OVA was comparable to that induced by DC-OVA (FIG. 3). Notably, T cells generated with DC-IL-10/OVA were anergic in response to secondary OVA stimulation (FIG. 4), suggesting that transduction of DC with LV.IL-10.IiOVA promotes a population of DC that are functionally super-imposable to DC-10, a population of cells generated in vitro in the presence of IL-10 that efficiently promote anergic Ag-specific T cells ((22); WO2007/131575; US2016/0046910 A1). DC-OVA.miRNA promoted OTII CD4⁺ T cell proliferation, but, upon LPS activation the post-transcriptional regulation mediated by miR155 and miR146a abrogated their ability to promote OTII CD4⁺ T cell proliferation (FIG. 5), indicating that DC-OVAmiRNA present OVA to responding CD4⁺ T cells only at immature-like stage.

To study the mechanism of action of LV-DC, the inventors developed chimeric mice by transplanting CD45.1 (95%) and CD45.2 OTII/FirTiger (5%) bone marrow (BM) cells into lethally irradiated CD45.1 mice. OTII/FirTiger CD4⁺ T cells are TCR transgenic cells recognizing OVA₃₂₃₋₃₃₉ and expressing RFP and GFP as reporter genes for foxp3 and Il10, respectively. At full reconstitution, chimeric mice with ˜5% of OTII/FirTiger CD4⁺ T cells in circulation (FIG. 6, left panel) were repetitively injected with the different subsets of LV-DC. Five weeks after the last DC injection, the frequency OVA-specific CD45.2 OTII CD4⁺ T cells was significantly higher in the spleen of mice treated with DC-OVA compared to those injected with DC-GFP (FIG. 6, right panel). Moreover, the expansion of OVA-specific CD4⁺ T cells was evident in mice receiving the different tolerogenic LV-DC encoding for OVA, but not GFP. In addition, in mice treated with DC-OVA or tolerogenic LV-DC expressing OVA and tolerogenic molecules the inventors observed the expansion of CD4⁺ memory T cells (not shown), indicating that in vivo priming of OVA-specific T cells occurs. The inventors next investigated the induction of OVA-specific Tregs in treated chimeric mice using the reporter genes and expression of Tr1 specific markers, CD49b and LAG-3 (90), and of CD25 for Foxp3 Tregs. Results showed that injection of DC-OVA.miRNA or DC-IL-10/OVA promoted a significantly higher expansion of IL-10-producing CD49b⁺ LAG-3+Tr1 cells as compared to that observed in mice treated with DC-GFP or DC-OVA (FIG. 7). Conversely, none of the LV-DC treatments induced a significant expansion of FOXP3⁺ Tregs (not shown). Upon in vitro re-stimulation with DC-OVA, T cells isolated from the spleen of tolerogenic LV-DC-treated mice were hypo-responsive, as demonstrated by the low proliferative capacity of OTII CD4⁺ T cells as compared to that observed by T cells isolated from mice injected with DC-OVA (FIG. 8).

With the aim at modulating both CD4+ and CD8+ T cell responses, the inventors generated LV encoding for OVA₂₄₂₋₃₅₃, which contains OVA₃₂₃₋₃₃₉ recognized by TCR transgenic OTII CD4⁺ T cells and OVA₂₅₇₋₂₆₄ (SIINFEKL) by TCR transgenic OTI CD8⁺ T cells. BM cells were transduced with either LV.IiOVA₃₁₅₋₃₅₃ or LV.IiOVA₂₄₂₋₃₅₃, and engineered DC-OVA₃₁₅₋₃₅₃ and DC-OVA₂₄₂₋₃₅₃ were used to stimulate OTII and OTI cells. Both DC-OVA promoted the proliferation of OTII CD4+ T cells, whereas DC-OVA₂₄₂₋₃₅₃, but not DC-OVA₃₁₅₋₃₅₃, promoted the proliferation of OTI cells (FIG. 9). These data indicate that LV-DC can be engineered to modulate both CD4⁺ and CD8⁺ T cell responses.

These results show that LV-mediated gene transfer of Ag fused to invariant chain endorses DC with the ability to present and promote Ag-specific CD4⁺ and CD8⁺ T cell proliferation in vitro and in vivo. Moreover, addition of tolerogenic elements (miRNA target sequences, IL-10 or IDO) in the LV backbone, ensuring encoded Ag presentation by immature-like DC or by DC in the presence of high levels of IL-10 and IDO, favors the generation of regulatory DC that promote Ag-specific T cell hypo-responsiveness, and, in the case of DC-OVA.miRNA or DC-IL-10/OVA expansion of Ag-specific Tr1 cells.

To study efficacy of LV-DC to modulate diabetogenic T cell responses in vitro and in vivo, LV encoding for Ii fused with InsB₄₋₂₉, containing the diabetogenic peptide InsB₉₋₂₃ alone or in combination with miRNA155 and 146a target sequences, IL-10, or IDO were generated and used to transduce BM cells isolate from NOD mice during DC differentiation. LV.IiInsB₄₋₂₉.miR155T.miR146aT, LV.IL-10.IiInsB₄₋₂₉, LV.IDO.IiInsB₄₋₂₉, and as control LV.IiInsB₉₋₂₃ and LV.IiOVA₃₁₅₋₃₅₃ were used to obtain DC-InsB.miRNA, DC-IL-10/InsB, DC-IDO/InsB, DC-InsB and DC-OVA. LV-DCs were CD11c⁺ and expressed the MHC class II I-Ag^{g7} and CD86 at similar levels to those expressed by un-transduced DCs (not shown). CD4⁺ T cells isolated from a diabetic NOD mouse proliferated when stimulated with DC-InsB, but not with DC-OVA (FIG. 10). Similar to results obtained with OVA, DC-IL-10/InsB promoted a lower CD4⁺ T cell proliferation as compared to control DC-InsB. Conversely, T cells stimulated with DC-InsBmiRNA and DC-IDO/InsB proliferated as much as cells stimulated with DC-InsB (FIG. 10).

The inventors next investigated the biodistribution and survival of LV-DC in vivo. Thus, BM cells isolated from Balb/c mice were transduced with LV-encoding for luciferase on day 2 during DC differentiation. LV-DC^{luc} were intraveneously (i.v.) or intraperitoneally (i.p.) injected in Balb/c recipient mice and biodistribution and LV-DC^{luc} survival was monitored by bioluminescence imaging (BLI). As expected, upon i.v. or i.p. injection LV-DC^{luc} localized in lung and peritoneum, respectively. I.v. injected LV-DC^{luc} localized in the spleen Starting from day 6, whereas i.p. injected LV-DC^{luc} localized in the spleen starting from day 2. Injected cells progressively disappeared by day 8-10 (FIG. 11). Study the efficacy of LV-DC in preventing T1D development by injecting cells i.p. To this end, 10 week-old NOD female mice were repetitively injected with DC-InsB, DC-InsBmiRNA, DC-IL-10/InsB, DC-IDO/InsB, and DC-OVA. Results showed that IDO constitutive expression by DC-IDO/InsB significantly reduced T1D development in NOD mice as compared to control mice treated with DC-OVA (p=0.0028) or DC-InsB (p=0.0407) (FIG. 11). Administration of DC-IL-10/InsB resulted in a milder, but not significant control of the disease, while DC-InsBmiRNA-treated NOD mice showed delayed T1D onset as compared to DC-OVA-treated controls.

Treated mice were sacrificed 15 weeks post the last DC injection and the frequency of Treg in the spleen and pancreatic lymph nodes was analyzed. Overall, no specific induction of CD49b⁺ LAG-3⁺ Tr1 cells or CD25⁺Foxp3⁺ Tregs (FIG. 12), and high variability in the proliferative response to InsB₉₋₂₃ by CD4⁺ T cells isolated from LV-DC-treated mice, independently from the subtype of LV-DC injected, were observed (not shown).

In conclusion, the inventors developed an efficient and powerful method to generate stable and effective tolerogenic DC by cutting-edge technology based on LV encoding for specific autoAg and tolerogenic molecules.

To translate the LV based approach to human cells the inventors first developed an efficient protocol for promoting bdLV-mediated transduction of human DC. To this end, CD14⁺ cells were pre-treated or not with viral-like particles containing the simian immunodeficiency virus (SIV)-derived accessory protein Vpx-VPL to counteract SAMHD1-mediated restriction on day 0, 2, or 5 during DC differentiation (FIG. 13 left panel). Pre-treatment with Vpx-VPL at all time points analyzed improved transduction efficiency reaching the higher efficiency when cells were pre-treated with Vpx-VPL at day 0 (FIG. 13 right panel). Importantly, Vpx-VPL pre-treatment performed on day 0 did not affect the activation of resulting cells at the end of the culture (FIG. 14). Time course analysis demonstrated that 1 h exposure to Vpx-VPL is sufficient to reach 95% of transduction efficiency (FIG. 38).

Using the established protocol to generate engineered human LV-DC, the inventors first investigated the ability of LV co-encoding for IL-10 and ΔNGFR a marker for selection previously used to generate Tr1-like (CD4^{IL-10}) cells ((40, 85) WO2016146542) to generate DC^{IL-10}. CD14⁺ cells were treated with Vpx-VPL for 6-8 hours and then transduced with LV-IL-10/ΔNGFR (DC^{IL-10}) or LV-GFP/ΔNGFR (DC^{GFP}) at day 0 during DC differentiation. As control the inventors used DC untransduced (DC^{UT}) and DC-10 differentiated from the same donors by culturing CD14⁺ cells with GM-CSF and IL-4 or GM-CSF, IL-4, and IL-10, respectively. Human DC were efficiently transduced by both vectors, reaching up to 98% of transduction, as demonstrated ΔNGFR expression (FIG. 15 left panel). Analysis of the expression of DC-10-associated markers demonstrated that DC^{IL-10} expressed CD14, CD16, CD141, CD163, ILT4 and HLA-G, while control DC^{UT} of DC^{GFP} cells did not (FIG. 15 right panel). DC^{IL-10} secreted significantly higher levels of IL-10 compared to DC-10 at steady state and upon activation. Importantly, DC^{IL-10}, similar to DC-10, do not produce IL-12 upon activation (FIG. 16). DC^{IL-10}, similar to DC-10, promoted hypo-responsiveness in allogeneic T cells, both CD4⁺ and CD8⁺ T cells (FIG. 17). The inventors next compared the ability of DC^{IL-10} to promote anergic allo-specific Tr1 cells to that of DC-10. To this end, allogeneic CD4⁺ T cells were stimulated for 10 days with DC^{IL-10}, or, as control, DC^{GFP}, DC^{UT} and DC-10. In all donors tested, CD4⁺ T cells primed with DC^{IL-10} (T-DC^{IL-10}), similar to cells activated with DC-10 (T-DC-10), re-stimulated with the same alloAg used for their priming were anergic compared to T cell primed with DC^{UT} (T-DC^{UT}) or DC^{GFP} (T-DC^{GFP}) (FIG. 18). Moreover, T-DC^{IL-10} and T-DC-10 cells contained a significantly higher proportion of Tr1 cells compared to T-DC^{UT} and T-DC^{GFP} cells (FIG. 19, left panel). T-DC^{IL-10} cells when re-stimulated with the same alloAg used for their priming secreted significantly higher levels of IL-10 compared to T-DC-10, T-DC^{UT} and T-DC^{GFP} cells (FIG. 19, right panel).

Overall these findings indicate that LV-mediated IL-10 gene transfer convert human DC in DC-10-like cells endowed with the ability to modulate allogeneic T cells and promote the differentiation of anergic allo-specific Tr1 cells.

To study the ability of DC^{IL-10} to prevent graft-versus host disease (GvHD) the inventors generated murine DC^{IL-10} by transducing BM cells isolated from Balb/c mice with LV-IL-10/ΔNGFR during DC differentiation. As control, BM cells transduced with LV-GFP/ΔNGFR (DC^{GFP}) were generated. Murine DC^{IL-10} and DC^{GFP} were then adoptively transferred into Balb/c mice lethelly irradiated and injected with allogeneic (C57Bl/6) BM cells and splenocytes. Untreated mice or mice treated with DC^{GFP} developed lethal GvHD, whereas single injection of DC^{IL-10} significantly delayed GvHD (FIG. 20).

To generate Ag-specific human LV-DC, the inventors designed LV constructs encoding for human CLIP sequence of Iip33 (invariant chain p33 binding domain for MHC class II molecules) fused with autoAg-derived peptides. The inventors generated LV encoding for Iip33 fused with Insulin B4-29 sequence (LV.InsB₄₋₂₉), or with α2-gliadin 51-80 (LV.Glia₅₁₋₈₀). DC differentiating monocytes were transduced with LV using an optimized protocol which foresees the pre-treatment of CD14⁺ precursors with Vpx-VPL in serum free medium (FIG. 21). After differentiation, differentiation of DC was monitored by the expression of DC-SIGN. As depicted in FIG. 22, human LV-transduced cells are DC-SIGN⁺, and in case of LV-IL-10/Ag-transduced cells (DC-IL-10/Ag) resulting DC also expressed CD14. Transduction efficiency was assessed with ΔNGFR expression in case of control LV-ΔNGFR/Ag-transduced cells. In case of LV-IL-10/Ag-transduced cells transduction efficiency was monitored by intracytoplasmic staining for IL-10. Specifically, DC-IL-10/Ag and, as control, DC transduced with LV-CLIP (DC^{CLIP}) or with LV-ΔNGFR/Ag (DC-Ag) were left unstimulated or activated with LPS/IFN-g for 24 hrs and stained for IL-10. Results showed that DC-IL-10/Ag expressed IL-10, at steady state and upon activation, whereas, only 4-10% of DC^{CLIP} and DC-Ag expressed IL-10 only after stimulation (FIG. 23). In case of LV-IDO/Ag-transduced cells, transduction efficiency was monitored by IDO expression. As depicted in FIG. 24, DC-IDO/Ag expressed IDO whereas DC^{CLIP} and DC-Ag barely expressed IDO.

Similar to DC^{IL-10}, DC-IL-10/Ag express high levels of DC-10-associated markers including CD14, CD141, CD163, and ILT4, whereas do not acquire the expression of HLA-G (FIG. 25). Conversely, DC-IDO/Ag are phenotypically similar to DC^{UT}, DC^{CLIP} and DC-Ag (not shown). Cytokine production profile of DC-IL-10/Ag demonstrated that, similar to DC^{IL-10} and DC-10, these cells secreted significantly higher levels of IL-10 at steady state and upon LPS/IFN-g stimulation, and low levels of IL-12 (FIG. 26 and FIG. 27). DC-IDO/Ag displayed a cytokine profile super-imposable to that of to DC^{UT}, DC^{CLIP} and DC-Ag (not shown).

Functional characterization of DC-IL-10/Ag demonstrated that in contrast to DC-Ag that consistently induced Ag-specific proliferative responses in HLA-restricted T cells, concomitant over-expression of IL-10 down-regulated the proliferation of Ag-specific T cells (FIG. 28). Importantly, stimulation of autologous T cells with DC-IL-10/Ag for 10 days, promoted the induction of Ag-specific anergic T cells that contained high frequency of CD49b+LAG-3+Tr1 cells (FIG. 29). DC-IDO/Ag promoted proliferation of autologous T cells similar to that induced by control DC-Ag (FIG. 30), and stimulation of autologous T cells with DC-IDO/Ag for 10 days, promoted the induction of a population of cells containing high proportion of FOXP3⁺CTLA4⁺ cells (FIG. 31).

Overall these data demonstrated that engineered DC with LVs encoding for invariant chain (Ii) fused to a specific Ag coupled with multiple target sequences for miR155 and miR146a, known regulators of DC maturation (DC-Ag.miRNA) or with IL-10 (DC-IL-10/Ag); or IDO (DC-IDO/Ag) generated a population of tolerogenic DC able to modulate Ag-specific T cell responses and to promote the differentiation of Ag-specific Tr1 cells or FOXP3⁺ T cells in vitro and in vivo. According to the above evidence, a strong inhibition of T effector cells and/or a strong activation of T regulatory cells may be obtained using the exemplified approaches.

Being DC^{IL-10} similar to DC-10, we investigated their ability to promote allo-specific Tr1 cells in vitro by stimulating allogeneic CD4⁺ T cells for 10 days. In all donors tested, CD4⁺ T cells primed with DC^{IL-10}, [T(DC^{IL-10}) cells], contained a higher proportion of Tr1 cells compared to T cells primed with DC^{UT} and DC^{GFP} [T(DC^{UT}) and T(DC^{GFP}) cells, respectively] (FIG. 19). T(DC^{IL-10}) cells re-stimulated with mature DC (mDC) autologous to DC used for priming proliferated at lower levels compared to T(DC^{UT}) and T(DC^{GFP}) cells (FIG. 32), produced significantly higher level of IL-10 compared to both (T-DC^{UT}) and (T-DC^{GFP}) cells, but similar levels of IFN-γ. Finally, T(DC^{IL-10}) cells suppressed the proliferation of autologous CD4⁺ T cells with mDC from the same donor of DC used for priming, with a suppression of 67% on average (FIG. 33). Overall these findings indicate that LV-mediated IL-10 gene transfer in human DC promotes the generation of human DC^{IL-10} endowed with the ability to modulate allogeneic T cell responses and promote the differentiation of allo-specific Tr1 cells in vitro.

To assess the modulatory activity of DC^{IL-10} in vivo the inventors took advantage of the recently developed protocol for the repopulation of NSG mice with human cord blood CD34⁺ cells. Intra-liver injection of human CD34⁺ cells in sub-lethally irradiated neonate NSG mice allowed efficient engraftment of human CD45⁺ T cells in bone marrow (BM) and differentiation of lymphoid (B, T effector and T regulatory) and myeloid mature cells in the periphery (91). Reconstituted huNSG mice were immunized by i.v. injection of irradiated allogeneic human APC and boosted 7 days after with autologous DC^{UT} alone or with DC^{IL-10} (DC^{UT}+DC^{IL10}) or DC^{GFP} (DC^{UT}+DC^{GFP}) (FIG. 34). Treatment with DC^{IL-10} prevented the in vivo proliferation of CD4⁺ T cells, assessed by Ki67 staining, induced by allogeneic DC^{UT} (FIG. 34). These data demonstrated that human DC^{IL-10} modulate allogeneic T cell responses in vivo.

One of the key aspects of DC-based cell products is their stability (i.e. the expression of specific markers, secretion of cytokines, stimulatory activity and induction of Tr1 cells are maintained after activation), the inventors therefore assessed the phenotype of DC^{IL-10} after in vitro stimulation with different TLR agonists (i.e. LPS, Listeria, Flagellin, Poli I:C, and CpG) or with a mixed of pro-inflammatory cytokines (IL-1b, TNF-a and IL-6). Similar to previous data obtained in DC-10 (92), CD163 and CD141 were firmly expressed on DC^{IL-10} upon activation (FIG. 35). Conversely, the CD16 expression is affected by activation with LPS or listeria (FIG. 35). No major changes in the expression of CD14 and CD1a were observed in activated DC^{IL-10} compared to not stimulated DC^{IL-10} (FIG. 35). The expression of CD86 is significantly up-regulated DC^{IL-10} upon LPS, Listeria, and CpG stimulation, while not effect on CD83, and HLA-DR expression was observed (FIG. 35). While HLA-G expression in DC^{IL-10} remained stable upon activation (not shown), the expression of ILT4 significantly increased and decreased upon LPS and Listeria or Poli I:C and CpG stimulation, respectively (FIG. 36). Being ILT4 critically involved in DC-10-mediated induction of Tr1 cells (22), the inventors selected LPS and Poli I:C to stimulate DC^{IL-10} and investigate their tolerogenic activity in vitro. Independently from the stimuli, activated DC^{IL-10} secreted at steady state and upon LPS/IFNg stimulation huge amounts of IL-10 in the absence of IL-12. The percentage of induced Tr1 cells in DC^{IL-10} culture was lower upon TLR stimulation, but it was still higher compared to the DC^{GFP} culture (FIG. 36). T(stim-DC^{IL-10}) were anergic when re-stimulated with mDC autologous to DC used for priming, even if their anergy was less pronounced compared to T(unstim-DC^{IL-10}). Similarly, the levels of IL-10 production were lower in T(stim-DC^{IL-10}) compared to T(unstim-DC^{IL-10}), but higher compared to T(DC^{GFP}), (FIG. 37). Regardless the observed differences in Tr1 marker expression, anergy and cytokine production, the suppression capacity of T(stim-DC^{IL-10}) was comparable to that of T(unstim-DC^{IL-10}) (FIG. 37). Overall, we concluded that Tr1 cells induced by activated DC^{IL-10} are as powerful as the ones induced by unstimulated DC^{IL-10}, and thus TLR stimulation does not alter DC^{IL-10} tolerogenic potentials.

The interaction between CD47 on LV particles with its ligand Sirp-α on target cells impaired transduction efficiency by the reduction of LV particles uptake via phagocytosis. Thus, the inventors verified if the expression of CD47 on LV particles modified the efficiency of transduction of human DC. To this end, they performed transduction of DC precursors with LV particles harboring different levels of human-CD47 (huCD47) on the surface (huCD47-High LV>LV>HuCD47-free LV). Interestingly, LV-mediated transduction of Sirp-a expressing DC precursors was significantly increased using huCD47-free LV particles (FIG. 39).

### REFERENCES

1. Steinman L. (2010) J Intern Med. 267: 441-51. doi: 10.1111/j.1365-2796.2010.02224.x
2. Sakaguchi S. (1995) I J Immunol. 155: 1151-64.
3. Groux H. (1997) Nature. 389: 737-42.
4. Gregori S (2011) Tissue Antigens. 77: 89-99. doi: 10.1111/j.1399-0039.2010.01615.x
5. Amodio G, (2012) Frontiers in Immunology. 3. doi: 10.3389/fimmu.2012.00233
6. Osorio F, (2015) Front Immunol. 6: 535. doi: 10.3389/fimmu.2015.00535
7. Raker V K, (2015) Front Immunol. 6: 569. doi: 10.3389/fimmu.2015.00569
8. Liu J, Cao X (2015) J Autoimmun. 63: 1-12. doi: 10.1016/j.jaut.2015.07.011
9. Moreau A, (2012) Front Immunol. 3: 218. doi: 10.3389/fimmu.2012.00218
10. Giannoukakis N, (2011) Diabetes Care. 34: 2026-32. doi: 10.2337/dc11-0472
11. Bell G M, (2017) Ann Rheum Dis. 76: 227-34. doi: 10.1136/annrheumdis-2015-208456
12. Ten Brinke A, (2015) Mediators Inflamm. 2015: 471719. doi: 10.1155/2015/471719
13. Moreau A, (2012) EurJ Immunol. 42: 2881-8. doi: 10.1002/eji.201142325
14. Stoop J N, (2010) Arthritis Rheum. 62: 3656-65. doi: 10.1002/art.27756
15. Thomas D C, (2013) Diabetes. 62: 3132-42. doi: 10.2337/db12-1740
16. Schroers R, (2000) Mol Ther. 1: 171-9. doi: 10.1006/mthe.2000.0027
17. Breckpot K (2003) J Gene Med. 5: 654-67. doi: 10.1002/jgm.400
18. Tan P H, (2005) Blood. 105: 3824-32. doi: 10.1182/blood-2004-10-3880
19. Rowe H M, (2006) Mol Ther. 13: 310-9. doi: 10.1016/j.ymthe.2005.08.025
20. Boks M A, (2012) Clin Immunol. 142: 332-42. doi: 10.1016/j.clim.2011.11.011
21. Naranjo-Gomez M, (2011) J Transl Med. 9: 89. doi: 10.1186/1479-5876-9-89
22. Gregori S, (2010) Blood. 116: 935-44. doi: 10.1182/blood-2009-07-234872
23. Amodio G, (2015) Haematologica. 100: 548-57. doi: 10.3324/haematol.2014.113803
24. Amodio G, (2012) Transplantation research. 1: 14. doi: 10.1186/2047-1440-1-14
25. Pacciani V, (2010) J Allergy Clin Immunol. 125: 727-36. doi: S0091-6749(09)01796-5
26. Besche V, (2010) J Gene Med. 12: 231-43. doi: 10.1002/jgm.1436
27. Pallotta M T, (2014) J Cell Mol Med. 18: 2082-91. doi: 10.1111/jcmm.12360
28. Geissler E K, (2013) Curr Opin Organ Transplant. 18: 408-15. doi: 10.1097/MOT.0b013e328363319d
29. Tang Q, (2017) J Clin Invest. 127: 2505-12. doi: 10.1172/JCI90598
30. Safinia N, (2018) Front Immunol. 9: 354. doi: 10.3389/fimmu.2018.00354
31. Riquelme P, (2018) Nat Commun. 9: 2858. doi: 10.1038/s41467-018-05167-8
32. Steinman R M, (2003) Annual review of immunology. 21: 685-711. doi: 10.1146/annurev.immunol.21.120601.141040
33. Bartel D P (2004) Cell. 116: 281-97.
34. Smyth L A, (2015) Immunology. 144: 197-205. doi: 10.1111/imm.12390
35. Lu C, (2011) Blood. 117: 4293-303. doi: 10.1182/blood-2010-12-322503
36. Ceppi M, (2009) Proc Natl Acad Sci USA. 106: 2735-40. doi: 10.1073/pnas.0811073106
37. Holmstrom K, (2010) Hum Immunol. 71: 67-73. doi: 10.1016/j.humimm.2009.10.001
38. Jin P, (2010) J Transl Med. 8: 4. doi: 10.1186/1479-5876-8-4
39. Amendola M, (2005) Nat Biotechnol. 23: 108-16.
40. Andolfi G, (2012) Molecular Therapy. 20: 1778-90. doi: 10.1038/mt.2012.71
41. Brown B D, (2007) Nat Biotechnol. 25: 1457-67. doi: 10.1038/nbt1372
42. Annoni A, (2009) Blood. 114: 5152-61. doi: 10.1182/blood-2009-04-214569
43. Diebold S S, (2001) Gene Ther. 8: 487-93. doi: 10.1038/sj.gt.3301433
44. Singer P A, (1984) EMBO J. 3: 873-7.
45. Cresswell P (1992) Curr Opin Immunol. 4: 87-92.
46. Yamamoto K, (1985) Immunogenetics. 21: 83-90.
47. Bakke 0, (1990) Cell. 63: 707-16.
48. Lotteau V, (1990) INature. 348: 600-5. doi: 10.1038/348600a0
49. Strubin M, (1986) EMBO J. 5: 3483-8.
50. Wolf P R, (1995) AnnuRev Cell Dev Biol. 11: 267-306. doi: 10.1146/annurev.cb.11.110195.001411
51. Pieters J (2000) Adv Immunol. 75: 159-208.
52. Zhong G, (1997) J Exp Med. 185: 429-38. doi: 10.1084/jem.185.3.429
53. Serr I, (2016) Nat Commun. 7: 10991. doi: 10.1038/ncomms10991
54. Delong T, (2016) Science. 351: 711-4. doi: 10.1126/science.aad2791
55. Thrower S L, (2009) Clin Exp Immunol. 155: 156-65. doi: 10.1111/j.1365-2249.2008.03814.x
56. van Lummel M, (2016) Molecule. Diabetes. 65: 732-41. doi: 10.2337/db15-1031
57. Martinuzzi E, (2008) Diabetes. 57: 1312-20. doi: 10.2337/db07-1594
58. Gottlieb P A, (2014) J Autoimmun. 50: 38-41. doi: 10.1016/j.jaut.2013.10.003
59. Yang J, (2006) J Immunol. 176: 2781-9. doi: 10.4049/jimmunol.176.5.2781
60. Scotto M, (2012) Diabetologia. 55: 2026-31. doi: 10.1007/s00125-012-2543-z
61. Dang M, (2011) J Immunol. 186: 6056-63. doi: 10.4049/jimmunol.1003815
62. Abulafia-Lapid R, (1999) J Autoimmun. 12: 121-9. doi: 10.1006/jaut.1998.0262
63. Geluk A, (1998) J Autoimmun. 11: 353-61. doi: 10.1006/jaut.1998.0207
64. Law S C, (2012) Arthritis Res Ther. 14: R118. doi: 10.1186/ar3848
65. Andersson E C, (1998) Proc Natl Acad Sci USA. 95: 7574-9. doi: 10.1073/pnas.95.13.7574
66. Backlund J, (2002) Proc Natl Acad Sci USA. 99: 9960-5. doi: 10.1073/pnas.132254199
67. Lutterotti A, (2013) STM e. 5: 188ra75. doi: 10.1126/scitranslmed.3006168
68. Wucherpfennig K W, (1997) J Clin Invest. 100: 1114-22. doi: 10.1172/JCI119622
69. Camarca A, (2009) J Immunol. 182: 4158-66. doi: 10.4049/jimmunol.0803181
70. Tollefsen S, (2006) J Clin Invest. 116: 2226-36. doi: 10.1172/JC127620
71. Christophersen A, (2016) J Immunol. 196: 2819-26. doi: 10.4049/jimmunol.1501152
72. Christophersen A, (2019) Nat Med. 25: 734-7. doi: 10.1038/s41591-019-0403-9
73. Arai T, (2001) Blood. 98: 1889-96. doi: 10.1182/blood.v98.6.1889
74. Ito H, (2000) Hum Immunol. 61: 366-77.
75. deMoerloose P (2017) Haematologica. 102: 1324-32. doi:10.3324/haematol.2017.170381
76. Veldman C M, (2004) J Immunol. 172: 3883-92. doi: 10.4049/jimmunol.172.6.3883
77. Mouquet H, (2006) A J Immunol. 177: 6517-26. doi: 10.4049/jimmunol.177.9.6517
78. Mattapallil M J, (2011) J Immunol. 187: 1977-85. doi: 10.4049/jimmunol.1101247
79. Rai G, (2001) Exp Mol Pathol. 70: 140-5. doi: 10.1006/exmp.2000.2338
80. Sharma S, (2017) Front Immunol. 8: 1370. doi: 10.3389/fimmu.2017.01370
81. Vaughan K, (2012) Autoimmune Dis. 2012: 403915. doi: 10.1155/2012/403915
82. Ooi J D (2012) Proc Natl Acad Sci USA. 109: E2615-24. doi: 10.1073/pnas.1210147109
83. Yang J. (2008) Kidney international. 74: 1159-69. doi: 10.1038/ki.2008.309
84. Follenzi A., (2000) Nat Genet. 25: 217-22. doi: 10.1038/76095
85. Locafaro G. (2017) Mol Ther. 25: 2254-69. doi: 10.1016/j.ymthe.2017.06.029
86. Berger G. (2011) Nat Protoc. 6: 806-16. doi: 10.1038/nprot.2011.327
87. Amabile A., (2016) Cell. 167: 219-32 e14. doi: 10.1016/j.cell.2016.09.006
88. Lombardo A. (2011) Nat Methods. 8: 861-9. doi: 10.1038/nmeth.1674
89. Montini E. (2009) T J Clin Invest. 119: 964-75. doi: 10.1172/JC137630
90. Gagliani N. (2013) Nature medicine. doi: 10.1038/nm.3179
91. Santoni de Sio F R. (2018) JACI 142: 1909-21 e9. doi: 10.1016/j.jaci.2018.03.015
92. Comi M. (2019) Cell Mol Immunol. doi: 10.1038/s41423-019-0218-0
93. Sato S. J Immunol. 1997; 159:3278-3287

1. A genetically modified dendritic cell or a precursor cell thereof modified with a nucleic acid construct said construct comprising:
a nucleic acid sequence a) encoding a chimeric protein consisting of a human invariant chain fused to an antigenic peptide or protein or an antigenic fragment thereof, said sequence a) being operatively linked to a first promoter and optionally to a first transcription regulatory sequence and
a nucleic acid sequence b) encoding an immuno-modulatory protein, said sequence b) being optionally operatively linked to a second promoter and optionally operatively linked to a second transcription regulatory sequence.

2. The genetically modified dendritic cell or precursor cell thereof according to claim 1 wherein the sequence a) further comprises at its 3′ end an miRNA target sequence.

3. The genetically modified dendritic cell or precursor cell thereof according to claim 1 wherein said the first promoter and the second promoter are the same or different.

4. The genetically modified dendritic cell or precursor cell thereof according to claim 1 wherein said nucleic acid construct further comprises a sequence encoding a marker, which is optionally a selectable marker.

5. The genetically modified dendritic cell or precursor cell thereof according to claim 1 wherein the human invariant chain is Iip33, Iip41, Iip35 or Iip43.

6. The genetically modified dendritic cell or precursor cell thereof according to claim 1 wherein said antigenic peptide or protein or antigenic fragment thereof is derived from an auto-antigen and/or a non-harmful antigen and/or an allergen.

7. The genetically modified dendritic cell or precursor cell thereof according to claim 1 wherein said antigenic peptide or protein or antigenic fragment thereof is selected from the group of immunodominant peptides as described in Table 2.

8. The genetically modified dendritic cell or precursor cell thereof according to claim 1 wherein said immuno-modulatory protein is selected from the group consisting of: IL-10, indoleamine 2,3-dioxygenase (IDO), PDL-1, PDL-2, ILT-3, ILT-4, HO-1, ICOS-L Gal9, HVME, HLA-G, HLA-E, IL-35, TGF-b, CTLA-4Ig, PGE2, TNFRs, Arg1, and mixtures thereof.

9. The genetically modified dendritic cell or precursor cell thereof according to claim 2 wherein the a miRNA target sequence is selected from the group targeting: miR-15a, miR-16-1, miR-17, miR-18a, miR-19a, miR-20a, miR-19b-1, miR-21, miR-29a, miR-29b, miR-29c, miR-30b, miR-31, miR-34a, miR-92a-1,miR-106a, miR-125a, miR-125b, miR-126, miR-142-3p, miR-146a, miR-150, miR-155, miR-181a, miR-223 and miR-424, and mixtures thereof, wherein said miRNA target sequence is optionally repeated.

10. The genetically modified dendritic cell or a precursor cell thereof according to claim 1 wherein said cell displays at least one of the following properties: modulates CD4+ and CD8+ T cell responses; modulates antigen-specific CD4+ and CD8+ T cell proliferation in vitro and/or in vivo; favors the generation of regulatory DC; favors the expansion of antigen-specific Tr1 and/or FOXP3+ Treg cells, is tolerogenic, presents antigen in the context of both MHC class I and class II.

11. The genetically modified dendritic cell or precursor cell thereof according to claim 1 wherein said nucleic acid construct is inserted into a vector, optionally a lentiviral vector or a mono- or bi-directional vector.

12. (canceled)

13. A method for the prevention and/or treatment of a condition selected from the group consisting of: graft versus host disease, organ rejection, autoimmune disease, allergic disease, inflammatory or auto-inflammatory disease, immune response induced by gene therapy, prevention of immune responses against protein replacement therapy, or lysosomal storage disorders or hemophilia, comprising administering the genetically modified dendritic cell or precursor cell thereof of claim 1 to a patient in need thereof.

14. The method according to claim 13 wherein the autoimmune disease is selected from the group consisting of: type 1 diabetes mellitus, autoimmune enteropathy, rheumatoid arthritis, systemic lupus erythematosus, multiple sclerosis, autoimmune myositis, psoriasis, Addison's disease, Grave's disease, Sjogren's syndrome, Hashimoto's thyroiditis, myasthenia gravis, vasculitis, pernicious anemia, celiac disease, autoimmune hepatitis, alopecia areata, pemphigus vulgaris, vitiligo, aplastic anemia, autoimmune uveitis, Alopecia Areata, Amyotrophic Lateral Sclerosis (Lou Gehrig's), Ankylosing Spondylitis, Anti-GBM Nephritis, Antiphospholipid Syndrome, Osteoarthritis, Autoimmune Active Chronic Hepatitis, Autoimmune Inner Ear Disease (AIED), Balo Disease, Behcet's Disease, Berger's Disease, Bullous Pemphigoid, Cardiomyopathy, Chronic Fatigue Immune Dysfunction Syndrome, Churg Strauss Syndrome, Cicatricial Pemphigoid, Cold Agglutinin Disease, Colitis Cranial Arteritis, Crest Syndrome, Crohn's Disease, Dego's Disease, Dermatomyositis & JDM, Devic Disease, Eczema, Essential Mixed Cryoglobulinemia, Eoscinophilic Fascitis, Fibromyalgia—Fibromyositis, Fibrosing Alveolitis, Giant Cell Arteritis, Glomerulonephritis, Goodpasture's Disease, Guillain-Barre Syndrome, Hashimoto's Thyroiditis, Hepatitis, Hughes Syndrome, Idiopathic Pulmonary Fibrosis, Idiopathic Thrombocytopenic Purpura, Irritable Bowel Syndrome, Kawasaki's Disease, Lichen Planus, Lupoid Hepatitis, Lupus/SLE, Lyme Disease, Meniere's Disease, Mixed Connective Tissue Disease, Myositis: Juvenile Myositis (JM), Juvenile dermatomyositis (JDM), and Juvenile Polymyositis (JPM), Osteoporosis, Pars Planitis, Pemphigus Vulgaris, Polyglandular Autoimmune Syndromes, Polymyalgia Rheumatica, Polymyositis, Primary Biliary Cirrhosis, Primary Sclerosis Cholangitis, Psoriasis, Raynaud's Syndrome, Reiter's Syndrome, Rheumatic Fever, Rheumatoid Arthritis, Scleritis, Scleroderma, Sticky Blood Syndrome, Still's Disease, Stiff Man Syndrome, Sydenham's Chorea, Takayasus Arteritis, Temporal Arteritis, Ulcerative Colitis, Uveitis, Vasculitis, Wegener's Granulomatosis and Wilson's Syndrome, preferably the autoimmune disease is vasculitis such as catastrophic anti-phospholipid syndrome (also named Asherson's syndrome), Giant Cell Arteritis and anti-ANCA vasculitis or myasthemia gravis, refractory celiac disease, autoimmune uveitis such as Behcet's Disease, pemphigus vulgaris, giant cell myocarditis, Graves' disease, Addison's disease and granulomatosis with polyangiitis.

15. The method according to claim 13 wherein the allergic disease is asthma, atopic allergy or atopic dermatitis.

16. The method according to claim 13 wherein the inflammatory or autoinflammatory disease is a chronic inflammatory disease, optionally the chronic inflammatory disease is selected from the group consisting of: inflammatory bowel disease, Chron's disease, ulcerative colitis, celiac disease.

17. (canceled)

18. A nucleic acid construct comprising:
a nucleic acid sequence a) encoding a chimeric protein consisting of a human invariant chain fused to an antigenic peptide or protein or an antigenic fragment thereof, said sequence a) being operatively linked to a first promoter and optionally to a first transcription regulatory sequence and
a nucleic acid sequence b) encoding an immuno-modulatory protein, said sequence b) being optionally operatively linked to a second promoter and optionally operatively linked to a second transcription regulatory sequence.

19. A vector comprising the nucleic acid construct as defined in claim 18, optionally said vector is a lentiviral vector or a mono- or bi-directional vector, optionally the vector is produced using an enveloped viral particle expressing Vpx and/or the vector is produced using a packaging cell wherein said packaging cell is genetically engineered to decrease expression of CD47.

20. An in vitro method to produce the genetically modified dendritic cell or a precursor cell thereof claim 1 comprising the steps of:
a. Isolating PBMCs from a subject;
b. Isolating CD14⁺ cells from said isolated PBMCs;
c. Incubating said isolated CD14+ cells with an effective amount of Vpx;
d. Transducing said isolated CD14⁺ cells with the vector according to claim 19.

21. The in vitro method according to claim 20 wherein step d. is performed in the presence of an effective amount of an agent, wherein optionally the agent is IL-4 or Granulocyte-macrophage colony-stimulating factor (GM-CSF) or IL-10, and the amount of IL-4, of GM-CSF and of IL-10 is optionally between 1 and 1000 ng.

22. The in vitro method according to claim 20 wherein the PBMCs are isolated from peripheral blood or from leukapheresis.

23. The in vitro method according to claim 20 wherein the vector is a lentiviral vector, and optionally the amount of said lentiviral vector is between 1 to 100 MOI.

24. A genetically modified dendritic cell or a precursor cell thereof obtainable by the method of claim 20.

25. An in vitro method to produce IL-10-producing CD49b⁺ LAG-3⁺ Tr1 cells comprising the steps of:
a) isolating PBMCs from a blood sample of a subject;
b) exposing said isolated PBMCs in appropriate culture conditions with an effective amount of a genetically modified dendritic cell or a precursor cell thereof as defined in claim 1.

26. The in vitro method according to claim 25 wherein the ratio PBMC:genetically modified dendritic cell or precursor thereof is between 5:1 and 10:1.

27. An IL-10-producing CD49b⁺ LAG-3⁺ Tr1 cell obtainable by the method of claim 25, optionally for medical use.

28. An in vitro method to produce antigen-specific FOXP3⁺ T cells comprising the steps of:
a) isolating PBMCs from a blood sample of a subject;
b) exposing said isolated PBMCs in appropriate culture conditions with an effective amount of a genetically modified dendritic cell or precursor cell thereof as defined in claim 1.

29. The in vitro method according to claim 28 wherein the genetically modified cell expresses at least indoleamine 2,3-dioxygenase (IDO).

30. The antigen-specific FOXP3+ T cell obtainable according to the method of claim 28, optionally for medical use.

31. A pharmaceutical composition comprising the genetically modified cell as defined in claim 1 and a pharmaceutically acceptable carrier.

32. The pharmaceutical composition according to claim 31 further comprising a therapeutic agent.

33. A genetically modified dendritic cell or a precursor cell thereof modified with a nucleic acid construct said construct comprising a nucleic acid sequence encoding IL-10, said sequence being operatively linked to a promoter and optionally to a transcription regulatory sequence and/or optionally to a marker, optionally a selectable marker.

34. A genetically modified dendritic cell or a precursor cell thereof modified with a nucleic acid construct said construct comprising:
a nucleic acid sequence a) encoding a chimeric protein consisting of a human invariant chain fused to an antigenic peptide or protein or an antigenic fragment thereof, said sequence a) being operatively linked to a first promoter and optionally to a first transcription regulatory sequence and
a nucleic acid sequence encoding at least one miRNA target sequence.

35. The genetically modified dendritic cell or a precursor cell thereof according to claim 33 for use in organ and/or bone marrow transplant and/or for the prevention and/or treatment of graft rejection and/or graft versus host disease.

36. The genetically modified dendritic cell or a precursor cell thereof according to claim 34 for use in the prevention and/or treatment of a condition selected from the group consisting of: autoimmune disease, allergic disease, inflammatory disease, immune response induced by gene therapy.
